(19) United States
(12) Patent Application Publication
(10) Pub. No.: US 2014/0093457 A1
(43) Pub. Date: Apr. 3, 2014

(54) Compositions, methods and kits for remineralization and inhibition of dental caries in teeth

(71) Applicant: President and Fellows of Harvard College, Cambridge, MA (US)

(72) Inventor: Shigemi Nagai, Lexington, MA (US)

(73) Assignee: PRESIDENT AND FELLOWS OF HARVARD COLLEGE, Cambridge, MA (US)

(21) Appl. No.: 13/832,445

(22) Filed: Mar. 15, 2013

Related U.S. Application Data
(63) Continuation of application No. PCT/US2011/064150, filed on Dec. 9, 2011.
(60) Provisional application No. 61/421,477, filed on Dec. 9, 2010.

(51) Int. Cl. A61K 6/00; A61K 33/42

(52) U.S. Cl. CPC A61K 6/0058; A61K 33/42
USPC 424/9.6; 435/375; 424/601; 562/13; 423/304

(57) ABSTRACT

Methods, compositions and kits are provided for enhancing remineralization of a tooth or bone containing hydroxyapatite and inhibiting caries progression or loss of hydroxyapatite using a bisphosphonate or pyrophosphonate. The tooth or tooth surface contains a trauma or defect, for example the tooth contains a caries that is identified using a detectable probe. The bisphosphonate or pyrophosphonate is contacted to the tooth and/or oral cavity and binds to the hydroxyapatite material in the tooth or bone and prevents loss of hydroxyapatite material or strengthens the hydroxyapatite. The amount and extent of enhanced remineralization or of inhibition of caries progression and loss of hydroxyapatite are determined by techniques including photography, light microscopy and fluorescence microscopy.

### RELATED APPLICATION

This utility application claims the benefit of international application number PCT/US2011/064150 filed Dec. 9, 2011 entitled, “Compositions, methods and kits for remineralization and inhibition of dental caries in teeth” by Shigemi Nagai, which claims the benefit of U.S. provisional application Ser. No. 61/421,477 inventor Shigemi Nagai filed Dec. 9, 2010 in the United States Patent and Trademark Office entitled, “Compositions, methods and kits for enhancing remineralization and inhibiting caries progression and breakdown of hydroxyapatite in teeth”, each of which is hereby incorporated by reference in its entirety.

### TECHNICAL FIELD

Compositions, method and kits and are provided for use of bisphosphonate compositions or pyrophosphonate compositions that bind preferentially to hydroxyapatite in teeth to enhance remineralization, to inhibit caries progression, and to inhibit loss of hydroxyapatite.

### BACKGROUND

Caries and periodontal disease remain the main reasons for tooth loss worldwide, despite positive effects of preventive measures to reduce caries primarily through the application of fluoride. If detected early before demineralization of the tooth surface has reached the dentin, an incipient caries lesion can be cured by remineralization.

If however the lesion has progressed into the dentin, restorative procedures such as placing amalgam or composite fillings into the treated caries become necessary. Such restorative procedures are in general more invasive and represent a much greater expense to a patient or a third party provider than preventive or curative procedures. Thus, early detection of carious lesions is a key element in economic and efficient prevention and treatment of dental caries.

Indices and methods of conducting surveys for the level of dental disease were developed between 1900 and 1950. Modern epidemiological studies began after that, and many reliable studies have been conducted since 1960. Concluding remarks of the Symposium of the ORCA Caries Diagnosis Working Group state that the development of methods for determining whether a carious lesion is stable or progressing is a priority in caries research.

The poor diagnostic performance of conventional caries detection methods has prompted the research community to develop quantitative detection methods, such as electrical conductance measurements, light scattering methods, and laser fluorescence methods, in addition to the X-ray technique which is the current standard. Motivations for this development include that quantitative methods detect lesions at an earlier stage than conventional methods, are more reliable than qualitative measurements, and provide methods for monitoring the course of disease in a way that is non-detrimental to the patient.

A systematic review of diagnostic methods prepared for the 2001 National Institutes of Health Consensus Development Conference on ‘Diagnosis and Management of Dental Caries through Life’ was unable to establish relative efficacies of various methods currently used to detect dental caries.

New methods and criteria are needed for diagnosis and prognosis of caries and periodontal disease, and also the remineralization of teeth. Improved methods of inhibiting caries progression and hydroxyapatite breakdown in teeth offer the opportunity for improved health and also tooth appearance.

### SUMMARY

An embodiment of the invention provides a method for enhancing remineralization of a tooth of a subject and inhibiting caries progression and loss of hydroxyapatite, the method including: contacting the tooth or oral cavity of the subject with a composition including at least one selected from bisphosphonate and a pyrophosphonate, such that the composition binds to the tooth, an area of the tooth having a caries lesion or both; such that the tooth of the subject includes less porous hydroxyapatite, and the caries progression or the loss of the hydroxyapatite is inhibited, compared to a tooth not contacted. The subject is a mammal, for example a human. In related embodiments of the method, the compound binds to the tooth or tooth surface and reduces the porosity of the hydroxyapatite. In various embodiments, the composition is identified or visualized by a dye, stain or indictor that provides a fluorescent emission signal that is separate and outside the autofluorescence wavelengths of the tooth comprising enamel or dentin. For example, the caries lesion is an interproximal caries.

In a related embodiment of the method, contacting involves the composition binding to the hydroxyapatite on the tooth, for example hydroxyapatite that is exposed on a decayed tooth. In a related embodiment, the tooth includes a tooth surface for example an occlusal surface or buccal surface, or the tooth comprises a tooth component for example enamel or dentin.

In a related embodiment of the method, the composition binds to hydroxyapatite in other bone types or areas in the oral cavity or body, for example the areas of bone include mandibular bone or maxillary bone.

In a related embodiment of the method, the tooth includes a defect associated with a trauma or a disease, for example the defect includes dental caries, a decayed tooth, a chipped tooth, or a tooth erosion. For example, the defect is an early stage dental caries in the enamel that is detected using a fluorescent probe that binds to the caries.

In related embodiments of the method, the composition includes at least one molecule or moiety selected from: a dye, a stain, a pigment, a polymer, a nanoparticle, and a peptide. For example, the dye includes a cyanine dye.

In an embodiment of the method, the composition is attached to the molecule by an ionic bond. Alternatively, the composition is attached to the molecule by a covalent bond.

In related embodiments of the method, the molecule includes a fluorescent composition. For example, the molecule includes at least one selected from: a tetracycline, HiLyte Fluor, a Qdot, Cy7, CardioGreen (ICG), IR820 (ICG), Far-Green two, AngioSense 750, Genhance 750, AngioSpark, 750, Alexa. Fluor 750, Indocyanine Green, Doxorubicin, Riboflavin, Chlorophyll A, a bacterial Chlorophyll, and a porphyrin. Alternatively, the molecule includes a bioluminescent composition or a composition that absorbs light for example a colloidal gold.

In a related embodiment of the method, the composition includes at least one selected from: OsteoSense 750, zoledronate, aledronate, pamidronate, risedronate, ibandronate, incadronate, minodronate, olpadronate, neridronate, etidoronate, clodronate, tiludronate, methylene bisphosphonate, and a derivative thereof for example a salt or an ester.

In a related embodiment of the method, the composition further includes at least one component and/or substance selected from the group of: an organic acid, a carbonate, an alcohol, a glycol, a glycerol, a sugar, an inorganic acid, a salt, an ester, a hydrate, a solvate, a polymer, and a derivative thereof. In a related embodiment of the method, the composition further includes at least one agent, for example the agent includes at least one from the group of: an antibacterial agent, an anti-microbial agent, and an anti-fungal agent. For example, the composition further includes a fluoride composition. In a related embodiment, the composition includes an antiseptic and/or an agent that inhibits plaque formation, for example the composition includes chlorhexidine gluconate.

In a related embodiment of the method, the composition is formulated for delivery to the tooth or the oral cavity for example the composition is formulated at least one from the group of: a gel, a solution, an emulsion, an ointment, a paste, a cream, a gel, a powder, an aerosol, a patch, a strip, and a tape. For example, the composition is formulated as a dental patch that locally delivers the composition when attached to the tooth or to the oral cavity.

In a related embodiment, the method further includes after contacting the tooth or oral cavity with the compound: illuminating the tooth, the oral cavity or both; and detecting a wavelength of fluorescence emission, thereby monitoring the amount or extent of the inhibition of caries progression or the loss of hydroxyapatite of the tooth. In a related embodiment, the method further includes after detecting the wavelength, contacting the tooth with additional amounts of the composition or contacting with a remineralization agent. Alternatively, detecting includes detecting absorbance or bioluminescence.

In a related embodiment of the method, contacting is performed in vivo. Alternatively, contacting is performed ex vivo.

In a related embodiment, the method further includes prior to contacting the tooth or the oral cavity with the compound, visualizing a defect in the tooth. For example, the method includes visualizing an area of demineralized enamel.

In a related embodiment of the method, visualizing includes at least one technique selected from: radiography, microscopy, and photography. For example, the method includes obtaining a bite wing radiological image.

In a related embodiment, the method further includes prior to contacting the tooth or oral cavity with the composition at least one step selected from: removing loose or diseased bone material, cleaning the tooth, comparing the bone material to a previous image of the tooth, detecting a caries in the tooth, and rinsing the tooth with a fluid.

In an embodiment of the method, contacting includes at least one technique selected from the group of: spraying, injecting, immersing, and adding dropwise. In an embodiment of the method, contacting includes topically applying the composition with a dental hand-held device such as a pick, a hook, or a spatula.

In a related embodiment of the method, contacting includes adding the composition using an applicator, for example the applicator is selected from: a syringe, a brush, a swab, a gauze, a sprayer, a sponge, a bite wing plate, a dropper, a gel, a strip, a tape, a tray, and a string.

In a related embodiment, the method further includes after contacting the tooth or the oral cavity of the subject with the compound, contacting the tooth or oral cavity with a remineralization agent, for example the remineralization agent including at least one of calcium or phosphate.

An embodiment of the invention provides a composition for enhancing remineralization of a tooth of a subject and inhibiting caries progression and loss of hydroxyapatite, the composition including: at least one of a bisphosphonate and a pyrophosphonate, such that the composition binds to hydroxyapatite in the tooth and an area of the tooth having a caries lesion. In certain embodiments, the composition involves a dilution from a stock solution of about 1:2 to about 1:10, about 1:10 to about 1:200, or about 1:200 to about 1:400. For example, the stock solution is a one milligram per milliliter (mg/mL) stock solution. Alternatively, the bisphosphonate or pyrophosphonate includes an amount between about 10 percent and 25 percent by weight, about 25 percent and 50 percent by weight, about 50 percent and 80 percent by weight, or about 80 percent and 99 percent by weight.

In a related embodiment, the composition includes an ionic bond to a molecule selected from: a dye, a stain, a pigment, a polymer, a nanoparticle, and a peptide. Alternatively, the compound includes a covalent bond to the molecule.

In a related embodiment, the compound is formulated as a gel, a foam, a paste, a fluid, an emulsion, a patch, a capsule, an ointment, or a pill. In related embodiments, the compound is formulated for slow release over a period of time. For example, the compound is formulated to be released over a period of seconds, minutes, or hours.

In a related embodiment, the compound includes at least one selected from the group of: OsteoSense 750, zoledronate, aledronate, pamidronate, risedronate, ibandronate, incadronate, minodronate, olpadronate, neridronate, etidoronate, clodronate, tiludronate, and a derivative thereof for example a salt or an ester.

In a related embodiment, the composition is formulated for topical administration to the tooth or oral cavity. In a related embodiment, the compound is formulated to be nom toxic, bioresorbable, or biocompatible to the subject.

In a related embodiment, the composition is formulated to remain in an oral cavity for at least five minutes, at least 15 minutes, at least 30 minutes, at least one hour, at least two hours, at least four hours, at least six hours. For example, the composition is formulated to remain in the oral cavity in the presence of saliva or fluids consumed by the subject.

In a related embodiment, the compound is formulated with at least one pharmaceutically acceptable buffer, salt, preservative, solvent, emulsifier, diluent, oil, polymer, binding agent, or filler.

In an embodiment of the invention, the composition is effective for remineralizing or inhibiting caries progression for lesions having a depth of 20-500 micrometers. In various embodiments, the depth of the lesion is at least about: 20 micrometers, 50 micrometers, 100 micrometers, 150 micrometers, 200 micrometers, 300 micrometers, 350 micrometers, 400 micrometers, and 500 micrometers.

An embodiment of the invention provides a kit for enhancing remineralization of a tooth of a subject and inhibiting caries progression and loss of hydroxyapatite, the kit including: a composition including at least one of a bisphosphonate and a pyrophosphonate; and, a container. In an embodiment, the kit includes any of the compositions described herein.

In a related embodiment, the kit further includes an applicator for the composition. For example the applicator is selected from: a syringe, a brush, a swab, a sprayer, a sponge, a bite wing plate, a dropper, a strip, a tape, a tray, and a string. In a related embodiment, the kit further includes instructions for use.

In a related embodiment of the kit, the composition includes at least one from the group of: OsteoSense 750, aledronate, pamidronate, resedronate, ibandronate, etidoronate, clodronate, tiludronate, methylene bisphosphonate, and a derivative thereof for example a salt or an ester.

In a related embodiment, the kit further induces a fluid for removing excess composition from the tooth. In a related embodiment of the kit, the composition is formulated as a gel, a paste, a fluid, an emulsion, a patch, a capsule, an ointment, or a pill.

An embodiment of the invention provides a product containing a composition for enhancing remineralization of a tooth of a subject and inhibiting caries progression and loss of hydroxyapatite from the tooth, the composition comprising: at least one of a bisphosphonate and a pyrophosphonate, wherein the composition binds to the hydroxyapatite in the tooth and an area of the tooth having a caries lesion. In various embodiments, the product includes any of the compositions described herein including bisphosphonate compounds and/or pyrophosphonate compounds.

An embodiment of the invention provides use of any of the composition described herein in preparation of a medicament for enhancing remineralization of a tooth of a subject and inhibiting caries progression and loss of hydroxyapatite from the tooth.

An embodiment of the invention provides a method of making a composition for enhancing remineralization of a tooth of a subject and inhibiting caries progression and loss of hydroxyapatite from the tooth, the composition comprising: at least one of a bisphosphonate and a pyrophosphonate, such that the composition binds to hydroxyapatite in the tooth and an area of the tooth having a caries lesion. In various embodiments, the method involves making any of the compositions described herein, for example by covalent bonding or ionic bonding of the bisphosphonate and/or the pyrophosphonate to a molecule such as a dye, a stain, or an indicator. In various embodiments of the method, the composition optionally further comprises the molecule comprising at least one selected from the group consisting of: a tetracycline, HiLyte Fluor, a Qdot, Cy7, CardioGreen (ICG), IR820 (ICG), Far-Green two, AngioSense 750, Genhance 750, AngioSpark, 750, Alexa Fluor 750, Indocyanine Green, Doxorubicin, Riboflavin, Chlorophyll A, a bacterial Chlorophyll, and a porphyrin. For example, the molecule is a dye or marker for visualizing the composition bound to the caries or tooth, or for visualizing the extent of remineralization and inhibition of caries progression. In various embodiments of the method, the bisphosphonate comprises

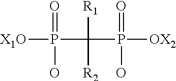

such that R₁ and R₂ are independently synthesized from a (C₁-C₁₈)alkyl, a (C₁-C₁₈)heteroalkyl, a (C₁-C₁₈)alkoxy, a (C₁-C₁₈)heteroalkyl, a (C₆-C₁₀)aryl, a (C₁-C₉)heteroaryl, and a (C₆-C₁₀)aryl(C₁-C₆)alkyl. For example R₁ and R₂ include functional groups including aliphatic groups, olefin groups (monounsaturated and polyunsaturated), and heteroatom groups (e.g., oxygen, nitrogen, and fluoride). In the above structural formula, X₁ and X₂ are independently synthesized from: (C₆-C₁₀)aryl, (C₁-C₉)heteroaryl, (C₆-C₁₀)aryl(C₁-C₆)alkyl, (C₁-C₉)heteroaryl(C₁-C₆)alkyl, (C₆-C₁₀)aryl(C₆-C₁₀)aryl, (C₁-C₉)heteroaryl(C₆-C₁₀)aryl, (C₆-C₁₀)aryl(C₁-C₉)heteroaryl, (C₁-C₉)heteroaryl(C₁-C₉)heteroaryl, (C₆-C₁₀)aryloxy(C₆-C₁₀)aryl, (C₁-C₉)heteroaryloxy(C₆-C₁₀)aryl, (C₆-C₁₀)aryloxy(C₁-C₉)heteroaryl, (C₁-C₉)heteroaryloxy(C₁-C₉)heteroaryl, (C₆-C₁₀)aryloxy(C₁-C₆)alkyl, (C₁-C₉)heteroaryloxy(C₁-C₆)alkyl, (C₆-C₁₀)aryl(C₁-C₆)alkyl(C₆-C₁₀)aryl, (C₁-C₉)heteroaryl(C₁-C₆)alkyl(C₆-C₁₀)aryl, (C₆-C₁₀)aryl(C₁-C₆)alkyl(C₁-C₉)heteroaryl, (C₁-C₉)heteroaryl(C₁-C₆)alkyl(C₁-C₉)heteroaryl, (C₆-C₁₀)aryl(C₁-C₆)alkoxy(C₆-C₁₀)aryl, (C₁-C₉)heteroaryl(C₁-C₆)alkoxy(C₆-C₁₀)aryl, (C₆-C₁₀)aryl(C₁-C₆)alkoxy(C₁-C₉)heteroaryl, (C₁-C₉)heteroaryl(C₁-C₆)alkoxy(C₁-C₉)heteroaryl, (C₆-C₁₀)aryloxy(C₁-C₆)alkyl(C₆-C₁₀)aryl, (C₁-C₉)heteroaryloxy(C₁-C₆)alkyl(C₆-C₁₀)aryl, (C₆-C₁₀)aryloxy(C₁-C₆)alkyl(C₁-C₉)heteroaryl, (C₁-C₉)heteroaryloxy(C₁-C₆)alkyl(C₁-C₉)heteroaryl, (C₆-C₁₀)aryl(C₆-C₁₀)aryl(C₁-C₆)alkyl, (C₁-C₉)heteroaryl(C₆-C₁₀)aryl(C₁-C₆)alkyl, (C₆-C₁₀)aryl(C₁-C₉)heteroaryl(C₁-C₆)alkyl, (C₁-C₉)heteroaryl(C₁-C₉)heteroaryl(C₁-C₆)alkyl, (C₆-C₁₀)aryl(C₁-C₆)alkoxy(C₁-C₆)alkyl, or (C₁-C₉)heteroaryl(C₁-C₆)alkoxy(C₁-C₆)alkyl, such that, independently, each of the ring carbon atoms of the (C₆-C₁₀)aryl and (C₁-C₉)heteroaryl moieties that is capable of forming an additional bond by a group such as fluoro, chloro, bromo, (C₁-C₆)alkyl, (C₁-C₆)alkoxy, and perfluoro(C₁-C₃)alkyl, and perfluoro(C₁-C₃)alkoxy.

In various embodiments, the pyrophosphonate comprises

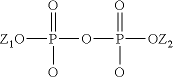

such that Z₁ and Z₂ are independently synthesized from: (C₆-C₁₀)aryl, (C₁-C₉)heteroaryl, (C₆-C₁₀)aryl(C₁-C₆)alkyl, (C₁-C₉)heteroaryl(C₁-C₆)alkyl, (C₆-C₁₀)aryl(C₆-C₁₀)aryl, (C₁-C₉)heteroaryl(C₆-C₁₀)aryl, (C₆-C₁₀)aryl(C₁-C₉)heteroaryl, (C₁-C₉)heteroaryl(C₁-C₉)heteroaryl, (C₆-C₁₀)aryloxy(C₆-C₁₀)aryl, (C₁-C₉)heteroaryloxy(C₆-C₁₀)aryl, (C₆-C₁₀)aryloxy(C₁-C₉)heteroaryl, (C₁-C₉)heteroaryloxy(C₁-C₉)heteroaryl, (C₆-C₁₀)aryloxy(C₁-C₆)alkyl, (C₁-C₉)heteroaryloxy(C₁-C₆)alkyl, (C₆-C₁₀)aryl(C₁-C₆)alkyl(C₆-C₁₀)aryl, (C₁-C₉)heteroaryl(C₁-C₆)alkyl(C₆-C₁₀)aryl, (C₆-C₁₀)aryl(C₁-C₆)alkyl(C₁-C₉)heteroaryl, (C₁-C₉)heteroaryl(C₁-C₆)alkyl(C₁-C₉)heteroaryl, (C₆-C₁₀)aryl(C₁-C₆)alkoxy(C₆-C₁₀)aryl, (C₁-C₉)heteroaryl(C₁-C₆)alkoxy(C₆-C₁₀)aryl, (C₆-C₁₀)aryl(C₁-C₆)alkoxy(C₁-C₉)heteroaryl, (C₁-C₉)heteroaryl(C₁-C₆)alkoxy(C₁-C₉)heteroaryl, (C₆-C₁₀)aryloxy(C₁-C₆)alkyl(C₆-C₁₀)aryl, (C₁-C₉)heteroaryloxy(C₁-C₆)alkyl(C₆-C₁₀)aryl, (C₆-C₁₀)aryloxy(C₁-C₆)alkyl(C₁-C₉)heteroaryl, (C₁-C₉)heteroaryloxy(C₁-C₆)alkyl(C₁-C₉)heteroaryl, (C₆-C₁₀)aryl(C₆-C₁₀)aryl(C₁-C₆)alkyl, (C₁-C₉)heteroaryl(C₆-C₁₀)aryl(C₁-C₆)alkyl, (C₆-C₁₀)aryl(C₁-C₉)heteroaryl(C₁-C₆)alkyl, (C₁-C₉)heteroaryl(C₁-C₉)heteroaryl(C₁-C₆)alkyl, (C₆-C₁₀)aryl(C₁-C₆)alkoxy(C₁-C₆)alkyl, or (C₁-C₉)heteroaryl(C₁-C₆)alkoxy(C₁-C₆)alkyl, such that, independently, each of the ring carbon atoms of the (C₆-C₁₀)aryl and (C₁-C₉)heteroaryl moieties that is capable of forming an additional bond by a group such as fluoro, chloro, bromo, (C₁-C₆)alkyl, (C₁-C₆)alkoxy, and perfluoro(C₁-C₃)alkyl, and perfluoro(C₁-C₃)alkoxy.

### BRIEF DESCRIPTION OF THE DRAWINGS

FIG. 1A and FIG. 1B are drawings showing the development/nature of a “white spot” (100) lesion, an early stage dental caries, that generally is formed by acid and bacteria invading the area between adjacent enamel prisms. An enamel prism (101) is a crystallized hydroxyapatite (HA) rod radiating from the underlying dentin. Enamel prism at a depth of about 100 micrometers or microns (μm) has a very strong and transparent layer. The white spot appears as a white area and shines through the enamel prism. A white spot forms within about 10 μm to about 100 μm, or within about 10 μm to about 150 μm of the surface of the enamel, or within one-half of the depth of enamel. The dotted line indicates one-half depth of the enamel prism. Early stage dental caries are not detected by traditional X-rays because X-ray detection requires that the lesion be larger than about one-half the depth of the enamel. A caries (102) that has extended beyond about one-half the enamel depth is visible and detected by X-rays.

FIG. 2 is a set of photographs showing an optical device system for detecting the probes that are bound to early stage caries lesions in teeth. The photographs show a light source (200) for illuminating a tooth to observe an early stage caries lesion bound to the detectable probe. A camera (201) captures the image of the tooth with an early stage caries lesion and bound probe. A computer (202) coordinates the emitting light source and camera, and further includes data analysis software (e.g., image detection algorithms, sample identification) and stores observed data. A stage (203) holds the sample, i.e., synthetic tooth, bovine tooth, or slice of enamel, having an early stage caries lesion for illumination. An optical fiber (204) transmits and focuses the electromagnetic light waves emitted by light source (200) and directs the waves at an area on the tooth that is on the stage (203).

FIG. 3 is a photograph showing absorption of light of a bismuth probe bound to an early stage caries lesion in a synthetic tooth preparation.

FIG. 4 is a photograph showing fluorescence of a Doxorubicin probe bound to an early stage caries lesion in a synthetic tooth preparation.

FIG. 5 is a photograph showing fluorescence of a Riboflavin probe bound to an early stage caries lesion in a synthetic tooth preparation.

FIG. 6 is a photograph showing fluorescence of a Chlorophyll A probe bound to an early stage caries lesion in a synthetic tooth preparation.

FIG. 7 is a photograph showing fluorescence of a Porphyrin probe bound to an early stage caries lesion in a synthetic tooth preparation.

FIG. 8 is a photograph showing bioluminescence of a luciferase and luciferin probe bound to an early stage caries lesion in a synthetic tooth preparation.

FIG. 9 is a set of photographs showing fluorescence of each of OsteoSense 750 (panels A, B, C and F), AngioSense 750 (panel D), Genhance 750 (panel E), and Chlorophyll A (panel G) probes bound to interproximal caries on teeth.

FIG. 10A and FIG. 10B are graphs showing quantification of fluorescence intensity (ordinate) of probes in a deep lesion (spot#1, left graphs) and a shallow lesion (spot #2, right graphs) and amount on time in minutes the fluorescence intensity was detected (abscissa). FIG. 10A and FIG. 10B show fluorescent intensity results for probes diluted 1:100 and 1:10, respectively. The probes contacted and bound to the lesions are CardioGreen (diamonds), IR820 (rectangles), Cy7 (triangles), and OsteoSense 750 (-x-).

FIG. 11A-FIG. 11D are photographs that shows fluorescence over a period of time (minutes, min) of probes (Cy7, OsteoSense 750 and IR(820)) bound to caries lesions on demineralized enamel surfaces.

FIG. 11A shows fluorescence from zero (0) minutes to 30 minutes for Cy7 diluted 1:10 and bound to caries on a demineralized enamel surface.

FIG. 11B and FIG. 11-C show fluorescence from 0 minutes to 20 minutes for OsteoSense 750 diluted 1:100 and diluted 1:10, respectively, and bound to caries on a demineralized enamel surfaces.

FIG. 11D shows fluorescence from 0 minutes to 15 minutes for IR820 diluted 1:10 and bound to caries on a demineralized enamel surface.

FIG. 12A and FIG. 12B are each a photograph of a tooth with a caries lesion that was contacted with a fluorescent probe using a syringe and imaged with a light microscope (FIG. 12A) and a fluorescent microscope (FIG. 12B). Comparison of the images shows that the fluorescent probe was delivered into and detected the area, size and depth of the caries lesion. A bar indicates 500 micrometers (μm).

FIG. 13 is a set of photographs showing each of: detection of fluorescence caused by reflection of a probe bound to a caries lesion on an adjacent tooth (panel A), placement of a separator to eliminate the reflection fluorescence on the adjacent tooth (panel B), and corresponding elimination by the separator of fluorescence caused by reflection of a probe bound to a caries lesion on an adjacent tooth (panel C).

FIG. 14 is a set of photographs showing a light microscope image of a tooth with a caries lesion (panel A), a fluorescent microscope image of the tooth having OsteoSense 750 bound to the caries lesion (panel B), and a fluorescent microscope image of the tooth three minutes (panel C) and 10 minutes (panel D) after adding hydrogen peroxide (38%, Ultradent Products Inc. South Jordan, Utah) to remove OsteoSense 750 from the tooth. Bars indicate 500 micrometers (μm) and one millimeter (mm).

FIG. 15A and FIG. 15B are photographs showing detection of fluorescence of OsteoSense 750 probe bound to caries lesions in human extracted teeth. The teeth were contacted with OsteoSense 750 probe, illuminated with near infrared light at 740 nm wavelength through the occlusal layer of enamel (FO; FIG. 15A), or at the interproximal area (FI; FIG. 15B), and detected for fluorescence emission.

FIG. 16A and FIG. 16B are photographs showing histological examination of teeth for caries and identified with a light microscope as having No Lesion (FIG. 16A) or Lesion (FIG. 16B). FIG. 16A shows a tooth with no caries lesion. FIG. 16B shows a tooth with a caries lesion that extends less than one-half of the depth of the enamel (FIG. 16B, left), and a tooth with a caries lesion that extends more than one-half of the depth of the enamel (FIG. 16B, right).

FIG. 17A and FIG. 17B are photographs showing a light microscope image of the depth of a caries lesion in a tooth (FIG. 17A), and a fluorescent microscope image of the same tooth having OsteoSense 750 bound to the caries (FIG. 17B). Comparison of the photographs shows that the fluorescent probe/optical detection method delineates the depth of the caries. Bars indicate 500 micrometers (μm).

FIG. 18A-FIG. 18C are photographs of a light microscope image of the actual depth of a caries lesion in a tooth (FIG. 18A), a fluorescent microscope image of OsteoSense 750 bound to the caries lesion (FIG. 18B), and a fluorescent microscope image of the tooth after adding hydrogen peroxide (38%, Ultradent Products Inc.) to remove OsteoSense 750 from the lesion (FIG. 18C).

FIG. 19A and FIG. 19B are line graphs showing amount of OsteoSense 750 released from samples of HA powder by incubation for 15 minutes at 37° C. with each of 1%, 5%, 10%, and 20% solutions of each of phosphoric acid (PA), sodium phosphate monobasic (M), sodium phosphate dibasic (D), and methylene phosphoric acid (MDP). Graphs show relative fluorescence units (RFU) either as a percent of OsteoSense 750 released (FIG. 19A) or as a percent of total OsteoSense 750 reference value (FIG. 19B), as a function of solution concentration.

FIG. 20A and FIG. 20B are line graphs showing amount of OsteoSense 750 released from HA powder by incubation at room temperature with each of 1%, 5%, 10%, and 20% solutions adjusted to pH 6.5 of each of phosphoric acid (PA), sodium phosphate monobasic (M), sodium phosphate dibasic (D), and methylene phosphoric acid (MDP). Graphs show RFU either as a percent of OsteoSense 750 released (FIG. 20A) or as a percent of total OsteoSense 750 reference value (FIG. 20B), as a function of concentration.

FIG. 21A and FIG. 21B are line graphs showing amount of OsteoSense 750 released from HA powder by incubation at room temperature with each of 1%, 5%, 10%, and 20% solutions of each of phosphoric acid (PA), sodium phosphate monobasic (M), sodium phosphate dibasic (D), and methylene phosphoric acid (MDP), to which 1% calcium chloride was added. Graphs show RFU either as a percent of OsteoSense 750 released (FIG. 21A) or as a percent of total OsteoSense 750 reference value (FIG. 21B), as a function of concentration of solution.

FIG. 22A and FIG. 22B are photographs showing an optical system for detecting bisphosphonate and pyrophosphonate bound to hydroxyapatite on a tooth.

FIG. 22A shows a computer adjacent to platform. The computer contains software for controlling the electronic components on the platform and for collecting the data. The platform includes the electronic components for detecting fluorescence emissions and photographing the tooth.

FIG. 22B shows a vertical view of the platform shown in FIG. 22A. The platform includes hardware and software for detecting bisphosphonate and/or pyrophosphonate bound to hydroxyapatite on a tooth including: an electrical stage, optical fibers, a light source, and a charge coupled device (CCD) camera.

FIG. 23 is a drawing showing a system and methods for detecting enhancement of remineralization of a tooth of a subject and/or detecting inhibition of a caries progression or loss of hydroxyapatite. The drawing of the system includes: optical fibers that produce and direct electromagnetic light waves at specific wavelengths including 730 nm or 780 nm, or 890 nm, 910 nm, or 940 nm; a stand or mount for a tooth sample that is illuminated by the optical fibers; and a CCD camera that captures the image of the tooth and/or fluorescence emissions from a fluorescent dye or tooth.

FIG. 24A-FIG. 24D are photographs showing a light microscope image and fluorescent microscope images of a tooth having a caries. The tooth was contacted with OsteoSense 750 which is a bisphosphonate bound to a fluorescent cyanine dye.

FIG. 24 shows a light microscope image of the depth of a caries lesion in a tooth.

FIG. 24B shows a fluorescent microscope image of the same tooth having a fluorescent cyanine dye (OsteoSense 750) bound to the caries (FIG. 24B). The fluorescent microscope image was obtained by, illuminating the tooth with 740 nm wavelength excitation light, and detecting emission light using a wavelength filter at 780 nm.

FIG. 24C shows the size of a caries (A) indicated by a light microscope.

FIG. 24D shows the size of a caries (B) bound to a fluorescent cyanine dye and delineated by a fluorescent microscope. Comparison of the photographs shows that the fluorescent probe/optical detection method indicates depth of the caries. Bars are 500 micrometers (μm).

FIG. 25A-FIG. 25D are photographs showing that bisphosphonate binds to hydroxyapatite in an interproximal caries lesion. A tooth specimen was sectioned to expose the interproximal caries lesion, and the section surface was varnished with nail polish. A solution of OsteoSense 750 (1:100 dilution) was then applied to the interproximal caries lesion.

FIG. 25A shows a light microscope image of the tooth with the interproximal caries lesion.

FIG. 25B shows a fluorescent microscope image of the tooth having OsteoSense 750 bound to the caries lesion. The caries lesion was then applied three times with OsteoSense 750 (1:30 dilution). Then a hydrogen peroxide solution (38% Opalescence Boost Teeth Whitening; Ultradent Inc., South Jordan, Utah) was applied to the caries lesion to quench the fluorescence.

FIG. 25C shows a fluorescent microscope image of the tooth after the hydrogen peroxide was applied as described in FIG. 25B above. Data show that the fluorescence of the bisphosphonate-fluorescent dye was quenched, however the bisphosphonate remained in the caries lesion.

FIG. 25D shows a fluorescent microscope image of the tooth after a solution of OsteoSense 750 (1:100 dilution) was then applied to the interproximal caries lesion and after the fluorescent image was taken in FIG. 25C. The image shows only weak fluorescence.

An excitation light of 740 nm excitation light and an emission filter at 780 nm was used in FIGS. 25B, C and D for the fluorescence imaging. Bars indicate 500 μm.

FIG. 26A-FIG. 26D are photographs showing that bisphosphonate binds to hydroxyapatite in an interproximal caries lesion. A tooth specimen was sectioned to expose the interproximal caries lesion, and the section surface was varnished with nail polish. A solution of OsteoSense 750 (1:100 dilution), a bisphosphonate attached to a fluorescent dye, was then applied to the interproximal caries lesion.

FIG. 26A shows a light microscope image of the tooth with the interproximal caries lesion.

FIG. 26B shows a fluorescent microscope image of the tooth having OsteoSense 750 bound to the caries lesion. The caries lesion was then applied three times with OsteoSense 750 (1:30 dilution). Then a hydrogen peroxide solution (38% Opalescence Boost Teeth Whitening; Ultradent Inc.) was applied to the caries lesion to quench the fluorescence.

FIG. 26C shows a fluorescent microscope image of the tooth after the hydrogen peroxide was applied as described in FIG. 26B above. Data show that the fluorescence of the bisphosphonate-fluorescent dye was quenched, however the bisphosphonate remained in the caries lesion.

FIG. 26D shows a fluorescent microscope image of the tooth after a solution of OsteoSense 750 (1:100 dilution) was then applied to the interproximal caries lesion and after the fluorescent image was taken in FIG. 26C. The image shows only weak fluorescence.

An excitation light of 740 nm excitation light and an emission filter at 780 nm was used in FIG. 26B, FIG. 26C and FIG. 26D for the fluorescence imaging. Bars indicate 500 μm.

FIG. 27A-FIG. 27D are photographs showing that bisphosphonate binds to hydroxyapatite in an occlusal caries lesion. A tooth specimen was sectioned to expose the occlusal caries lesion, and the section surface was varnished with nail polish. A solution of OsteoSense 750 (1:100 dilution), a bisphosphonate attached to a fluorescent dye, was then applied to the occlusal caries lesion.

FIG. 27A shows a light microscope image of the tooth with the interproximal caries lesion.

FIG. 27B shows a fluorescent microscope image of the tooth having OsteoSense 750 bound to the caries lesion. The caries lesion was then applied three times with OsteoSense 750 (1:30 dilution). Then a hydrogen peroxide solution (38% Opalescence Boost Teeth Whitening; Ultradent Inc.) was applied to the caries lesion to quench the fluorescence.

FIG. 27C shows a fluorescent microscope image of the tooth after the hydrogen peroxide was applied as described in FIG. 27B above. Data show that the fluorescence of the bisphosphonate-fluorescent dye was quenched, however the bisphosphonate remained in the caries lesion.

FIG. 27D shows a fluorescent microscope image of the tooth after a solution of OsteoSense 750 (1:100 dilution) was then applied to the interproximal caries lesion and after the fluorescent image was taken in FIG. 27C. The image shows only weak fluorescence.

An excitation light of 740 nm excitation light and an emission filter at 780 nm was used in FIG. 27B, FIG. 27C and FIG. 27D for the fluorescence imaging. Bars indicate 500 μm.

FIG. 28A-FIG. 28C are a set of photographs showing fluorescence emission of OsteoSense 750 bound to interproximal caries lesions in each of three teeth (FIG. 28A, FIG. 28B and FIG. 28C). The images were obtained prior to (left column) and after (right column) treatment with OsteoSense 750 (1:30 dilution). For each tooth the extent of fluorescence emission detected after treatment with OsteoSense 750 was much weaker than the fluorescence emission detected prior to treatment with OsteoSense 750.

FIG. 29A and FIG. 29B are graphs showing intensity on the ordinate as a function of autofluorescence of enamel and dentin, and excitation and emission wavelengths of OsteoSense 750 and a NIR bisphosphonate derivative described herein. The bisphosphonate derivative described herein has improved optical imaging of early stage dental caries compared to OsteoSense 750, and illumination of the bisphosphonate derivative results in emission that has little or no overlap in wavelength with the wavelength of autofluorescence of enamel and dentin.

FIG. 29A shows autofluorescence of tooth enamel and dentin (400 nm-about 800 nm), and excitation wavelength and emission wavelengths of OsteoSense 750 (730 nm and 750 nm respectively). Illumination of teeth using wavelengths in the near infrared (NIR) range excites the autofluorescence of the enamel and dentin, which overlaps the wavelengths of OsteoSense 750 emission and excitation.

FIG. 29B shows that the autofluorescence of tooth enamel and dentin (400 nm to 800 nm), and the excitation wavelength (about 810 nm) and emission wavelength (about 830 nm) of a new bisphosphonate derivative do not overlap wavelengths of autofluorescence of enamel and dentin.

FIG. 30A-FIG. 30C are photographs showing fluorescence emission of OsteoSense 750 bound to interproximal caries lesions in a decayed teeth (FIG. 30A-FIG. 30C). The images were obtained by illuminating the interproximal caries lesions and detecting fluorescence emissions from the buccal side of the tooth.

FIG. 31A-FIG. 31C are photographs showing fluorescence emission of OsteoSense 800 bound to enamel caries lesions in each of three teeth (FIG. 31A, FIG. 31B and FIG. 31C). The teeth were contacted with OsteoSense 800 probe, and were illuminated at 780 nm. Fluorescence emissions at 800 nm were detected. Comparison of the fluorescence data in this figure for OsteoSense 800 to fluorescence data for OsteoSense 750 in FIG. 30A-FIG. 30C shows greater fluorescence emission and image clarity for teeth treated with OsteoSense 800 compared to teeth administered. OsteoSense 750.

FIG. 32 is a set of photographs showing fluorescence emission of OsteoSense 800 bound to enamel caries lesions in a sectioned specimen (FIG. 32 left) of a tooth and the whole tooth (FIG. 32 right). The tooth was contacted with OsteoSense 800 probe and illuminated at 780 nm. Fluorescence emission wavelengths at 800 nm were detected.

FIG. 32 left shows fluorescence emission of a whole tooth having a caries lesion bound to OsteoSense 800 probe.

FIG. 32 right shows fluorescence emission of a section (one millimeter, m) of the tooth in FIG. 32 left. FIG. 32 right shows a magnified image of the size and location of the caries lesion bound to the OsteoSense 800 probe.

FIG. 33 is a drawing of a chemical reaction of indocyanine green sulfo N-hydroxysuccinimide ester (ICG-SulfoNHS) with disodium pamidronate in the presence of a 3-hydroxypropionitrile to yield an indocyanine green pamidronate derivative.

FIG. 34A-FIG. 34C are photographs showing enamel caries lesions in tooth sections viewed using histological examination, the fluorescence probe system described herein, and an Xradia MicroXCT 3D X-ray microscope. A tooth having a caries lesion was examined at two different magnification levels using the Xradia MicroXCT 3D X-ray microscope. The tooth specimen was sectioned to expose the interproximal caries lesion and histological examined. A solution of OsteoSense 750 was applied to the interproximal caries lesion, the section was illuminated with NIR light and the fluorescence emission was detected. Data show that the fluorescent probe and MicroXCT 3D X-ray microscope effectively identified the early stage caries lesion. Bars indicate 1000 micrometers (microns, μm).

FIG. 34A shows a light microscope image of the tooth section with the interproximal caries lesion.

FIG. 34B shows a fluorescent microscope image of the tooth having OsteoSense 750 bound to the caries lesion.

FIG. 34C shows a image obtained using the Xradia MicroXCT 3D X-ray microscope. Examination of the image shows that Xradia system images the tooth using high resolution (to one micrometer) without having to section the tooth.

FIG. 35A-FIG. 35C are photographs showing enamel caries lesions (100 micrometers in size) identified using a light microscope and an Xradia MicroXCT 3D X-ray microscope. Data show that the Xradia MicroXCT 3D X-ray microscope or histological examination, identified the caries lesions. Bar indicates 5000 micrometers (μm).

FIG. 35A shows a light microscope image of the interproximal caries lesion.

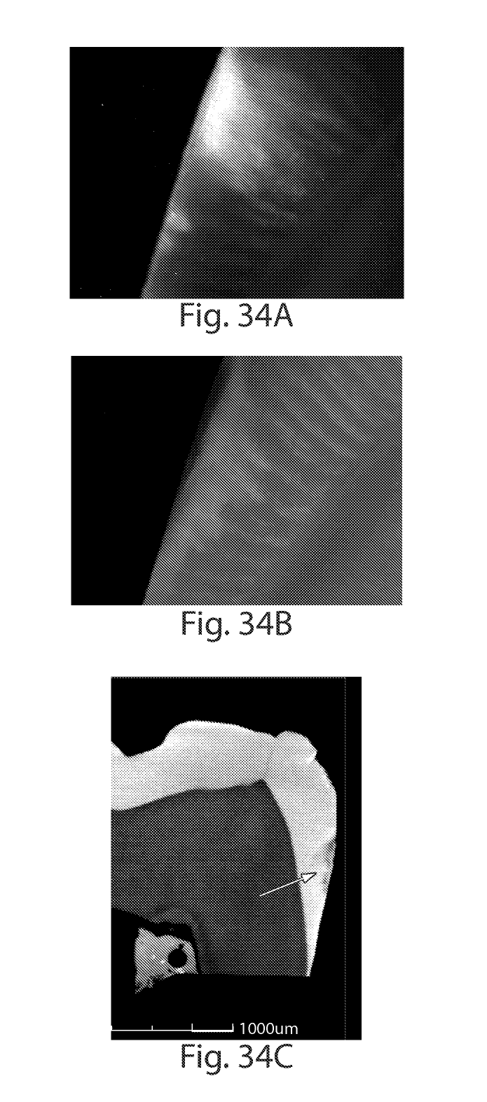

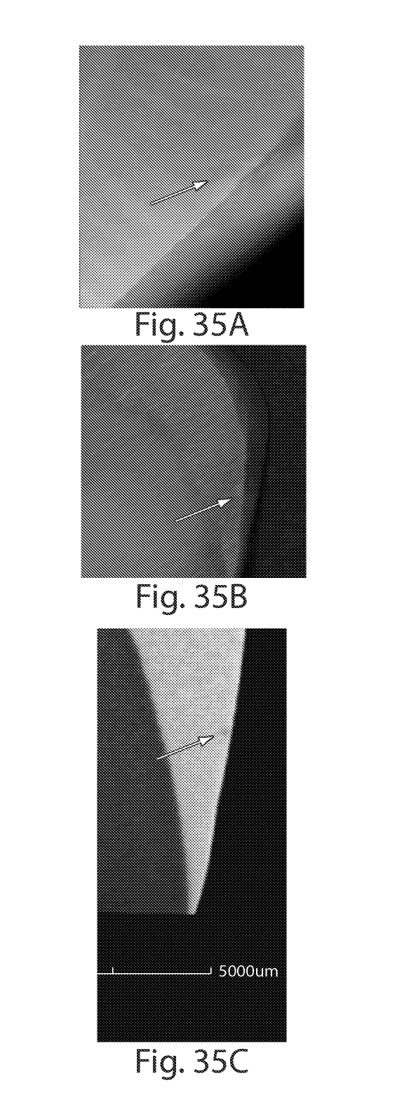

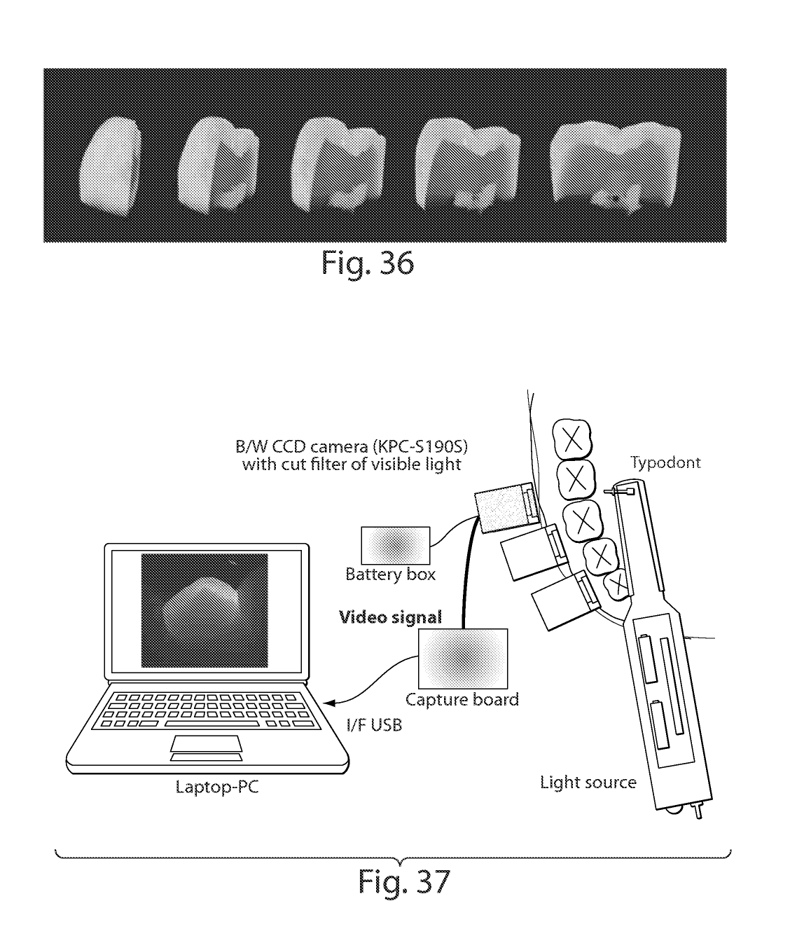

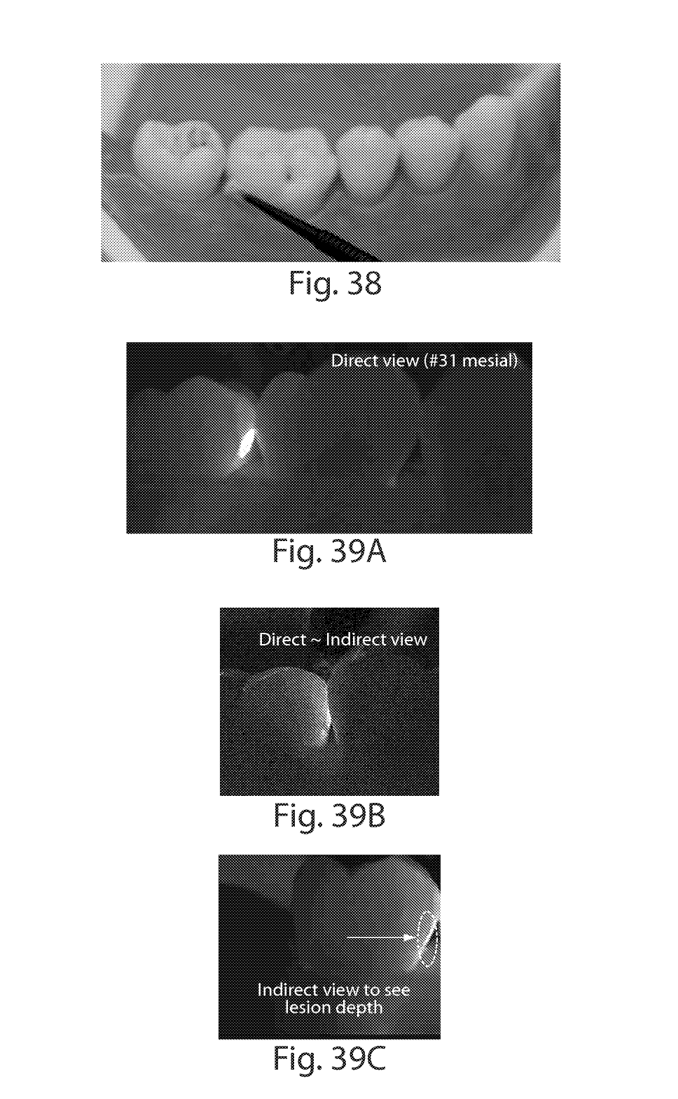

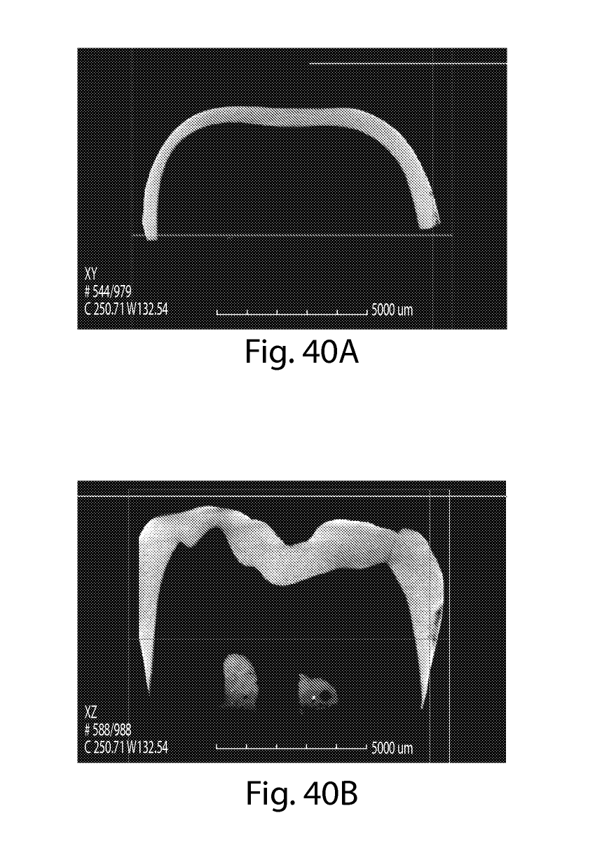

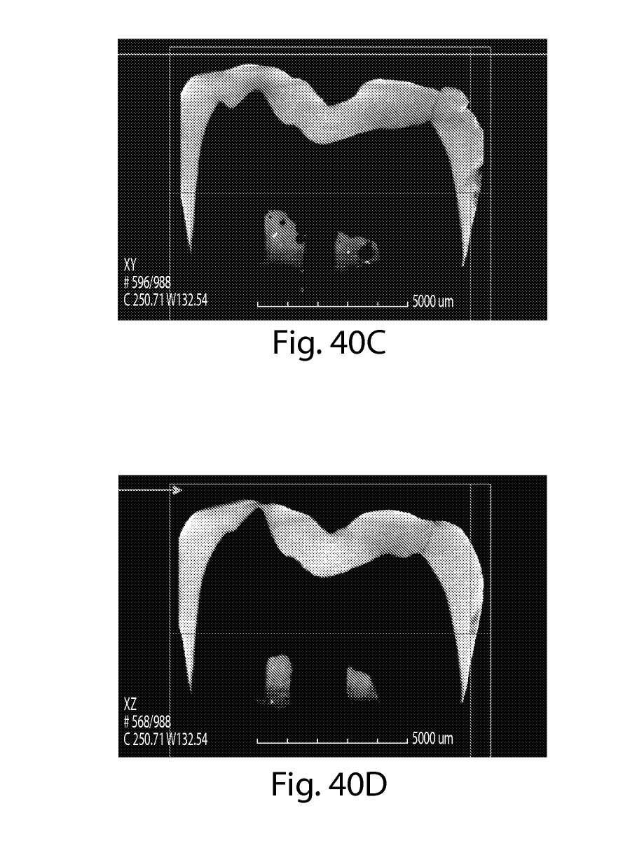

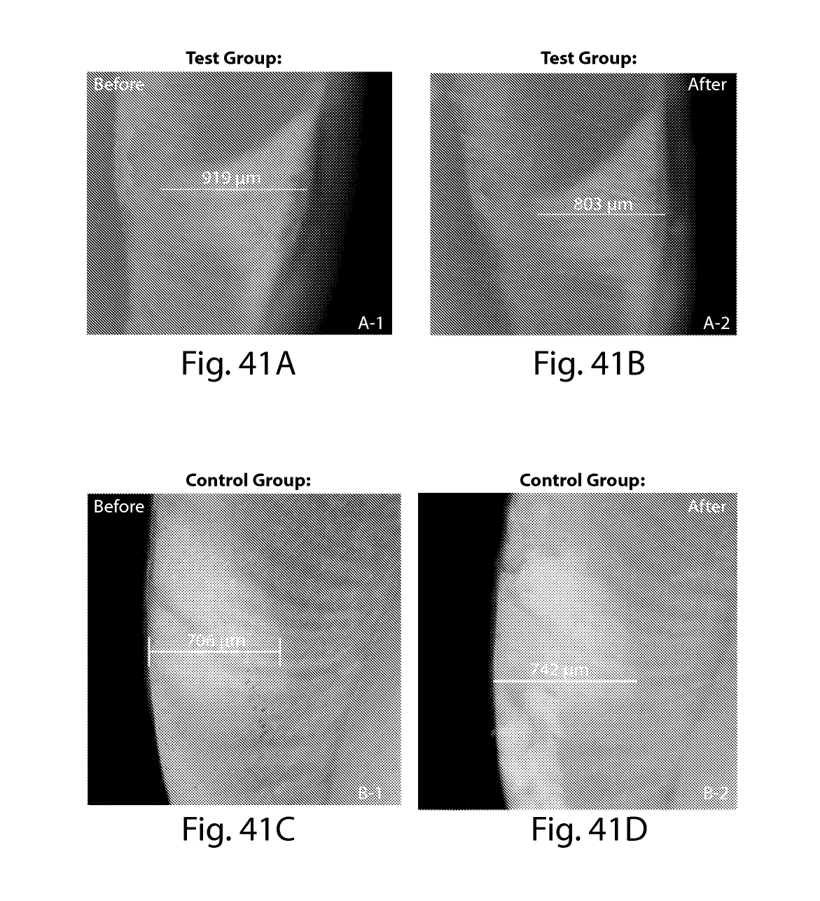

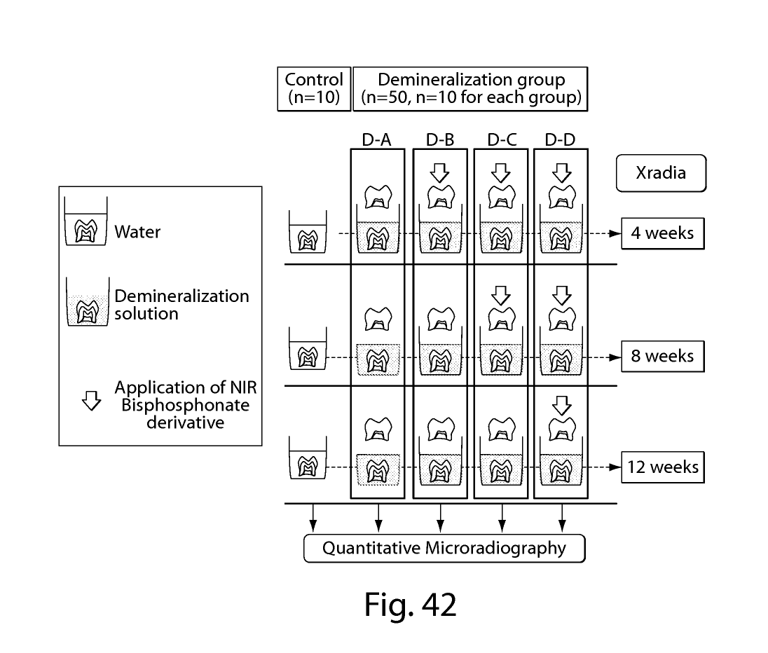

FIG. 34B shows a magnified light microscope image of the caries lesion shown in FIG. 35A.

FIG. 35C shows an image of the caries lesion obtained using the Xradia MicroXCT 3D X-ray microscope.

FIG. 36 is a set of photographs of three-dimensional images of an enamel caries lesion using the Xradia MicroXCT 3D X-ray microscope. Data show that the Xradia MicroXCT 3D X-ray microscope was effective for viewing the presence and size of the caries lesion without having to section the tooth.

FIG. 37 is a drawing of a system for caries detection and remineralization. A typodont, which is a model of the oral cavity including gingiva and the teeth, is illuminated (e.g., on the interproximal space, occlusal surface, and lingual surface) with wavelengths of light from a light source. A B/W CCD camera with cut filter of visible light for detecting an emission wavelength of light and/or an image of the tooth is connected to a battery box. The capture board digitizes an incoming stream of video signal and data (e.g., analogue) and is connected to a laptop computer or personal computer (PC) using an implementers forum-certified universal serial bus (I/F USB) hub or connector. The video signal is sent to the computer and analyzed and visualized.

FIG. 38 is a photograph of application of an NIR fluorescent bisphosphonate compound/derivative to a tooth (e.g., interproximal space and buccal surface) in a typodont using a microbrush. The NIR fluorescent bisphosphonate derivative binds to exposed/decayed tooth surfaces of the tooth and does not bind to intact tooth surfaces and soft tissue (silica gingival) of the typodont. The typodont is rinsed with water after application of the NIR bisphosphonate derivative.

FIG. 39A-FIG. 39C are photographs showing OsteoSense 800 bound to an interproximal caries lesion on a tooth. The photographs were taken from different positions and views using a CCD camera.

FIG. 39A is a photograph showing a mesial (direction towards the anterior midline in a dental arch) view of the fluorescence of OsteoSense 800 probe bound to an early stage caries lesion in a tooth.

FIG. 39B is a photograph showing a distal (direction towards the last tooth in each quadrant of the mouth) view of the fluorescence of OsteoSense 800 probe bound to an early stage caries lesion in a tooth.

FIG. 39C is a photograph showing an indirect view of the fluorescence of a OsteoSense 800 probe bound to an early stage caries lesion in a tooth. The indirect image was obtained by placing the CCD camera perpendicular to the interproximal area. The depth of the caries lesion is seen using this indirect view.

FIG. 40A-FIG. 40D are images of a caries lesion in the interproximal area of the tooth using a three-dimensional scan using an Xradia MicroXCT 3D X-ray microscope. The tooth imaged is the tooth shown in FIG. 34. Three-dimensional imaging of the interproximal area was determined using the Xradia MicroXCT 3D X-ray microscope shows the true depth and size of the caries lesion.

FIG. 41A-FIG. 41D are photographs showing that methylene disphosphonate (MDP) remineralized decayed teeth.

FIG. 41A shows a light microscope image of a demineralized carious tooth contacted with MDP. Bar indicates 919 μm.

FIG. 41B shows a light microscope image of a demineralized carious tooth contacted with MDP after further demineralization with a demineralization solution. Data show that the MDP treatment inhibited further demineralization of the tooth and in fact remineralized the decayed tooth approximately 13%. Bar indicates 803 μm.

FIG. 41C shows a light microscope image of a control carious tooth contacted with water. Bar indicates 706 μm.

FIG. 41D shows a light microscope image of the demineralized control tooth in FIG. 41C after further demineralization with a demineralization solution. Data show that without MDP treatment the caries lesion was demineralized and the size of the caries increased. Bar indicates 742 μm.

FIG. 42 panel is a drawing of a protocol for determining that an NIR bisphosphonate derivative is effective for protection again demineralization and promotion of remineralization of a decayed tooth. Sets of teeth (n=50) are contacted with 1.0 μl of an NIR fluorescent bisphosphonate derivative (downward arrow), or with water (control) or a mixture of these. The sets of teeth are then incubated for a period of time (weeks) in either water (clear) or a demineralization solution (shaded), and then imaged using a Xradia MicroXCT 3D X-ray microscope and quantitative microradiography.

### DETAILED DESCRIPTION

Dental caries remain a serious chronic disease even though prevalence of dental caries has been declining in the United States in the last few decades (Brown L. J. et al. 2000 J Am Dent Assoc 131(2): 223-31). Untreated dental decay leads to progressive gum disease, loss of teeth and its subsequent negative effects on nutrition, education and social interactions, and systemic infections, with death as the worst-case final outcome. Dental decay is a particular problem in low-income populations, especially in children. The World Oral Health Report 2003 published by the WHO, indicates that dental caries is a major health problem in most industrialized countries, affecting 60%-90% of school children and most adults (Petersen, P. E. 2003 The World Oral Health Report 2003: Continuous Improvement of Oral Health in the 21st Century—The Approach of the WHO Global Oral Health Programme World Health Organization, Geneva). Dental decay is one of the most common and also most easily preventable infectious diseases of childhood. Many caries are undetected with low access to dental care, and progression reaches a stage at which surgical restoration or extraction is the only available treatment. Dental restorations are vulnerable to recurrent caries, technical deficiencies and material failures, and traditional dental restorative techniques generate an unfavorable cycle of increasing tooth destruction, sepsis and even death (Gil M. et al. Assessment of incipient interproximal caries diagnosis with new optical technology, IADR/AADR/CADR 88th General Session and Exhibition abstract #2822; Khorashadi S. et al. Detection of incipient interproximal legions using dye enhanced fluorescence, IADR/AADR/CADR 88th General Session and Exhibition abstract #2821); Gil M. et al. Infiltration of near InfraRed Fluorescent Dye in Enamel Lesions, IADR/AADR/CADR 89th General Session and Exhibition abstract #3744; and Nagai S. et al. Assessment of a Bisphosphonate Compound for Inhibition of Caries. IADR/AADR/CADR 90th General Session and Exhibition abstract #1275). Therefore, there is an urgent need for a reliable method for early detection and active preventive care (McComb D. 2005 Dent Clin North Am 49:847-865). A paradigm of operative conservatism, minimally invasive dentistry, incorporates detecting, diagnosing, intercepting and treating dental caries at the microscopic level (Rainey J. T. 2002 Dent Clin North Am 46(2): 185-209) Expanding the range of sensitive and early caries detection would greatly help in achieving a goal of operative conservatism. What is needed is a safe non-radiographic method that is sufficiently sensitive to consistently and reliably detect very small early-stage carious lesions. Such a method would provide early diagnosis and patient-tailored active preventive therapy (Nyvad B. 2004 Caries Res. 38: 192-198).

A critical unmet need exists for an optical caries detection system that can detect and treat early interproximal caries. Despite considerable effort to develop new methods to detect very early lesions, the current standard for caries detection is visual-tactile examination and X-rays (Celiberti P. et al. 2010 J Dentistry 38: 666-670; Louie T. et al. 2010 Lasers Surg Med. 42(10): 738-745; Staninec M. et al. 2010 Lasers Surg Med. 42(4): 292-298; Fried D. et al. 2005 Dent Clin North Am. 49(4): 771-793; Eggertssona H. 1999 et al. Caries Res. 33: 227-233; and van de Rijke J. W. et al. 1991 Caries Res 25: 335-340). However, X-ray methods detect only advanced lesions with a depth of at least 500 μm in enamel, leaving incipient caries undetected (Stookey G. K. et al. 2005 Dent Clin North Am. 49(4): 753-770). Furthermore, because of the location, interproximal caries are detected only radiographically, and X-ray images usually significantly underestimate actual size or depth of a carious lesion (White S. C. et al. 2003 Oral Radiology: Principles and Interpretation 5th edition Mosby Inc.). By the time a lesion is visible on an X-ray bitewing, only invasive class II restoration is effective, subjecting the tooth to a lifetime of treatment. Conventional X-ray bitewing procedures expose the patient to ionizing radiation. The collective doses to patients from the medical use of ionizing radiation increase significantly each year, and the cumulative risk of cancer at age 75 from diagnostic X-rays is directly linked to the annual exposure frequency (Berrington de Gonzalez A. et al. 2004 Lancet 363: 345-351). Numerous methods are practiced for limiting X-ray exposure in most hospital settings, and doses generally administered are as low as reasonably achievable (ALARA). However clear quantitative guidance for the risk-benefits of modern X-ray imaging methods is still lacking (Moores B. M. et al. 2011 Radiat Prot Dosimetry 147(1-2): 22-29). Thus, an innovative approach is still needed for early caries detection that outperforms the current modality without exposing patients to ionizing X-ray radiation.

Commercially available optical methods for visual-tactile examination and radiographs for caries detection include digital imaging under transillumination, quantitative light-induced fluorescence, and laser fluorescence (Schneiderman A. et al. 1997 Caries Res 31:103-110; Heinrich-Weltzien R. et al. 2003 Quintessence Int 34(3): 181-188; and Shi X. et al. 2000 Caries Res 34:151-158). Optical diagnostic tools exploit changes in light scattering. The digital imaging fiber-optic transillumination (DIFOTI) device uses visible light, which does not penetrate deep enough into enamel to detect small lesions. Quantitative laser/light-induced fluorescence (QLF) takes advantage of the empirical relationship between overall lesion demineralization and the loss of autofluorescence to detect early caries. However the QLF equipment is costly and generally purchased only by universities and research centers (Heinrich-Weltzien R. et al. 2003 Quintessence Int 34(3): 181-188). A more recent technology, DIAGNOdent (Kayo Dental; Biberach/Riss, Germany), is designed to detect Near Infrared (NIR) fluorescence from bacterial porphyrins. However, this device detects caries only after a sufficient amount of bacterial by-products has accumulated at the site, having reported sensitivity of only 40% (Shi X. et al. 2000 Caries Res 34:151-158). None of these devices meet criteria for routine screening for routine and inexpensive detection of early-stage interproximal caries.

Compositions, methods and kits described herein are used to treat early stage caries by inhibiting further demineralization and caries progression. However this property of bisphosphonate not heretofore been investigated in dentistry for treatment of dental caries. Synthesis, characterization and testing NIR fluorescence bisphosphonate derivative are shown in Examples herein to be useful for inhibition enamel dissolution and caries progression. A new caries detection system using these bisphosphonate derivatives is shown in Examples herein to detect early interproximal caries with high sensitivity. The commercially available NIR fluorescence bisphosphonate derivative, OsteoSense 750, was observed herein to be effective in detecting incipient interproximal caries. Without being limited by any particular theory or mechanism of action, it is envisioned that caries detection using NIR probes is improved by minimizing an overlap in wavelengths of the fluorescence peak of the bisphosphonate probe and enamel autofluorescence which is 400-800 nm. Methods for synthesizing effective probe imaging agents are shown and these probes are safer and more sensitive than traditional diagnostic radiography.

The early caries detection system described herein is more sensitive than X-rays and avoids exposing a subject to unnecessary exposure of ionizing radiation. This detection system used NIR fluorescent bisphosphonate derivative probes and NIR transillumination to effectively bind to and detect early stage dental caries. Industry has used optical imaging for real-time, non-radiographic and high-resolution imaging of fluorophores embedded in diseased tissues (Zaheer A. et al. 2001 Nature Biotechnology 19: 1148-1154; Bhushan J. K. et al. 2008 J Am Chem. Soc. 130: 17684-17694; Frangioni J. V. 2007 Current Opinion in Biotechnology 18: 17-25; and Zaheer A. et al. 2006 Vasc Biol. 26: 1132-1136). Without being limited by any particular theory or mechanism of action, it is envisioned that NIR (700-900 nm) fluorescence-based imaging is especially attractive for noninvasive in vivo imaging because of the relatively low tissue absorption, scatter, and minimal autofluorescence of body tissues in the NIR emission wavelength range. Methods and systems described herein used NIR illumination and NIR probes to detect early interproximal caries.

The early caries detection methods and systems described herein include a fluorescent probe that targets exposed dental hydroxyapatite; and the methods and system used wavelengths of fluorescence excitation and emission that were found to be distinct and sufficiently separate from the autofluorescence of dental enamel and dentin in the visible range of the electromagnetic spectrum. Autofluorescence of enamel and dentin refers to emission in the green part of the visible spectrum from teeth illuminated with light (e.g., high intensity blue light). Autofluorescence wavelengths that overlap a probe excitation and emission wavelength complicate analysis and detection of the probe bound to the caries, resulting in a very complex and problematic resolution due to non-specific fluorescence emission. The probe in certain embodiments described herein was characterized by wavelength excitation and emission that were found to not overlap with the autofluorescence wavelengths of enamel and dentin, allowing for effective fluorescence visualization of the probe bound to a caries without accompanying fluorescence of the enamel and dentin.

Examples herein show a probe (e.g., bisphosphonate and pyrophosphonate) with a fluorescent tag such as ICG (having an emission wavelength peak of about 850 nm) that is excitable in the NIR range. The NIR probe in certain embodiments is a fluorescent bisphosphonate derivative that has high affinity for exposed hydroxyapatite and that possesses optimized excitation and emission wavelengths that are distinct from the range of the autofluorescence wavelengths of enamel and dentin. The NIR bisphosphonate derivative is shown in examples herein to have been effective for detection of early stage caries. Examples shows methods for enhancing remineralization of a tooth of a subject to inhibit caries progression, the method including contacting the tooth or oral cavity of the subject with a composition including at least one component selected from a bisphosphonate and/or a pyrophosphonate such that the composition infiltrates an area of the tooth having the caries lesion to reduce porous hydroxyapatite and/or inhibit caries progression or loss of hydroxyapatite, compared to a tooth not so contacted. The composition optionally comprising an indicator, stain or dye for imaging the bisphosphonate and/or pyrophosphonate bound to the expose hydroxyapatite, and to indicate optionally the need to apply additional amounts of the composition to remineralize the tooth.

Compositions, methods and kits described herein also include application of the NIR fluorescence imaging bisphosphonate derivative to promote remineralization of dental tissues such as enamel and dentin. A constant dynamic cycle exists between dental demineralization and dental remineralization, however the process of remineralization is far slower than that of demineralization. The slower rate of remineralization results in a tooth caries over time progressing deeper into the tooth causing more advanced decay. Thus there is a need for invasive dental treatments to prevent advancing tooth decay. Examples herein show a bisphosphonate derivative having a NIR fluorescent tag that binds to the dental enamel and/or dentin and inhibits the progression of caries in the tooth. Bisphosphonates described herein have a very high binding affinity to hydroxyapatite and slow the rate and extent of tooth demineralization. Bisphosphonates (also called diphosphonates) have therapeutic potential for preventive dentistry similar to fluoride ions (F−) by effectively inhibiting enamel dissolution and cavitation. The bisphosphonates in addition are effective caries diagnostic agents. Examples herein show that the bisphosphonate derivatives MHDP (methane-hydroxy-diphosphonate) and EHDP (ethane-1-hydroxy-1,1-diphosphonate) effectively protect dental enamel surfaces against demineralizing conditions such as lactic acid by competitively blocking the exposed surface calcium sites (De Rooij J. E. et al. 1984 J Dent Res 63: 864-867; Christoffersen J. et al. 1991 J Dent Res 70: 123-126; and Budz J. A. et al. 1988 J Dent Res 67: 1493-1498).

Detecting early interproximal lesions still poses a challenge in clinical dentistry today. Invasive restorative treatment is often necessary by the time carious lesions are detectable by bitewing radiographs as shown in Brown L. J. et al., 2000 J Am Dent Assoc 131(2): 223-31, and Hannigan A. et al., 2000 Caries Res 34: 103-108. Therefore, a more sensitive method for detecting early interproximal lesions would provide an opportunity for active preventive measures. Although there are many parallel efforts to detect early caries, devices fail to detect incipient interproximal lesions reliably (Hall A. et al., 2004 J Dent Res 83 (Spec Iss C): C89-C94; Heinrich-Weltzien R. et al., 2003 Quintessence Int 34(3): 181-188; and Stookey G. K., 2005 Dent Clin North Am. 49(4): 753-770).

While the current industry-accepted standard for caries detection is a combination of clinical and radiographic examination, the intra-oral optical device has potential to detect a very small optical property change which is missed by the human eye. Such a development would spare patients from exposure to the radiation associated with radiographs, could reduce labor-intensive clinician time in detecting very early stage caries, and make detection possible for caries that might otherwise go undetected until a later stage.

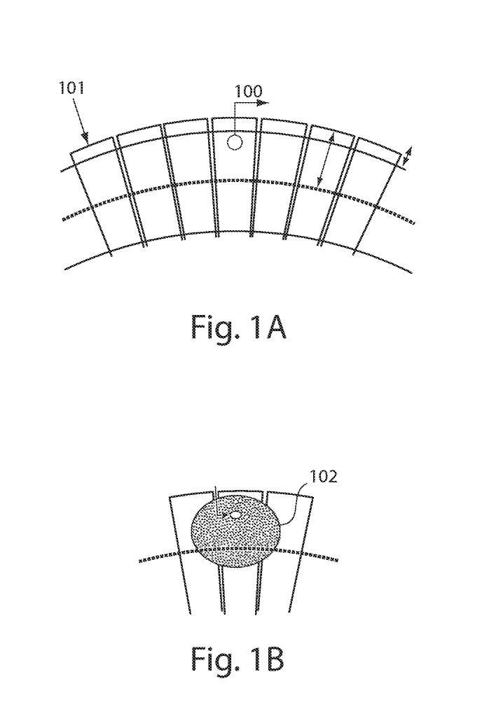

Demineralization on an enamel surface of a tooth results from the presence in the oral cavity of acid and bacteria, and these agents initiate dental caries. FIG. 1A shows that interaction/invasion of acid/bacteria into tooth enamel (i.e., enamel prism) produces a white spot (100) caries lesion that is not detected using conventional X-rays. Hence a molecule capable of binding as a positive/negative ion or by electrovalent bonds in the region of an early stage caries, i.e., an area of demineralization, is here envisioned without limitation as a potential probe for binding to early stage caries.

Examples herein show classes of useful probes that have detectable properties with respect to light: probes that fluoresce in excitation light illumination; probes that generate fluorescence without excitation by light, viz., bioluminescent probes such as the system of luciferase and luciferin; and probes that absorb illuminating light such as Bismuth, Gold colloid.

A slight difference of at least one of the standard optical properties (pattern of reflectance curves, color readings, Scattering/Absorption coefficients, or any other optical data) was sought herein in order to distinguish a caries lesion, from a sound tooth structure, and a handheld unit is programmed such that it is designed to take accurate optical properties of caries lesions in patients' oral cavities. Surprisingly, these differences were detected experimentally using the classes of probes described in examples herein. Therefore, using an optical device to detect potential changes in early lesions, such as an extent of binding of a probe capable of fluorescence or biofluorescence using another class of agent, is described herein as a tool for detecting early stage caries.

The wavelength of illumination (excitation) and emission for fluorescence probes were optimized for each probe. For example the excitation and emission fluorescence for HiLyte Fluor is about 720 nm to about 750 nm and is about 750 nm to about 800 nm, respectively. Data for additional probes are readily determined without undue experimentation according to the methods shown in the present application.

A standard optical device is modified for suitable use in the methods and compositions provided in the present application. Illuminating light was used on an entire tooth surface, for example, by scanning the surface with the beam of illuminating light. Scanning was particularly important for detection of presence of a caries lesion in an interproximal area. Further the examples herein show that angle of illumination was also important, such that illumination in an orientation parallel to or perpendicular to the direction of the enamel prism was particularly effective in detection of probe. In addition, examples herein showed that illumination directly through the occlusal layer of the enamel or to an interproximal area or adjacent to an interproximal area was also effective. Thus, the present invention provides methods for detecting an early stage dental caries in a subject involving contacting a tooth to determine presence of a caries lesion, selective binding of an optically detectable probe to the caries; such that the probe includes a molecule capable of binding the caries; and detecting the caries having bound probe using an optical device. It will be appreciated that the methods shown in the present application involved illuminating the tooth surface by a number of methods shown herein.

Among the optical device used with the various probes, detection was well obtained using a camera, for example, a small camera such as a fiber optic camera. Additional criteria with respect to the optical device was the size of diameter of beam of illuminating light that directly contacted the tooth (a smaller beam generated superior data); diameter of camera for detection of probes that absorbed light (a smaller diameter was superior, similarly to considerations of use of an endoscope); diameter of camera for detection of probes that were fluorescent (smaller was superior, similarly to considerations involved in use of an endoscope); and, illumination for scanning and a camera capable of recording light emissions in the scanning light.

Studies of caries detection indicated a lack of precise optical methods to detect an interproximal caries lesion. Although detection based on near infrared (NIR) was found to capture images of an interproximal lesion, a substantial number of false positive hits were found, and therefore this approach remains far from clinical use. However, compositions are shown in examples herein that bind specifically to caries and are detected using an optical device.

The phrase, a “white spot” (100) as used herein refers to a very early stage of decay that starts within enamel, for example within about 100 μm to 150 μm or less of the surface of the enamel. See FIG. 1A and FIG. 1B. White spot (100) as used herein refers to a stage and size of an early caries lesion. This caries under different conditions is visualized as gray, silver, white, brown, yellow or even translucent.

Bacteria and acid penetrate through the space between (10 μm) enamel prism (101). Demineralization initiated in these areas proceeds towards the surface of enamel. In general, X-ray imaging identifies only a caries lesion (102) having a depth of more than one-half of the enamel layer, so a white spot as defined herein is not detected by X-rays. Further, X-rays have an additional limitation of showing only about 60-70% of the actual extent of a caries. It is envisioned herein that detection of a white spot at an early stage of a lesion, using the methods and techniques herein provides subsequent possibilities for remineralization, and hence a cure for early stage caries. These treatments have potential to substantially improve dental health and lead to reduction of costs.

As used herein, the word “probe” refers to a detectable compound that specifically or preferentially binds a caries lesion. The term includes without limitation a molecule, a stain, a marker, and a dye capable of binding to a caries in the enamel layer of a tooth. In certain embodiments, the probe is a fluorescent composition, for example, tetracycline, HiLyte Fluor, Qdot, OsteoSense 750, Cy7, CardioGreen (ICG), IR820 (ICG), Far-Green two, AngioSense 750, Genhance 750, AngioSpark, 750, Alexa Fluor 750, Indocyanine Green, Doxorubicin, Riboflavin, Chlorophyll A, bacterial Chlorophyll and Porphyrin.

In general the fluorescent compound bound to enamel is detected by illuminating the treated tooth at an excitation wavelength, and detecting an area of light emission at an emission wavelength. In an alternative embodiment, the probe is a bioluminescent compound, for example, using luciferase to detect the compound luciferin or aequorin.

Alternatively, the probe is a composition that absorbs light, for example, bismuth, or colloidal gold. In general, light absorbent compositions are detected by illuminating an area of interest, for example, a tooth with a caries lesion, and detecting an area or region of the tooth that absorbs a specific wavelength of light, such as, absorbance of near infra red (NIR) light.

Gold nanoparticles have been designed that strongly absorb light in the NIR as shown in Gobin et al., Lasers in Surgery and Medicine 37: 123-129 (2005). The gold nanoparticles were used with NIR to provide solder welds in wound-healing research, known as laser-tissue welding and laser-tissue soldering, in a rat skin wound-healing model. Various roles for gold nanoparticles are described by Mazzola, L. in 2003 Nature Biotechnology 21(10): 1137-1143, including molecular detection assays, localized payload delivery, tissue ablation triggered by a secondary mechanism such as light activation, and separation.

The gold nanoshell synthesis in Gobin et al. uses basic reduction of tetraethyl orthosilicate, followed by reaction of the silica core nanoparticles with (3-aminopropyl)triethoxysilane (APTES, Sigma-Aldrich, St. Louis, Mo.), and amine groups on the surface of the core allow for deposition of gold colloid. Gold particles (Auroshell™) are commercially available from Nanospectra Biosciences, Inc. (Houston, Tex.), and from Purest Colloids (MesoGold®, Westampton, N.J.). Examples herein use Colloidal Gold Total Protein Stain (BioRad, Hercules, Calif.), however it is envisioned that any commercially available colloidal gold preparation would function similarly in detection of early-stage caries.

Tetracycline in addition to its well-known importance as an antibiotic is a fluorescence incident agent for photometry, for example, for labeling for bone development/formation. Tetracycline however produces tooth discoloration, referred to as “tetracycline teeth” in dentistry. Tetracycline binds to newly formed bone or tooth at the interface and the resultant binding is observed as a line or dot of fluorescence. The phrase, “tetracycline fluorescence” as an agent that binds to newly formed bone or teeth, is capable of fluorescence when illuminated at a pre-determined wavelength of light, and includes, without limitation, all of the members of the tetracycline family as well as additional compositions such as the gold compounds, quantum dot compounds, HiLyte Fluor 750 hydrazide compounds, and any other compounds that share the functional attributes of binding to newly formed bone or teeth and emitting fluorescence or other optical or physical signal upon illumination at a stimulating wavelength.

OsteoSense 750 is a bisphosphonate-conjugated imaging agent used to detect osteogenic activity. OsteoSense 750 acts by targeting hydroxyapatite exposed during times of bone turnover binding thereto with high affinity, thus allowing for in vivo detection and monitoring of skeletal changes. OsteoSense 750 is a commercially available (VisEn Medical, Woburn, Mass.) fluorescent agent that emits light at a wavelength of about 750 nm.

Cy7, or cyanine dye 7, is a fluorescent dye that upon excitation emits light of about 750 nm wavelength. Modified versions of Cy7 are commonly used as fluorescent labels for proteins and antibodies. Cy7 is commercially available from Amersham Biosciences (Piscataway, N.J.). CardioGreen (ICG) is a tricarbocyanine dye that upon excitation emits light of about 800 nm. CardioGreen (ICG) is commercially available from Sigma-Aldrich (St. Louis, Mo.).

IR820 (ICG) is a near-infrared indocyanine dye that upon excitation emits light of about 845 nm. IR820 (ICG) is commercially available from Sigma-Aldrich (St. Louis, Mo.). Far-Green Two is a commercially available dye that that upon excitation, emits light of about in the NIR range. AngioSense 750 is a fluorescence agent that remains localized in vasculature for extended periods of time (approximate half life in plasma 6 h), a property that facilitates imaging, for example, to monitor angiogenesis. AngioSense 750 is commercially available (VisEn Medical, Woburn, Mass.) and emits light at a wavelength of about 780 nm. Genhance 750 is a fluorescence agent, used in the vascular system to facilitate imaging and to monitor angiogenesis. Genhance 750 is a commercially available (VisEn Medical, Woburn, Mass.) fluorescent agent that emits light at a wavelength of about 780 nm. AngioSpark 750 is a fluorescent dye that emits light at a wavelength of about 750 nm. The dye is a macromolecule that is usually useful as it remains localized in vasculature for extended periods of time (approximate half life in plasma 6 hours). AngioSpark 750 has been used to facilitate imaging to monitor angiogenesis, and is commercially available (VisEn Medical, Woburn, Mass.).

Alexa Fluor 750 is a fluorescent dye that upon excitation emits light of about 750 nm wavelength. Alexa Fluor 750 is commercially available from Invitrogen Corp. (Carlsbad, Calif.). Indocyanine Green (ICG) is a tricarbocyanine dye that upon excitation, emits light at wavelengths of about 800 nm, about 820 nm, about 840 nm or at about 860 nm. ICG is commercially available (from H. W. Sands Corp., Jupiter, Fla.) and has been used in infrared photography, the preparation of Wratten filters, and as a diagnostic aid for blood volume determination, cardiac output, or hepatic function. The properties of ICG are described in Landsman et al. 1976 J. Appl. Physiol., 40: 575-583.

Doxorubicin (also known as adriamycin or hydroxyldaunorubicin) is a DNA-interacting cancer drug widely used in chemotherapy. A chemotherapeutic dose of Doxorubicin is in a range of about 60 mg/m² to 75 mg/m². Doxorubicin is fluorescent and emits light at wavelengths of about 550 nm, 600 nm, or 650 nm and this property has been used in cell biology research for measurement of drug efflux pump activities and intracellular localization of various multi-drug resistance proteins, at much lower concentrations than the chemotherapeutic dose. Doxorubicin is commercially available from Sigma-Aldrich (St. Louis, Mo.).

Riboflavin (vitamin B₂) is an easily absorbed micronutrient with a role in a wide variety of cellular processes, for example, energy metabolism. Riboflavin is an easily absorbed, water-soluble micronutrient that supports energy production by aiding in the metabolism of fats, carbohydrates, and proteins. Riboflavin is also needed for red blood cell formation and respiration, antibody production, and for regulating human growth and reproduction. Riboflavin functions as an antioxidant by scavenging damaging particles in the body known as free radicals, and is important for healthy skin, nails, hair growth and general good health, including regulating thyroid activity. As Riboflavin is water soluble, an excess is not stored and is excreted generally in the urine. As a result, Riboflavin has no known toxic dose. The minimum daily recommended dose ranges from 1 mg to 2 mg as a dietary supplement, while a typical therapeutic daily dose ranges from 50 mg to 100 mg. Substantially less Riboflavin is needed in the methods herein for contacting a surface of a tooth. Riboflavin is commercially available from Sigma-Aldrich (St. Louis, Mo.) and is fluorescent, emitting light at a wavelength of, for example, about 450 nm, about 550 nm, about 650 nm, or about 750 nm. The properties of Riboflavin are described in Du et al. 1998 Photochemistry and Photobiology, 68: 141-142.

Chlorophyll A is a green photosynthetic pigment that emits light at a wavelength of, for example, about 600 nm, about 700 nm, or about 800 nm. Chlorophyll A is commercially available from suppliers such as Sigma Chemical (St. Louis, Mo.) and Turner Designs (Sunnyvale, Calif.). As Chlorophyll A is a normal part of a regular human diet, it has no known toxicity.

Bacterial Chlorophylls or bacteriochlorophylls are photosynthetic pigments that occur in various phototrophic bacteria and do not produce oxygen. Bacterial chlorophylls differ structurally from the chlorophylls of higher plants and from each other structurally. Each bacteriochlorophyll absorbs light energy in a different portion of the spectrum. Examples of bacteriochlorophylls are: bacteriochlorophyll a, bacteriochlorophyll b, bacteriochlorophyll c, bacteriochlorophyll d, bacteriochlorophyll e, and bacteriochlorophyll g. Bacteriochlorophyll a and bacteriochlorophyll b are the photosynthetic pigments of purple bacteria. Bacterial Chlorophylls are fluorescent pigments and emit light at a wavelength, for example, about 350 nm, about 450 nm, about 650 nmm or about 750 nm. Bacterial Chlorophylls are available commercially from Sigma-Aldrich (St. Louis, Mo.).

Porphyrin is a heterocyclic macrocycle made from 4 pyrrole subunits linked on opposite sides through 4 methine bridges (═CH—). The extensive conjugated structure of Porphyin makes the compound chromatic, i.e., fluorescent at a wavelength of, for example, about 600 nm, or about 650 nm, or about 700 nm. Porphyrin is commercially available from Sigma-Aldrich (St. Louis, Mo.). Porphyrin is associated with hemoglobin and myoglobin, which are components of an animal based diet, and is therefore a normal part of a regular human diet, thus it also has no known toxicity.

For bioluminescent probes, excitation energy is supplied by a chemical reaction rather than from an incoming source of light. Luciferin and luciferase are an example of a substrate and its associated enzyme, which catalyzes a light-producing reaction, i.e. bioluminescence, and adenosine triphosphate (ATP) is involved in this reaction. Light is emitted (for example at about 500 nm, at about 550 nm, or at about 650 nm) when luciferase is exposed to the appropriate luciferin substrate in the presence of ATP, and photon emission is detected by a light sensitive apparatus such as the optical devices described herein. Luciferase and luciferin have been widely used, for example, to observe biological processes and stages of infection, and are commercially available from Sigma-Aldrich (St. Louis, Mo.).

Further examples of bioluminescent compositions are green fluorescent protein (GFP) and aequorin. These are bioluminescent compositions are isolated from the jellyfish Aequorea victoria. When a calcium ion binds to aequorin, the complex breaks down into apoaequorin and a luminescent composition, which emits blue light (at about 466 nm). Synthetic aequorin is commercially available from Sealite Sciences (Bogart, Ga.) as AQUALITE®. GFP emits light in the lower green portion of the visible spectrum (at about 490 nm to about 570 nm). Synthetic GFP is commercially available from Clontech (Mountain View, Calif.).

The composition known as “quantum dot” consists of a solution of nanometer-scale (roughly protein-sized) atom clusters, exemplified by Qdot® available commercially from Invitrogen (Carlsbad, Calif.). The clusters contain combinations of materials, such as a combination of alkali metals (Li, Na, K, Rd, Cs and Fr), alkaline earth metals (Be, Mg, Ca, Sr, Ba and Ra), transition metals (Sc, Ti, V, Cr, Mn, Fe, Co, Ni, Cu, Zn, Y, Zr, Nb, Mo, Tc, Ru, Rh, Pd, Ag, Cd, Hf, Ta, W, Re, Os, Ir, Pt, Au and Hg), lanthanides (La, Ce, Pr, Nd, Pm, Sm, Eu, Gd, Tb, Dy, Ho, Er, Tm, Yb and Lu) and actinoids (Ac, Th, Pa, U, Np, Pu, Am, Cm, Bk, Cf, Es, Fm, Md, No and Lr) on a silica- or silicone-based core.

Quantum dot preparations have been developed for use as fluors by binding to samples followed by illuminating with light in the UV spectrum. The quantum dot preparations exhibit large molar extinction coefficients, high photostability and strong and size-dependent tunable emission. The tunable aspect of the emission peak is adjusted to be infrared, far-red and red light that accordingly binds to dental enamel by virtue of the commercially available preparations having different combinations of metals and different sizes of particles. In addition, dot particle preparations are available with an overall negative charge or a positive charge, depending on the combination of metals.

Examples herein show that a quantum dot preparation selectively binds to decalcified enamel but not healthy electronically neutral enamel, due to the electronic charge. Furthermore, a quantum dot preparation is conjugated to any of the small compounds described herein, such as tetracycline, calcein, a fluorescent stain used to label intact and living cells (Invitrogen), and their derivatives that have an affinity to decalcified enamel. These conjugates form an “enamel affinity quantum dot” preparation. Decalcified enamel is then detected using the enamel affinity quantum dot as enamel-translucent fluorescence. Alternatively, quantum dots that bind to decalcified enamel are detected by dichroism, such as fluorescence detected circular dichroism.

Yet another example of a useful composition is HiLyte Fluor 750 hydrazide, which is a commercially available fluorescence dye that is used as a detection agent for colloidal gold (AnaSpec, Inc., San Jose, Calif.). HiLyte Fluor 750 hydrazide is a carbonyl-reactive fluorescent labeling dye. It is used for labeling glycoprotein such as horseradish peroxidase (HRP). HiLyte Fluor 750 hydrazide is the longest wavelength carbonyl-reactive HiLyte Fluor dye currently available. Its fluorescence emission is at about 782 nm, well separated from commonly used far-red fluorophores such as HiLyte Fluor 647, HiLyte 680 or allophycocyanin (APC), facilitating multicolor analysis.

The probes herein are suitable for contacting or applying to a mammalian tooth (humans and high value animals) for the detection of an early stage caries lesion to which the probes herein bind, including an amount of a probe of the present methods or a pharmaceutically acceptable salt thereof, which is effective for this detection. The probes according to the methods are those for oral contact or application to a mammalian tooth (humans and high value mammals) that include an effective dose of the probe, alone or together with a significant amount of a pharmaceutically acceptable carrier or buffer. As used herein, the term “pharmaceutically acceptable carrier or buffer” includes any and all solvents, diluents, or other agents, compositions or fluids suited to the effective use of the probe.

The amount of probe used by the dentist is chosen by the individual dentist in view of the patient to be treated. Dosage and administration are adjusted to provide sufficient levels of probe to detect caries. Additional factors which are taken into account include the severity of the caries disease state, e.g., extent of the condition, history of the condition. For example, the desired dosage of the probe depends on the species of the mammal, the body weight, the age and the individual condition, individual pharmacokinetic data, and the mode of administration.

A concentration of a solution of the probe of the present methods or a pharmaceutically acceptable salt thereof to be contacted to a tooth surface, for example an adult human tooth surface, is for example, from approximately 1 ng/ml to approximately 100 ng/ml, from approximately 100 ng/ml to approximately 500 ng/ml, from approximately 500 ng/ml to about 1 μg/ml, from approximately 1 μg/ml to approximately 50 μg/ml, and from approximately 50 μg/ml to approximately 500 μg/ml. Predictive dental doses for children are potentially significantly lower, for example one-half to one-tenth of a concentration suitable for an adult.

A longstanding need in dental medicine is a method for detecting an early stage caries lesion. Compositions herein have not previously been considered as probes for detection of a carious lesion, for example, at a stage associated with initial events such as early demineralization. Another longstanding need is detection of an interproximal lesion, i.e., a lesion located on a surface between teeth. Detecting interproximal lesions at an early stage, i.e., located within one-half of the depth of enamel, using for example bite wing X-rays or by visual clinical inspection, has not previously been possible.

An optical device suitable for detecting the caries with bound probes using to the methods herein includes optical components similar to those found in endoscopes. These optical components include either a rigid or flexible tube containing one or more optical fiber systems, the tube having a channel for mechanical devices, such as a light delivery system used, for example, to illuminate an object under inspection, in the case herein, a surface of a tooth. In certain embodiments, the optical device further includes a device that emits electromagnetic wavelength radiation. Such a device is described in Bukosky et al. (U.S. Pat. No. 6,076,948, issued Jun. 20, 2000).

In one embodiment, the light delivery system includes a light source located outside the oral cavity, with the light directed onto the tooth via an optical fiber system. Alternatively, the optical device contains a built-in light source, such as an LED. In certain embodiments, the optical device includes a lens system to transmit images to the user. The user is able to control the wavelength of the transmitted source, for example, by transmitting light suitable for excitation of a fluorescent probe, a composition that binds to the caries. An example of a hand-held intra-oral light wave detection device is the SharpVision ZE-411 oral endoscope (Sharp Vision Co. Ltd., Guangdong, China).

In certain embodiments, the optical device is an ultra-violet (UV) lamp. UV refers to electromagnetic radiation with wavelengths in the range of about 10 nm to about 400 nm. The UV wavelengths from about 345 nm to about 400 nm produce a “blacklight” effect, i.e., this range of wavelengths causes certain compositions to fluoresce. UV lamps are commercially available from Unilam Co, LTD. (South Korea).

In other embodiments, the optical device is a spectrophotometer. As used here, a spectrophotometer refers to a device for measuring light intensity, i.e., the device measures intensity as a function of the color, or more specifically, the wavelength of light. In certain embodiments, the spectrophotometer is used to detect the fluorescent probe bound to an early caries lesion in a tooth. Spectrophotometers are commercially available from Hitachi Ltd. (San Jose, Calif.). In a related embodiment, the spectrophotometer is a hand-held spectrophotometer such that a technician measures electromagnetic light waves intra-orally (emission or absorbance) from the probes bound to the early stage caries on a tooth. Hand-held spectrophotometers are commercial available from Konica Minolta (Chiyoda-ku, Tokyo).

A probe in embodiments described herein fluoresces at a wavelength within the spectrum of visible light, and is thus detected using a camera to photograph the tooth having an early stage caries lesion to which the probe is bound. As used herein, a camera refers to a device used to capture images as still photographs or as sequences of moving images, for example a charge coupled device (CCD) camera that converts optical brightness into electrical amplitude signals using a plurality of charge coupled devices, and then reproduce the image of a subject using the electric signals without time restriction. A CCD camera is commercially available, for example, from Texas Instruments (Dallas, Tex.).

An optical fiber such as for use in fiber optics is within the scope of the optical devices herein. The optical fiber includes a glass or plastic fiber that transmits light along its length by total internal reflection. The fiber includes a core surrounded by a cladding layer, in which one or more layers of material of lower refractive index are in contact with a core material of higher refractive index. Optical fibers are used herein as light guides, to illuminate an area or locus of a dental surface, including an interproximal surface between teeth. Optical fibers include a coherent bundle of fibers, often along with lenses.

Fiber optics are used by dentists, for example, in preparing a dental filling by light-polymerization of a composition. The hardening process uses blue light with a wavelength of approximately 450 nm produced from a hand-held light source via a fiber rod or fiber taper to the tooth being treated (SCHOTT North America Inc., Southbridge, Mass.). These devices are readily adaptable to the kits and methods herein. Optical devices are previously described as “probes” in prior publications, however as used herein that term means a composition that binds a caries. In general the optical detection device captures at least one or at least two images of the tooth, an image that shows the anatomy of the tooth (enamel, dentin, and enamel-dentin junction) and an image that shows fluorescence of the tooth having a caries lesion and probe bound to the lesion. The image of the anatomy of the tooth is detected using light illumination with a device such as a CCD camera, an optical device, or a spectrophotometer to obtain an image of a caries. This image is superimposed with a fluorescence image of using the superposition of the two images. A clinician accurately determines the size, depth and location of the caries. Software to superimpose images is within the scope of the methods and kits herein.

Using the kits and methods herein a tooth surface is contacted with one or more probe compositions. For example, a thin lamella or matrix is coated with probe, and the matrix is applied to the tooth. The matrix is a thin strip of paper, plastic tape, or another convenient applicator. Alternatively an injection type of syringe having a barrel containing a solution of the probe is used to apply a small volume of the probe as a soak. The interproximal region is made more accessible by pre-treating the area with a wedge to widen the space between two or more teeth.

After probe has been contacted to a tooth or teeth and prior to visualizing with an optical device, the area is rinsed at least once with water and/or an aqueous solution to remove excess probe. After detecting the probe, a device, for example a syringe, is used to apply a fluid to further remove probe from the tooth or teeth. The fluid used to remove the probe is for example hydrogen peroxide, for example Opalescence Boost Teeth Whitening system sold by Ultradent Inc. (South Jordan, Utah) contains 38% hydrogen peroxide. Alternatively, the fluid used to remove the probe is a solution of phosphoric acid, sodium phosphate monobasic, sodium phosphate dibasic, methylene phosphoric acid, sodium chloride, potassium chloride, pyrophosphate dibasic, and pyrophosphate tetrabasic. For example, the fluid used to remove the probe is a one of a phosphoric acid solution at a concentration of 5%, 10%, 20%, 40% by weight, or has a range of concentration of 5%-10%, 5%-20%, 10%-20%, 5%-40%, 10%-40% or 20%-40% phosphoric acid solution by weight, or other solutions herein.

As described in this application, fluid for removing probe from the tooth is generally aqueous, or is an organic solvent and in various embodiments includes one or more buffers to provide a stable pH in presence of acid or alkali or upon dilution, and further includes a cation or ion, for example calcium or chloride. The fluid for removing the probe is applied during a time period, for example 1-15 minutes, to interact with the probe on the tooth. Removing the probe using a fluid rinse follows binding and prior to detecting a pre-carious lesion in one embodiment. Alternatively, the probe bound to the lesion is detected directly.

An example of a dental use of optical fibers includes lighting of handpieces. Dental instruments typically include a light source to illuminate the treatment area. A light guide is built into the instrument handpiece for this purpose (SCHOTT North America Inc., Southbridge, Mass.), and such instruments are adapted to the purposes herein.

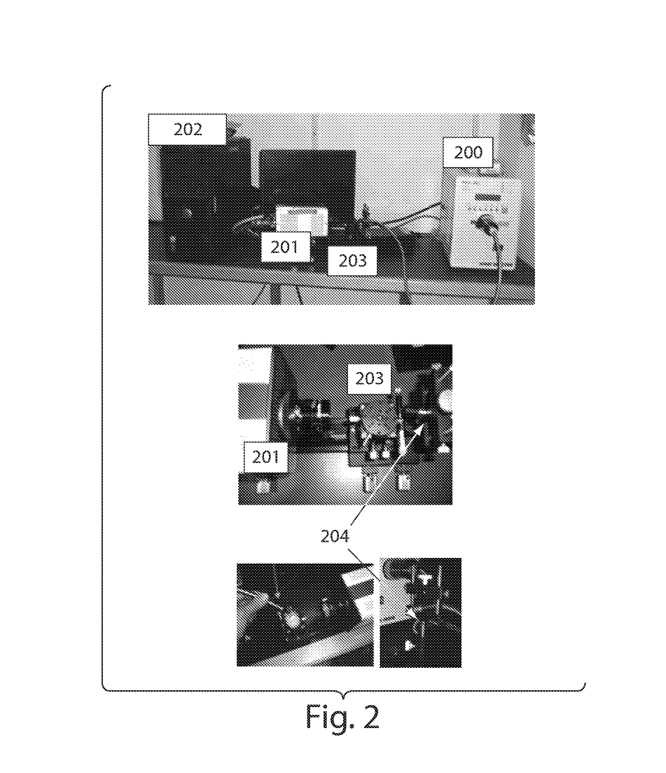

FIG. 2 shows an optical device system for detecting a probe that is bound to an early stage caries lesion in an experimental tooth or portion thereof. A tooth is placed in stage 203. A light source 200, a MAX301 xenon light (Asahi Spector, China), illuminates an area of the tooth at a wavelength that allows for detection of binding of the probe, i.e., illuminates with light having a wavelength of absorption or emission. In certain embodiments, an optical fiber 204 (Olympus, Melville N.Y.) transmits and focuses the electromagnetic light waves emitted by light source 200 and direct the waves at an area on the tooth that is located on stage 203. The optical fiber 204 is modified to direct the electromagnetic light waves to form a spot illumination, using a silicon cap and paper wrapping. A camera 201, a MC285SPD-L0B0 camera (Texas Instruments, Dallas, Tex.), captures the image of the detectable probe and transmits it to computer 202, Optiplex 20 1L (Dell Inc., Round Rock, Tex.). A computer 202 uses Capture eBase (Solution Systems, Inc., Rolling Meadows, Ill.), a software program for controlling light source 200, for example, the software controls the wavelength of light source 200, for example, to transmit light suitable for excitation of the probe.

Dental caries remain a global chronic disease (Brown L. J., et al. 2000 J Am Dent Assoc 131(2): 223-31). Traditional visual-tactile methods and radiographic methods are generally used to detect caries that are more advanced, for example, advanced caries that are found at a depth of greater than 500 nm of the enamel. These traditional methods fail to diagnose many incipient carious lesions (Stookey G. K., 2005 Dent Clin North Am, 49(4): 753-770). A location such as interproximal prevents these caries from being detected by methods other than by bitewing radiography. However, bitewing radiographs exhibit low accuracy and sensitivity values for detecting interproximal lesions, 65% and 45% respectively (Kang B. C., et al. 1996 Caries Res. 30(2): 156-62; White S. C., et al. 1997 Dentomaxillofac Radiol. January; 26(1): 32-8). Tooth decalcification, about 40% to about 60%, is necessary before lesions are visible in a radiographic image. Thus, incipient lesions are not visible on radiographic images. Further, radiographs have generally been found to underestimate the actual size and/or depth of a carious lesion (Wenzel A., et al. 1990 Caries Res 24(5): 327-33; White S. C., et al. 2003 Oral Radiology: Principles and Interpretation. 5th edition Mosby). By the time an interproximal lesion is visible on a bitewing radiograph invasive class II restorations are required, subjecting the patient's tooth to invasive dental treatments and procedures.

Detection of early caries which are in a reversible state reduces the probability that a subject will need an invasive dental restoration and treatment for caries that generally appear subsequent to these restorations. (Marinho V. C., et al. 2003 Cochrane Database Syst Rev, 1: CD002278). There is a need for an early stage dental caries as an optimal diagnostic tool. Such a system must have good diagnostic performance, be noninvasive, easy to use, and reasonably inexpensive (Kuhnisch J., et al. 2006 Caries Res., 40: 104-111).

Optical tools/devices for caries detection include digital imaging with transillumination (DIFOTI; Schneiderman A., et al. 1997 Caries Res, 31: 103-110), quantitative light-induced fluorescence (QLFI and QLF II; Heinrich-Weltzien R., et al. 2003 Quintessence Int, 34(3): 181-188), and laser fluorescence (DIAGNOdent; Shi X., et al. 2000 Caries Res, 34: 151-158). Optical diagnostic tools that measure changes in light scattering have potential for detecting caries. However, drawbacks affecting suitability include, for example, DIFOTI only operating in the visible light wavelength range, and penetrating insufficiently deeply through enamel. QLF is costly and generally only purchased by universities and research centers (Heinrich-Weltzien R., et al. 2003 Quintessence Int, 34(3): 181-188). DIAGNOdent (Kayo, Biberach/Riss, Germany) detects the Near Infrared (NIR) fluorescence from bacterial porphyrins, however detects only advanced caries that have accumulated a large amount of bacterial byproducts. A study assessing enamel lesions using DIAGNOdent found the combination of the method and device had a sensitivity of only 40% (Shi X., et al. 2000 Caries Res, 34: 151-158). Thus, a device that reliably detects caries, particular interproximal caries, is needed, to provide consistent and reliable images that accurately represent the size and/or depth of a carious lesion, and promotes an active preventive therapy, for example a system that allows a clinician to make an early diagnosis and to formulate a treatment plan tailored to the individual (Nyvad B., 2004 Caries Res., 38: 192-198).

Wavelengths of light studied for scattering characteristics include scattering probability that decreases with increasing wavelength (Hall A., et al. 2004 J Dent Res, 83 (Spec Iss C): C89-C94). A long wavelength of light, 1310 nm, was used to image lesions, discriminate demineralization, staining, and pigmentation, and to investigate developmental defects such as fluorosis. However, this method was not tested in vivo, and an optical device to detect a wavelength of 1310 nm would be extremely expensive.

Early detection of caries permits opportunities for preventive dental measures including minimizing caries from recurring near a restoration, reducing incidence of early restoration failure, decreasing incidence of tooth fracture related to weakened cusps resulting from large restorations, and retaining vitality of dental pulp throughout the lifetime of the subject (Dennison J. B., et al. 2005 Dent Clin N Am, 49: 525-545).

Sensitivity is an important characteristic for detecting a disease that results in negative consequences such as pain, expense and loss of self-esteem. Sensitive tests are especially helpful at the early stages of a diagnostic workup (Fletcher R. H., 2005 Clinical Epidemiology, Chapter 3: Diagnosis, Lippicott). While dental caries do not generally have lethal consequences, progression of this destructive process becomes irreversible and painful and expensive to treat. A sensitive detection method for detecting caries is needed.

Teeth have a number of components and layers including enamel, dentin, cementum, and dental pulp. Enamel varies in thickness across the surface of the tooth and is often thickest at the cusp, up to 2.5 mm, and thinnest at the border, which is seen clinically as the cementoenamel junction. Individual enamel rods measure about 4 μm to about 8 μm in diameter and are the primary component of enamel. An enamel rod, known also as enamel prism (101, FIG. 1A), contains crystalline hydroxyapatite organized in an oriented pattern. These calcified microscopic rods are perpendicular to and radiate from the dentin. The area between individual enamel rods is about 10 μm wide, and is referred to as interrod enamel. Bacteria and acid enter the interrod enamel and result in a white spot (100, FIG. 1A) caries lesion or an incipient caries to form over time.

Examples herein use probes (e.g., fluorescent and bioluminescent) and show that these probes bind to a caries lesion and are detectable. Examples herein show, for example, contacting a fluorescent probe to dental caries, illuminating the tooth with light of an excitation wavelength, and detecting a light signal having at least one emission wavelength. Multiple methods are provided for illuminating the tooth, for example, illuminating the interproximal area directly or illuminating the area through the occlusal layer. Data show that the methods provided herein detected more than 90% of the actual size and/or depth of caries, compared to bitewing X-rays.

It was observed in examples herein that the methods, occlusal illumination and interproximal illumination, resulted in improved sensitivity, specificity, positive predictive value, and negative predictive value results compared to X-rays. The “positive predictive value” is the probability of a true disease state following or in the presence of a positive assessment. The “negative predictive value” is the probability of a false disease state following or in the presence of a false assessment. Examples herein show that the mean negative predictive value of X-rays was about 35%, indicating that the true caries are expected to be found in 65% of negative assessments for this method, i.e., much too high a false negative predictive value. The negative predictive values of occlusal illumination and interproximal illumination observed herein were much greater compared to the negative predictive values obtained for X-ray data. An increased negative predictive value indicates to a dentist that a negative assessment truly indicates the absence of a lesion.

Data obtained in examples herein thus show higher user reliability compared to X-rays. Assessments using each of occlusal illumination images and interproximal illumination images resulted in improved sensitivity compared to X-ray images. Examples herein also showed that assessments using interproximal illumination images resulted in increased sensitivity in detecting early stage caries compared to assessments using occlusal illumination images. It is likely that illuminating through the tooth structure/geometry reduces the amount of light reaching a fluorescent probe. Without being limited by any particular theory or mechanism of action, it is envisioned that other variables affect the observed sensitivity and specificity of the methods described herein, including duration of illumination, distance from the tooth during the period of illuminating, etc.

Enamel and dentin generally autofluoresce at wavelengths of about 500 nm to about 700 nm. Data herein show that fluorescent probes respond to a longer excitation wavelength and minimize an overlapping fluorescence range to minimize effects of autofluorescence. A longer wavelength of light in the NIR range yields less scatter, resulting in greater amounts of light penetrating teeth more completely without compromising the contrast of the obtained image.

An aspect of the invention provides a method for detecting an early stage dental caries in a subject, the method including: contacting a caries lesion which is at an early stage, by selective binding of an optically detectable probe to the caries, such that the probe is a molecule capable of binding the caries; and, detecting the caries having bound probe using an optical device.

A related embodiment of the method provides detecting the bound probe by contacting a tooth with a fluorescent probe, which is at least one of: a tetracycline, a HiLyte Fluor, an OsteoSense 750, a Cy7, a Qdot, a CardioGreen (ICG), an IR820 (ICG), a Far-Green Two, an AngioSense 750, a Genhance 750, an AngioSpark 750, an Alexa Fluor 750, an Indocyanine Green, a Doxorubicin, a Riboflavin, a Chlorophyll A, a bacterial Chlorophyll and a Porphyrin; and illuminating the tooth at an excitation wavelength, such that diagnosing includes detecting an area of light emission at an emission wavelength. In general, the early stage caries is prior to cavitation or advanced demineralization. In a related embodiment, detecting the caries is observing an area by photometry.

A dentist identifies a demineralized tooth, for example a tooth having an early stage caries or advanced caries using the methods, compositions and kits herein, and then remineralizes the tooth. Conventional methods of remineralizing teeth include for example applying calcium phosphate compositions. Enamelon for example is a commercially available white toothpaste with calcium and a blue toothpaste with phosphate and fluoride (Enamelon Inc.; Yonkers, N.Y.). Squeezing the tube produces side-by-side stripes to produce a remineralizing treatment. Alternatively, dentists apply to teeth one or more of remineralizing gels and/or foams, for example Denti-Foam 1.23% APF Fluoride Foam (Medicom Inc., Tonawanda, N.Y.) or a topical gel containing 100-150 ppm fluoride. Dentists administer doses of these products that are well below a level of toxicity, and instruct their patients not swallow the gel or foam.

Data in examples herein show that teeth and tooth surfaces contacted with bisphosphonate compositions or pyrophosphonate compositions showed reduced or inhibited caries progression or loss of hydroxyapatite. Exemplary bisphosphonate compositions and pyrophosphonate compositions used herein include or were attached to a fluorescent dye. The bisphosphonate composition or pyrophosphonate composition are envisioned to include compounds that are chemically modified by substitutions or deletions of at least one functional group, such that the resulting compositions bind more effectively to teeth or other skeletal bone that contain hydroxyapatite. Thus, the teeth and skeletal bones were protected from further loss of tooth material or bone material, as the loss was inhibited. Further, the tooth or tooth material so treated was found to contain less porous hydroxyapatite, which is better able to be remineralized when contacted with remineralizing agents, for example compositions including calcium phosphate. Methods and techniques for altering the structure of a molecule are well known in the art of chemistry so that modified bisphosphonates and pyrophosphonates are within the scope of active ingredients included in the compositions herein.

Bisphosphonates are a class of molecules having two phosphonate (PO₃) groups covalently linked to a tetrahedral carbon atom that is further covalently attached to two other functional groups. Bisphosphonate compounds have been orally administered to subjects to prevent osteonecrosis and osteoporosis. Without being limited by any particular mechanism of action, bisphosphonates are efficacious for treating these diseases and conditions by promoting osteoblasts or inhibiting osteoclasts in circulation, in the body, or both.

Oral administration of bisphosphonate compounds involves delivering the compounds into the alimentary canal and digestive tract through which the composition is absorbed into the body. Bisphosphonate compounds for alleviation of osteonecrosis and osteoporosis are shown in Dansereau et al., U.S. Pat. No. 7,645,460 B2 issued Jan. 12, 2010 and in Little et al., U.S. Pat. No. 7,612,050 B2 issued Nov. 3, 2009. Thus, methods for administering these compounds have focused on improving delivery of bisphosphonates to the gastrointestinal tract by improving the coatings and concentrations of molecules compound with the active agent.

Pyrophosphonate compounds are salts or esters of pyrophosphoric acid (H₄P₂O₇) that are used as food additives and in industrial processes. See Hart, U.S. Pat. No. 4,254,073 issued Mar. 3, 1981. Tetra potassium pyrophosphate for example has been used in detergents and dispersants. This class of compounds has also been used as an emulsifying agent and a chelating agent as in water softening. Chelating agents are organic compounds that form bonds with metals through two or more atoms of the organic compound.

Without being limited by any particular theory or mechanism of action, it is here envisioned that compositions containing a bisphosphonate and/or a pyrophosphonate or derivatives thereof when contacted to demineralized teeth or eroded teeth specifically bind to hydroxyapatite and prevent breakdown or loss of hydroxyapatite in areas of porous or demineralized tooth material. These compositions are also capable of strengthening hydroxyapatite and inhibiting caries progression. The hydroxyapatite is found in the porous or demineralized layers of enamel or dentin. The compositions herein containing a bisphosphonate or a pyrophosphonate or both consequently enhance dental remineralization and are exemplary dental remineralization enhancers.

The terms “bisphosphonate” and “diphosphonate” are used interchangeably herein and refer to a molecule or compound which has a molecular or compound structure that contains two phosphate (PO₃⁻) groups covalently linked to a tetrahedral carbon atom. The term bisphosphonate includes an acid, a salt, a hydrate, a polymorph, a solvate, and a derivative thereof. Bisphosphonate binds exposed hydroxyapatite found in teeth and tooth surfaces.

The terms “pyrophosphate” and “pyrophosphonate” are used interchangeably herein and refer a molecule or compound which has a structure that contains a pyrophosphate ion and/or is an anion, a salts, or an esters of pyrophosphoric acid. The term pyrophosphonate includes for example a hydrate, a polymorph, a solvate, and a derivative thereof. Pyrophosphonate binds exposed hydroxyapatite found in teeth and tooth surfaces.

The term “derivative”, as used herein refers to a chemically related form of a molecule or a compound having an additional or modified substituent, for example, a different functional group or atom attached to an atom of the molecule or compound.

The term “dental remineralization enhancer” refers to a composition that binds to teeth or tooth surfaces and alters or modifies the chemical state or surface of the tooth or tooth components such that the hydroxyapatite in the teeth or tooth surfaces is coated or strengthened, for example, exposed hydroxyapatite in teeth or tooth surfaces, and thus these surfaces are less porous.

Without being limited by any particular theory or mechanism of action, it is here envisioned that compositions including a bisphosphonate or a pyrophosphonate or both specifically bind hydroxyapatite in teeth or tooth surfaces having a caries, such that hydroxyapatite is in contact with material that strengthens the hydroxyapatite and prevents further incursion by a caries lesion or bacteria associated with the caries lesion.

Hydroxyapatite is a calcium phosphate material, Ca₁₀(PO4)₆(OH)₂, comprising a major component of the highly mineralized matrix of natural mammalian bone and teeth. Engineered hydroxyapatite is characterized as having a high degree of biocompatibility and bioreactivity with mammalian cells, characteristics which have made the material a useful bone substitute and implant material. Methods of producing and engineering hydroxyapatite materials are well known in the art.

Compositions herein bind to and specifically hydroxyapatite on a tooth, for example a tooth characterized as have porous, missing, or exposed hydroxyapatite material. The composition is without limitation capable of binding to a hydroxyapatite containing layer of a tooth and/or bone. A composition including a bisphosphonates and/or a pyrophosphonate or a portion thereof binds to eroded tooth or a bone material containing hydroxyapatite, such that the tooth or bone includes less porous hydroxyapatite and is capable of more effectively attaining the organized structure characteristic of healthy tooth or bone material when contacted (prior to or subsequent to) with a remineralizing agent.

Examples of compositions that bind to hydroxyapatite and enhance remineralization of teeth include for example, OsteoSense 750, zoledronate, aledronate, pamidronate, risedronate, ibandronate, incadronate, minodronate, olpadronate, neridronate, etidronate or etidoronate, clodronate, tiludronate, and derivatives thereof.

OsteoSense 750 is a bisphosphonate-conjugated imaging agent used to detect osteogenic activity. OsteoSense 750 is commercially available from VisEn Medical, Woburn, Mass. and is a fluorescent agent that emits light at a wavelength of about 750 nm.

Zoledronate, also known as zoledronic acid or Zometa®, is (1-hydroxy-2-imidazol-1-yl-1-phosphono-ethyl)phosphonic acid and is a commercially available bisphosphonate drug for treating hypercalcemia (high blood levels of calcium) in patients with by cancer such as myeloma. The drug reduces or prevents bone fractures and reducing bone pain associated with bone metastases that wear away portions of bone, reducing or preventing osteolytic bone lesions (Novartis Pharmaceuticals Corporation; East Hanover, N.J.).

Aledronate or Fosomax® (sodium [4-amino-1-hydroxy-1-(hydroxy-oxido-phosphoryl)-butyl]phosphonic acid trihydrate) is a commercial bisphosphonate drug for treatment of osteoporosis in men and women associated with hormone changes or disease (Merck; Whitehouse Station, N.J.). The drug is also used to treat other bone diseases including Paget's disease (osteodystrophia deformans) which is a chronic disease of the bones characterized by bowing and deformation of the bones of the spine, pelvis, legs, or skull. The condition is associated with overactive osteoclast resorption followed by disorganized bone production.

Pamidronate (3-amino-1-hydroxypropane-1,1-diyl)bis(phosphonic acid)) is a commercial drug sold as Aredia® by Norvartis Pharmaceuticals Corporation (East Hanover, N.J.) used to treat osteoporosis and bone disorders including Paget's disease.

Risedronate (1-hydroxy-1-phosphono-2-pyridin-3-yl-ethyl)phosphonic acid is a commercially available bisphosphonate drug sold as Actonel® (Sanofi-Aventis; Bridgewater, N.J.) used to treat postmenopausal osteoporosis.

Ibandronate or BONIVA® is used for osteoporosis treatment in women after menopause (Roche; Nutley, N.J.). Incadronate disodium (also called YM175 or cyclopentyldibutylphosphine oxide dibutylphosphorylcyclopentane) is a bisphosphonate for treatment of osteoporosis including preventing bone fractures (Yamanouchi Pharmaceutical Co.; Tokyo, Japan).

Minodronate (YM529; 1-Hydroxy-2-(imidazo(1,2-A)-pyridin-3-yl)ethylidene)bisphosphonic acid monohydrate) is a newly developed nitrogen-containing bisphosphonate that inhibits the bone metastases and has been used to treat or prevent vertebral fractures in patients with osteoporosis (Yamanouchi Pharmaceutical Co.; Tokyo, Japan). Olpadronate (3-(dimethylamino)-1-hydroxypropane-1,1-diyl]bis-phosphonic acid) is a bisphosphonate used to treat bone defects (Gador Pharmaceutical Laboratory; Buenos Aires, Argentina).

Neridronate (6-Amino-1-hydroxyhexane-1,1-diyl)bis-1phosphonic acid) or neridronate sodium hydrate is a bisphosphonate used to treat postmenopausal women for osteoporosis (LGM Pharmaceuticals Inc.; Boca Raton, Fla.).

Etidronate or Didronel (1-hydroxy-1-phosphono-ethyl)phosphonic acid) is a bisphosphonate compound used to treat Paget's disease, and heterotopic ossification (growth of bone tissue in an area of the body other than the skeleton) in total hip replacement surgery or injury to the spinal cord (MGI Pharmaceuticals Inc.; Minneapolis, Minn.).

Clodronate or Bonefas® (dichloro-phosphono-methyl)phosphonic acid is a bisphosphonate compound used to reduce the occurrence of bone metastases in the post-surgical treatment of breast cancer patients (Schering AG; Berlin, Germany).

Tiludronate or Skelid®, ((4-chlorophenyl)thio]methylene}bis(phosphonic acid)) is a commercially available bisphosphonate medication used to treat Paget's disease (Sanofi-Aventis Inc.; Bridgewater, N.J.).

Without being limited by any particular theory or mechanism of action, it is envisioned that the amounts of bisphosphonate administered to detect caries and/or prevent demineralization is non-toxic and would not negatively affect bone structures or cause necrosis). In certain embodiments, an a bisphosphonate compound such as OsteoSense750 solution (20 nmol/150 μl) is diluted to a final concentration of 1.33×10⁻³ nmol/μl, which amounts to 1.46×10⁻⁶ mg/μl. As a 1 μl aliquot of this solution is used for each treatment area for example twelve treatment areas, accordingly a total of twelve μl aliquots of the diluted OsteoSense are used for each treatment. Two caries treatments (e.g., checkups or restorative appointments) are generally performed for each patient per year, thus a total of 24 μl (a total of 3.52×10⁻⁵ mg) of diluted OsteoSense solution are used per patient per year. Several oral bisphosphonate products are on the market for osteoporosis treatment-alendronate (70 mg once weekly), risedronate (50 mg weekly), and ibandronate (150 mg monthly). For example, two Alendronate formulations were approved by the U.S. Federal Drug Administration in October 2000: one 70-mg tablet per week for osteoporosis treatment and one 33-mg tablet per week for osteoporosis prevention. An adult using the preventive formulation would receive 1680 mg alendronate per year. Thus, the twice-yearly topical administration of any of the bisphosphonate-ICG probes described herein for diagnostic dentistry would total only 3.52×10⁻⁵ mg per year, which is only 1/4.7×10⁶ of the oral bisphosphonate dose currently being used for osteoporosis treatment in one year. Thus, the caries detection and remineralization methods described herein are well-within the amount allowed for other treatments and because of the small amount should not have any of those treatments associated negative side effects.

### Pharmaceutical Compositions

The present invention provides pharmaceutical compositions for enhancing remineralization of a tooth of a subject and/or inhibiting or preventing caries progression or loss of hydroxyapatite. In certain embodiments, the pharmaceutical compositions are compounded as a formulation for administering to a subject's tooth or oral cavity. In related embodiments, the compositions may be compounded for sustained release of the bisphosphonate and/or the pyrophosphonate locally, or otherwise formulated to provide effective treatment of a tooth or bones in or adjacent to the oral cavity.

The pharmaceutical composition is compounded as a pure and non-toxic formulation for administering to a human subject, particularly to a tooth or to the oral cavity of a subject. In certain embodiments, these compositions optionally further include one or more additional agents, for example therapeutic agents.

The additional agent is a compound, composition, biological or the like that initiates, enhances, prolongs or stabilizes the ability of the bisphosphonate and/or the pyrophosphonate to remineralize the tooth or bone. Also included are therapeutic agents that may beneficially or conveniently be provided at the same time as the bisphosphonate and/or the pyrophosphonate, such as agents used to treat the same, a concurrent or a related symptom, condition or disease. The drug may include without limitation anti-tumor, antiviral, antibacterial, anti-mycobacterial, anti-fungal, anti-proliferative or anti-apoptotic agents, as are well known in the art. See for example, Goodman & Gilman's The Pharmacological Basis of Therapeutics, 9th Ed., Hardman, et al., eds., McGraw-Hill, 1996, the contents of which are herein incorporated by reference herein.

As used herein, the term “pharmaceutically acceptable carrier” includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences Ed. by Gennaro, Mack Publishing, Easton, Pa., 1995 provides various carriers used in formulating pharmaceutical compositions and known techniques for the preparation thereof. Some examples of materials which can serve as pharmaceutically acceptable carriers include, but are not limited to, sugars such as glucose and sucrose; excipients such as cocoa butter and suppository waxes; oils such as peanut oil, cottonseed oil, safflower oil, sesame oil, olive oil, corn oil, and soybean oil; glycols such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, preservatives and antioxidants can also be present in the composition, according to the judgment of the formulator.

### Therapeutically Effective Dose

Treatment of demineralized or carious teeth and bone by methods provided herein involves contacting a tooth or the oral cavity with a pharmaceutical composition, for example, administering a therapeutically effective amount of a pharmaceutical composition containing a bisphosphonate and/or a pyrophosphonate, to a subject in need thereof, in such amounts and for such time as is necessary to achieve the desired result.

The compositions according to the method of the present invention may be administered using any amount and any route of administration effective for remineralizing and/or inhibiting loss of bone material in teeth. Thus, the expression “amount effective for enhancing remineralization of a tooth of a subject and/or inhibiting or preventing caries progression or loss of hydroxyapatite”, as used herein, refers to an amount of composition applied locally, sufficient to beneficially treat or prevent the disease or condition in teeth and/or bones.

The exact dosage is chosen by the individual dentist in view of the patient to be treated. Dosage and administration are adjusted to provide sufficient levels of the active agent(s) or to maintain the desired effect. Additional factors which may be taken into account include the severity of the disease state, e.g., intermediate or advanced levels of demineralization; diet, time and frequency of administration; route of administration; drug combinations; reaction sensitivities; and tolerance/response to treatment. Long acting pharmaceutical compositions might be administered hourly, twice hourly, every 3 to four hours, daily, twice daily, every 3 to 4 days, every week, or once every two weeks depending on for example the rate of disappearance of the composition by fluids found in and introduced to the tooth or into the oral cavity.

The active agents of the invention are preferably formulated in dosage unit form for ease of administration and uniformity of dosage. The expression “dosage unit form” as used herein refers to a physically discrete unit of active agent appropriate for the patient and/or subject to be treated. It will he understood, however, that the total daily usage of the compositions of the present invention will be decided by the attending dentist within the scope of sound medical judgment. For any active agent, the therapeutically effective dose can be estimated initially either in in vitro assays using isolated teeth or in ex vivo or animal models, as provided herein, such as mice, rats, rabbits, dogs, or pigs. The animal model herein is used to determine a desirable concentration and total dosing range and route of administration. Such information is then used to determine useful doses and routes for administration in humans.

A therapeutically effective dose refers to that amount of active agent that ameliorates the symptoms or condition or prevents the loss of hydroxyapatite in teeth or bones. Therapeutic efficacy and toxicity of active agents can be determined by standard pharmaceutical procedures in cell cultures or experimental animals, e.g., ED50 (the dose is therapeutically effective in 50% of the population) and LD50 (the dose is lethal to 50% of the population). The dose ratio of toxic to therapeutic effects is the therapeutic index, and it can be expressed as the ratio, LD50/ED50. Pharmaceutical compositions that exhibit large therapeutic indices are preferred. The data obtained from cell culture assays and animal studies are used in formulating a range of dosage for human use.

The daily dosage of the products may be varied over a wide range, such as from 0.001 to 100 mg per adult human per day. For dental administration, the compositions are preferably provided in the form of a solution, for example the composition is diluted, containing 0.001, 0.01, 0.05, 0.1, 0.5, 1.0, 5.0, 25.0, 50.0, 100.0, or 500.0 micrograms of the active ingredient for the symptomatic adjustment of the dosage to the patient to be treated.

A unit dose typically contains from about 0.001 microgram to about 500 micrograms of the active ingredient, for example from about 0.1 micrograms to about 100 micrograms of active ingredient, for example from about 1.0 micrograms to about 10 micrograms of active ingredient. An effective amount of the drug is ordinarily supplied at a dosage level of from about 0.0001 mg/kg to about 25 mg/kg of body weight per day. For example, the range is from about 0.001 to 10 mg/kg of body weight per day, or from about 0.001 mg/kg to 1 mg/kg of body weight per day. The compositions may be administered on a regimen of, for example, one to four or more times per day. A unit dose may be divided for example, administered in two or more divided doses.

### Administration of Pharmaceutical Compositions

As formulated with an appropriate pharmaceutically acceptable carrier in a desired dosage, the pharmaceutical composition provided herein is administered to humans and other mammals by methods of administration including topically and bucally. For example the pharmaceutical composition is administered to the tooth or oral cavity as a solution, ointment, or drop.

Topical administration includes contacting the pharmaceutical composition to the tooth or to an adjacent tooth, or to the oral cavity, for example by injection into the gingiva, palate, tongue, or mucosal surface of cheeks.

Liquid dosage forms for topical, buccal or other administration include, without limitation, pharmaceutically acceptable emulsions, microemulsions, solutions, suspensions, syrups and elixirs. In addition to the active agent(s), the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the compositions may include wetting agents, and emulsifying and suspending agents.

Dosage forms for topical administration of an inventive pharmaceutical composition include ointments, films, pastes, creams, lotions, gels, powders, solutions, sprays, inhalants, or patches. The active agent is admixed under sterile conditions with a pharmaceutically acceptable carrier and any needed preservatives or buffers as may be required. For example, topical routes or buccal routes of administration are achieved with aqueous drops, a mist, an emulsion, or a cream. Administration may be therapeutic or it may be prophylactic. The invention includes topical devices or products which contain disclosed compositions (e.g., gauze bandages or strips), and methods of making or using such devices or products. These devices may be coated with, impregnated with, bonded to or otherwise treated with a composition as described herein.

Topical administration of compositions including a bisphosphonate and/or a pyrophosphonate are for example performed by using a patch, film, membrane, sponge, or other device which provides controlled direct delivery of the compositions to the tooth or oral cavity. Dosage devices can be made by dissolving or dispensing the compound in the proper medium, and attaching the devices to the tooth or area in the oral cavity using for example adhesives and/or resins. Absorption enhancers can also be used to increase the flux and release of the compound from the device. The rate can be controlled by either providing a rate controlling material for example a polymer or by dispersing the compound in a matrix or gel.

Injectable preparations are sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use, or can be autoclaved or irradiated or sterilized by methods known in the art.

To prolong the effect of an active agent, it is often desirable to slow the absorption of the agent from subcutaneous or intramuscular injection. Delayed absorption of a parenterally administered active agent may be accomplished by dissolving or suspending the agent in an oil vehicle. Injectable depot forms may be made by forming microencapsule matrices of the agent in biodegradable polymers such as polylactide-polyglycolide. Depending upon the ratio of active agent to polymer and the nature of the particular polymer employed, the rate of active agent release can be controlled. Examples of other biodegradable polymers include poly(orthoesters) and poly(anhydrides). Depot injectable formulations are also prepared by entrapping the agent in liposomes or microemulsions which are compatible with body tissues.

Solid dosage forms for topical administration and/or buccal administration include capsules, tablets, pills, powders, and granules. In such solid dosage forms, the active agent is mixed with at least one inert, pharmaceutically acceptable excipient or carrier such as sodium citrate or dicalcium phosphate and/or a) fillers or extenders such as starches, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof.

Continuation-in-part utility application Ser. No. 12/729,060 filed Mar. 22, 2010 in the United States Patent and Trademark Office, which claims the benefit of provisional application Ser. No. 61/161,978 filed Mar. 20, 2009 and utility application Ser. No. 11/787,158 filed Apr. 13, 2007, which claims the benefit of provisional applications having Ser. Nos. 60/792,768 filed Apr. 18, 2006, 60/819,135 filed Jul. 7, 2006, 60/850,922 filed Oct. 11, 2006, and 60/901,421 filed Feb. 15, 2007 in the U.S. Patent and Trademark Office, are hereby incorporated by reference herein in their entireties.

The invention now having been fully described is exemplified by the following examples and claims, which are exemplary only and are not intended to be construed as further limiting. The contents of all references cited are hereby incorporated herein by reference.

### EXAMPLES

### Example 1

### Detection of Probe Fluorescence in Extracted Teeth Correlated with Presence of Caries

To assess detection of caries using fluorescent probes, enamel samples from extracted teeth were analyzed to identify and obtain those samples having at least one “white spot” (100, FIG. 1A) caries lesion. White spots were divided into two stages: visible white spots, and dull white spots on the enamel surface. Teeth were illuminated and examined for evidence of autofluorescence. No autofluorescence was observed in the teeth after illuminating them with ultra-violet light, therefore the possibility of using fluorescent agents to detect white spots was facilitated by this observation.

The enamel samples from extracted teeth were incubated in a tetracycline solution (1 mg/ml) for 60 seconds, and then were washed with phosphate buffered saline (PBS). The samples were tested using ultra-violet wavelength light of 260 nm (Ultra Violet lamp SB-4W). Fluorescence was determined using a hand-held device attached to an Olympus spectrophotometer. These teeth were observed to have a clear locus of bright yellow fluorescence. The locus of fluorescence was located in the area corresponding to the white spot identified by histological examination, indicating the tetracycline was bound to the white spot caries.

### Example 2

### Histological Analysis

Teeth having white spots were analyzed laterally by electron microscopy, and following obtaining transverse sections, regions of fluorescence were correlated with white spots caries. The teeth were also analyzed histologically to determine the absence or presence of early stage caries. The results were compared to areas observed to have fluorescence from the fluorescent probes.

### Example 3

### Detection of Caries in Bovine Teeth by Colloidal Gold Probes

To determine the capabilities of colloidal gold probes to detect caries, extracted bovine teeth were prepared using a model test system that includes etching the teeth with acid.

Bovine teeth were cleaned by soaking for 10 to 30 seconds in 10% hydrochloric acid. A model test system for caries was established by etching select surfaces with dental etching gel, non-silica 10% phosphoric acid etch gel, for 30 seconds. Control samples were retained in absence of etching.

Colloidal Gold Total Protein Stain (BioRad, Hercules, Calif.) was applied to each of the test samples and to the negative control samples, by soaking for 20-30 seconds. Samples were then rinsed with distilled water. Samples were further stained with Silver Stain Plus kit (Bio-Rad), derived from a method developed by Gottleib and Chavko (1997 Anal Biochem 165: 33). All tooth samples were then illuminated with near-infrared (NIR) light, for absorbance.

Results obtained indicated that the test samples that had been etched prior to soaking showed areas of silver-gray coloration. Controls not etched did not show any areas of coloration. The data show that NIR illumination of tooth samples contacted with a Gold probe detected early stage caries, including interproximal caries lesions.

### Example 4

### Detection of Caries in Human Subjects

Example herein describes a method of detecting early stage dental caries in a human subject using a tetracycline probe or a colloidal gold probe, respectively.

A dental patient is contacted via the buccal or lingual cavity with a solution of a tetracycline, such as a 1 mg/ml solution, as a gargle for 30 seconds. After extensive rinsing, the buccal or lingual cavity is illuminated with UV light, and respectively appearance and location of fluorescent spots, are probed with a hand-held attachment for a spectrophotometer. Areas of fluorescence or gray spots are photographed.

A dental patient is contacted via the buccal or lingual cavity with a solution of a colloidal gold, such as the BioRad Total Protein Stain, catalog number 170-6527, in order to soak the caries lesion and access the solution into the lesion. After gargling and extensive rinsing with water, the colloidal gold treatment is followed by a 20 second to 30 second gargle with Silver stain Plus (Bio-Rad). The buccal or lingual cavity is illuminated with near infra-red (NIR) light, and appearance and location of areas of gray-silver staining are probed with a hand-held attachment for a spectrophotometer. Areas of stain are photographed.

### Example 5

### Remineralization in Human Subjects of Early Stage Caries Detected by Optical Properties

Following detection, procedures for remineralization are initiated at locations assessed as having early stage caries. For example, fluoride ions are introduced into the lesion by use of a topical gel or toothpaste formulated for this purpose. The remineralization treatments leading to restoration of integrity of the enamel induce precipitation of calcium and phosphate on crystals in the enamel that are partially demineralized.

### Example 6

### Assessment of Detection Probes Using Bovine Samples

Bovine teeth were obtained and sliced to obtain enamel samples. These enamel samples (having no dentin layer), were fabricated from these bovine teeth. A de-mineralized area was prepared by etching an area of the sliced enamel samples. The sliced enamel surface containing a de-mineralized area was contacted with concentrations of each of the probes. The surface was illuminated with wavelengths of excitation light and light of an emission wavelength was detected. Further, the absorbance of illuminating wavelengths was detected. Optical observations were obtained directly by eye, by spectrophotometer, or by camera such as photographing the tooth or indirectly by having the data sent from a device/sensor on an optical device to an imager on the optical device.

### Example 7

### Assessment of Detection Probes Using Interproximal Models of Extracted Human Teeth

Interproximal models of extracted human teeth (molars and premolars) were fabricated using silicon impression materials. Interproximal enamel caries at about 500 nm depth and at about 1.5 mm depth were prepared by using areas of interproximal contact regions. The dental preparations having synthetic caries were assessed by radiographic films (bitewings) and a microscope.

A cotton ball was saturated with the detection probe, and placed in contact with the region of the interproximal enamel caries. The bound probe was observed by illuminating over a varying set of different wavelengths of excitation light, followed by detecting light of fluorescent emissions. Furthermore, absorbance of the illuminating wavelengths was detected as appropriate for each probe. Optical observations for each probe are shown in Table 1.

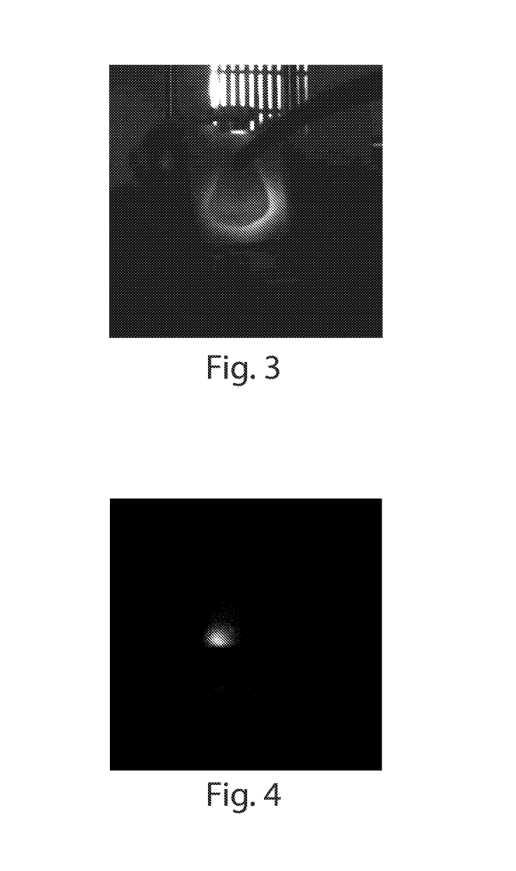

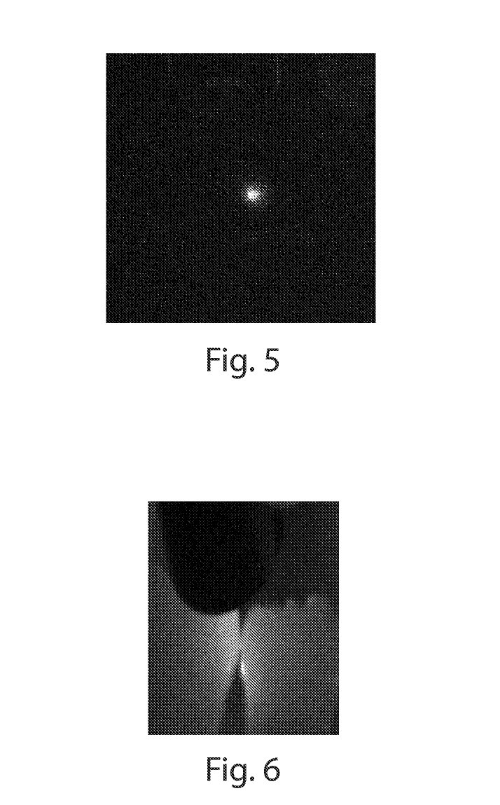

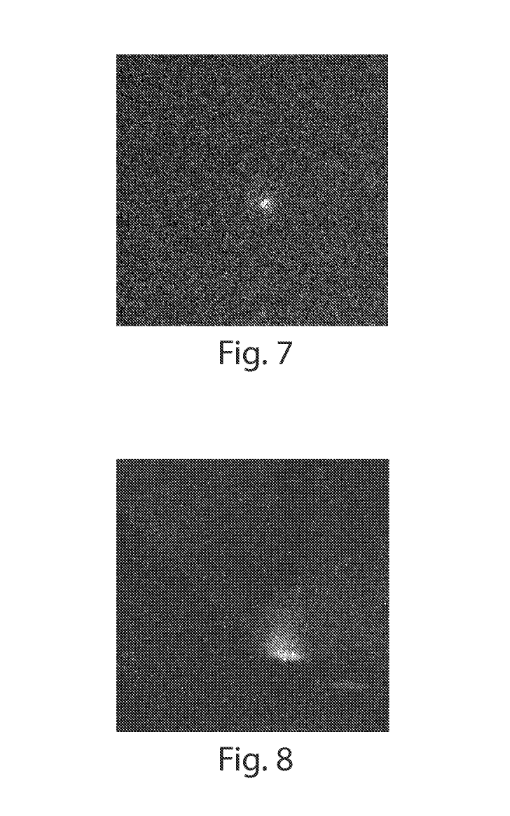

Each tooth preparation having interproximal enamel caries and bound probe was illuminated and photographed. Absorption of light was observed on a tooth preparation with an early stage caries lesion to which a preparation of a bismuth probe has been bound (FIG. 3). Fluorescence of an early stage caries lesion was observed in a tooth preparation to which a preparation of a Doxorubicin probe had been bound (FIG. 4), a Riboflavin probe had been bound (FIG. 5), a Chlorophyll A probe has been bound (FIG. 6), and a Porphyrin probe has been bound (FIG. 7). Bioluminescence of an early stage caries lesion was observed in a tooth preparation to which has been bound a probe preparation having luciferase and luciferin (FIG. 8).

### Example 8

### Determination of Variables

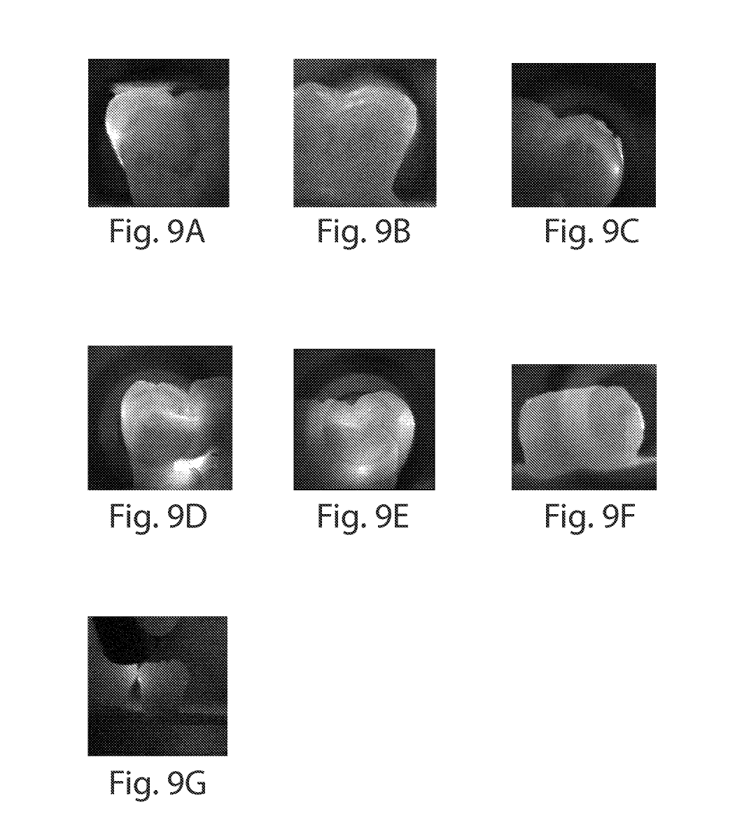

Probes were tested for appropriate concentration, kinetics of binding and retention. Intensity of the illumination light, and the size of the illumination light beam were varied, and data were recorded for each probe. Interproximal caries were observed by contacting caries with each of OsteoSense 750, AngioSense 750, Genhance 750, and Chlorophyll A, illuminating the tooth with light of an excitation wavelength, and detecting light of an emission wavelength (FIG. 9). The concentration of each probe was varied to determine an optimum concentration for binding during a time period suitable for dental use. Durations of contacting were also tested, for example about 10 seconds, about 20 seconds, about 40 seconds, or about 60 seconds. Binding of the probes to the surface of a tooth having different occlusal cavity conditions were also determined, e.g., plaque, gingival, calculus.

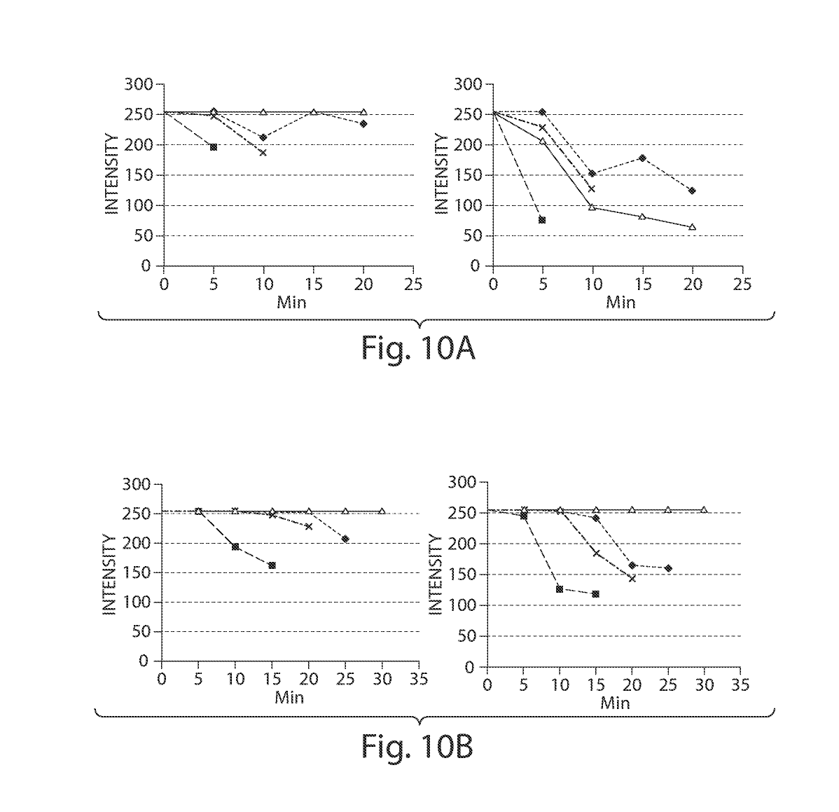

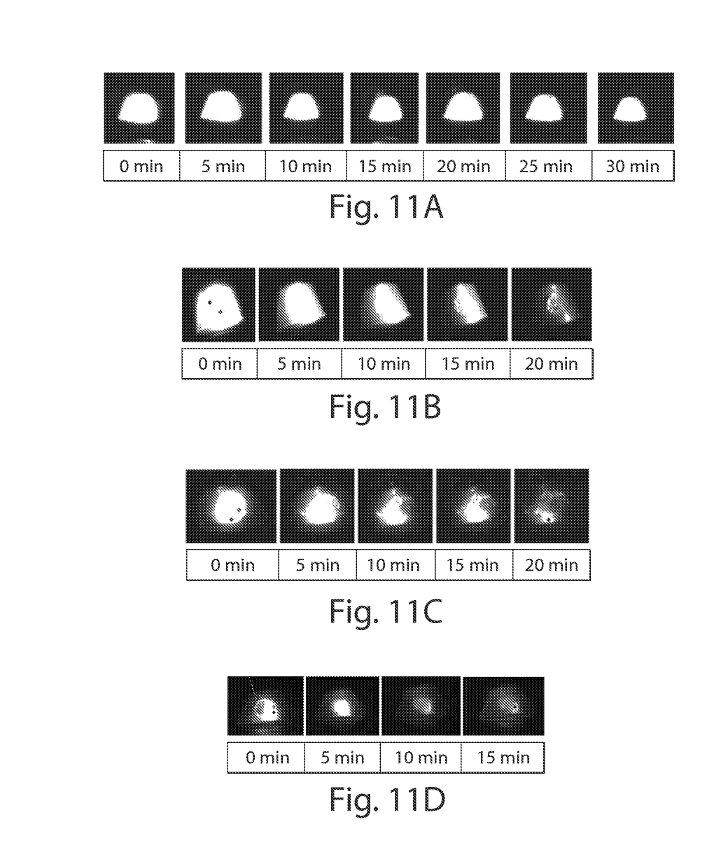

Data showed that multiple dilutions and concentrations of the probes could be used to bind and to detect early stage caries. Dilutions (1:10 and 1:100 dilutions from a 1 mg/mL stock solution) of each of CardioGreen (ICG), IR 820 (ICG), Cy7, and OsteoSense 750, and probe bound to caries were prepared, and tested for intensity of and duration of fluorescence. See FIG. 10A and FIG. 10B, and FIG. 11A-FIG. 11D.

TABLE 1
Properties of detection probes

| | | | excitation/ | | |
|---|---|---|---|---|---|
| | | | absorbance | emission | |
| | composition | | wavelength | wavelength | degree of |
| detection probe | type | detection type | (nm) | (nm) | detection |
| Bismuth | Metal | Absorbance | Absorption | | Good |
| Gold colloid | Metal colloid | Absorbance | 530 | | Weak |
| Doxorubicin | Anti-cancer | Fluorescence | 480 | 630 | Good |
| | medicine | | | | |
| Riboflavin | Vitamin B₂ | Fluorescence | 450 | 550-700 | Good |
| Chlorophyll A | Chlorophyll | Fluorescence | 614 | 670-900 | Good |
| Indocyanine Green | Dye | Fluorescence | 800 | 835 | Weak |
| Porphyrin | | Fluorescence | 600 | 700 | Good |
| Luciferase/ | Organic | Chemical | | 630 (PH) | Good |
| Luciferin | enzyme and | reaction | | dependent | |
| | protein | fluorescence | | | |
| Qdot | Nanodot | Fluorescence | 400-750 | 750-900 | Good |
| HiLyte Fluor | Small | Fluorescence | 720-750 | 750-800 | Good |
| | molecular | | | | |
| | weight | | | | |
| | compound | | | | |
| Bacterial | Chlorophyll | Fluorescence | 650-750 | 775-285 | |
| Chlorophyll | | | | | |
| OsteoSense 750 | Dye | Fluorescence | 740-760 | 770-790 | Very Good |
| CardioGreen (ICG) | Dye | Fluorescence | 750-800 | 820-870 | Good |
| IR820 (ICG) | Dye | Fluorescence | 745 | 845 | Good |
| Cy7 | Dye | Fluorescence | 700-770 | 760-800 | |
| Far-Green two | Dye | Fluorescence | | | |
| AngioSense 750 | Dye | Fluorescence | 740-760 | 770-790 | Weak |
| Genhance 750 | Dye | Fluorescence | 740-760 | 770-780 | Weak |
| AngioSpark 750 | Dye | Fluorescence | 740-760 | 770-790 | Weak |
| Alexa Fluor 750 | Dye | Fluorescence | 710-730 | 760-780 | Good |

It was observed from the data that images of Cy7 diluted 1:10 and images of CardioGreen diluted 1:10 showed similar fluorescence between 0 min and 15 min on both shallow and deep lesions. Images of Cy7 diluted 1:10 retained much greater intensity in both deep and shallow lesions compared to images of CardioGreen (ICG) diluted 1:10. Images of CardioGreen (ICG) and images of Cy7 were observed to produce greater fluorescent intensity at each concentration and lesion depth compared to IR820 (ICG). OsteoSense 750 diluted 1:100 was observed to produce greater intensity than IR820 even when diluted 1:10 (FIG. 11A-FIG. 11D).

### Example 9

### Application to Caries Using a Wedge for Delivery

The Example herein shows a probe was applied to a tooth having an interproximal caries lesion using a delivery device and the probe with bound caries was detected optically.

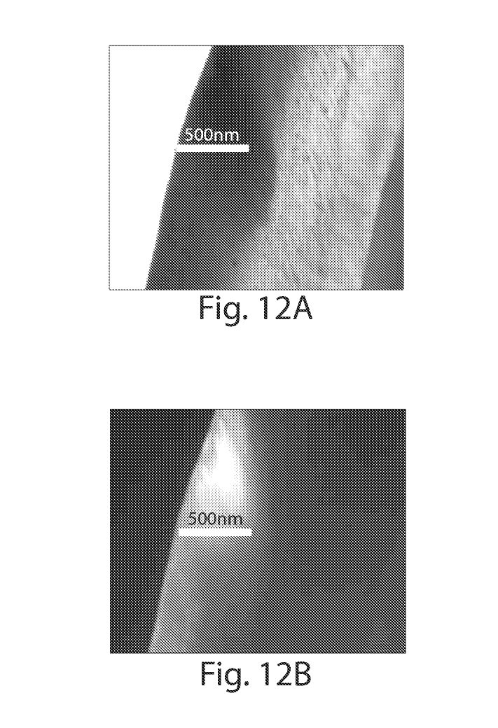

A fluorescence probe was contacted/inserted into a caries lesion using a wedge type of delivery tool. The probe was placed on the outside of the wedge and was then inserted between the teeth. Data shows that probe was successfully infiltrated into almost the entire area of the caries (compare FIG. 12A and FIG. 12B).

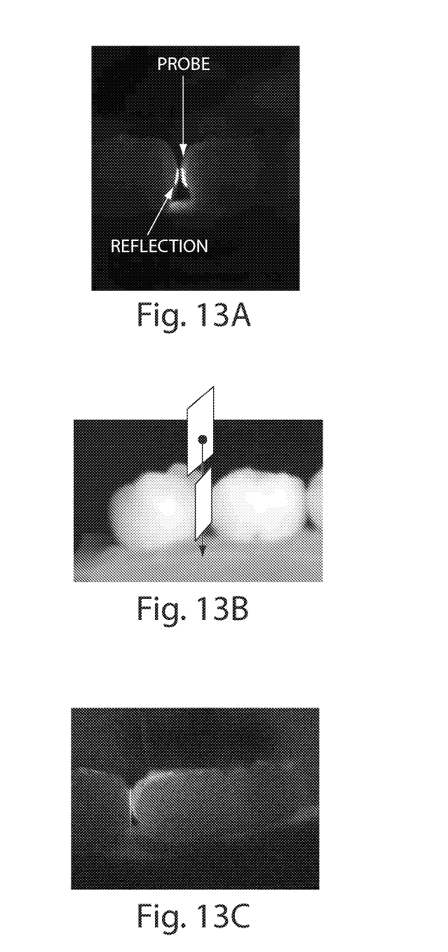

Fluorescence was observed on an adjacent tooth because of the reflection of a fluorescence of a tooth having a fluorescent probe bound to a caries lesion (FIG. 13A). A black/gray separator was placed between the teeth and it was observed that the separator eliminated reflection of fluorescence to the tooth not having an early stage dental caries (FIG. 13B and FIG. 13C).

### Example 10

### Removal of Probe from Two Model Systems: Extracted Teeth and Hydroxyapatite

Methods were tested for removing OsteoSense 750 bound to a tooth or to hydroxyapatite. The efficacy of removal of the probe by the solution was determined by criteria such as extent of fluorescence remaining on a tooth after rinse, or fluorescence extracted from hydroxyapatite into a rinse solution.

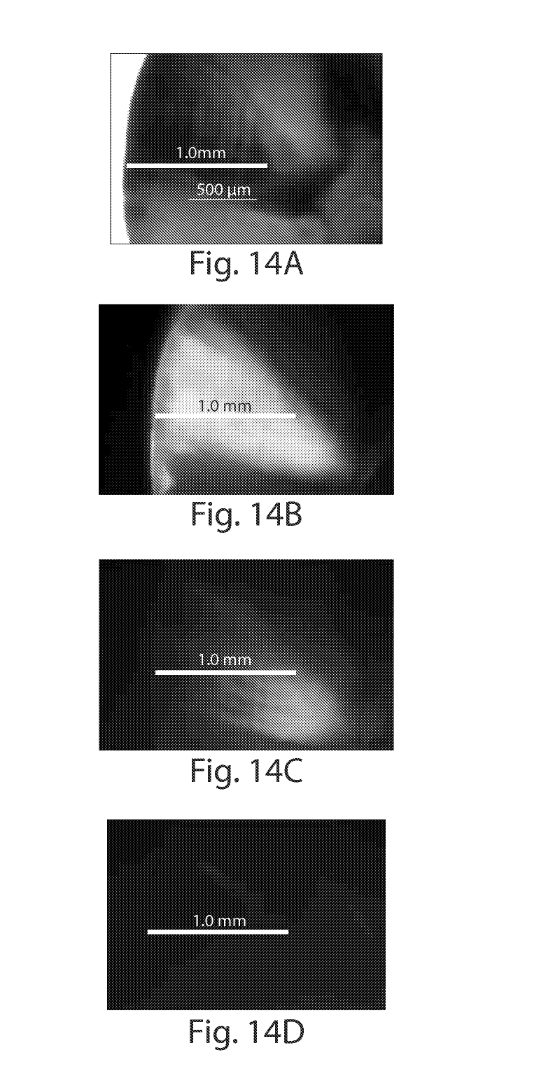

A caries lesion was identified on a tooth by light microscopy (FIG. 14A) and the optical detection system using OsteoSense 750 and fluorescence microscopy (FIG. 14B). The tooth was rinsed with a solution of hydrogen peroxide (38%, Ultradent Products Inc.). Three minutes after application of the hydrogen peroxide, the fluorescence of the probe bound to the tooth was observed to be substantially reduced (FIG. 14C). Ten minutes after application of the solution of hydrogen peroxide, fluorescence indicating presence of the bound probe was substantially eliminated (FIG. 14D). Thus, hydrogen peroxide was shown to be effective for removing OsteoSense 750 bound to caries.

Samples of hydroxyapatite (HA) and a volume of OsteoSense 750 were mixed in a tube. Solutions of each of hydrogen peroxide (30%), phosphoric acid (40%), or control, water, were added to a sample in a tube. The tubes were vortexed, and the mixture was allowed to settle to obtain a supernatant. Each supernatant was collected and analyzed for probe content using a spectrophotomer. A spectrometric signal at about 750 nm indicates a release of OsteoSense 750 that was bound to the HA. Supernatants from each of the water control and from hydrogen peroxide were observed to result in no signal at 750 nm. Data showed that the phosphoric acid solution supernatant resulted in a definite peak at 750 nm. Thus, data show that phosphoric acid was effective for removing OsteoSense 750 from hydroxyapatite.

Another method using hydrogen peroxide for removal of probe was tested in this Example, rinsing the hydroxyapatite or the tooth with saline (PBS) prior to treatment with hydrogen peroxide, and measuring absorbance to determine OsteoSense 750 removal at each step.

The efficacy of hydrogen peroxide was determined by comparing absorbance values at 750 nm for hydrogen peroxide rinse solutions, phosphate buffered saline (PBS) wash solutions, and control solutions. An absorbance at 750 nm within a spectrophotometric scan of 550 nm to 850 nm indicates a presence of OsteoSense 750 in each mixture. A control absorbance value at 750 nm for OsteoSense 750 diluted 1:100 in PBS was shown to be 0.337 and was used as a standard for purposes of calculations.

A sample of hydroxyapatite was contacted in a tube with a volume of OsteoSense 750 (1:100 dilution in PBS). The HA sample was washed with PBS and a spectrophotometric scan of the wash solution was performed. The absorbance at 750 nm was 0.036 for the wash solution. The HA sample was rinsed with a volume of hydrogen peroxide and a spectrophotometric scan of the hydrogen peroxide rinse solution was performed. The absorbance at 750 nm was 0.252 for the hydrogen peroxide fluid. A spectrophotometric scan was performed for a control solution of OsteoSense 750 diluted 1:100 in hydrogen peroxide, the absorbance value at 750 nm was 0.273.

A comparison of the absorbance values at 750 nm showed the presence of greater amounts of OsteoSense 750 in the hydrogen peroxide fluid compared to the PBS washes. Thus, hydrogen peroxide was effective for removing OsteoSense 750 that was bound to hydroxyapatite.

To further determine the efficacy of hydrogen peroxide (38%, Ultradent Products Inc.) for removing OsteoSense 750, five teeth having a caries lesion were contacted with a volume (1 μL) of OsteoSense 750, applied to a caries present on each tooth. The OsteoSense 750 (0.5 μL) was then collected from each tooth surface, and added to 100 μL of de-ionized water. A spectrophotometric scan was performed for each of these OsteoSense 750 solutions to determine binding differentially. Each tooth was then wiped with material to remove residual OsteoSense 750 from the tooth, and washed twice with PBS (150 μL volume for each wash).

The first and the second wash solution for each tooth were collected and analyzed using spectrophotometric scans, absorbance values at 750 nm were observed for the first PBS wash solutions. Absorbance values showed no probe present in the second PBS wash solutions.

A volume of hydrogen peroxide (2 μL of a 1:20 dilution in de-ionized water) was applied for one minute to the area of the tooth having the caries, 150 μL of PBS was added to each of the areas, and the hydrogen peroxide mixture was collected. Spectrophotometric scans were performed on these mixtures and showed background absorbance at 550 nm and at 850 nm. A representative absorbance value at 550 nm was subtracted from absorbance values at 750 nm for each hydrogen peroxide mixture.

Data analyses of the absorbance data at 750 nm included calculating absorbance of OsteoSense 750 after staining teeth, after uptake in caries, in PBS washes, and in hydrogen peroxide mixtures. The decay/reduction of absorbance values at 750 nm resulting from applying hydrogen peroxide to OsteoSense 750, and corrected absorbance values were calculated. Data show that hydrogen peroxide removed 80% to 93% of OsteoSense 750 from the caries (Table 2).

Thus, data show that hydrogen peroxide was effective for removing OsteoSense 750 bound to hydroxyapatite and OsteoSense 750 bound to caries on teeth.

### Example 11

### Sensitivity, Specificity, Predictive Value of the Optical Detection System

This example was performed to determine whether the optical detection system herein provides constant and reliable results for accurate representation of caries depth and location.

Data were obtained from 33 extracted human permanent premolars and molars characterized as free of restorations. These 33 teeth were found to contain ten white spots and eleven cavitated lesions. Twelve teeth were free of lesions.

An interproximal surface of each tooth was used for this example and was numbered in a random order. OsteoSense 750 (1 μl of 1:100 dilution) was applied to the interproximal areas of each tooth for 10 seconds, and teeth were washed with distilled water and gently wiped with a gauze (2×2).

The optical devices were: NIR light source (Xenon light source MAX 301, Asahi Spector, Japan) with a filter that produces an excitation wavelength of about 740 nm; CCD detector camera (MC285SPD-L0B0, Texas Instrument, USA) that receives light through a 790 nm filter to detect fluorescent emission wavelength at about that wavelength; platform to stabilize specimens (Suruga, Japan); and image analysis software (Capture Base, Japan).

NIR light (740 nm) was used to illuminate teeth from two different directions. Teeth were illuminated by NIR light either indirectly by exciting the interproximal area through the occlusal layer of enamel (FO) or directly by exciting the interproximal area (FI). Emission light signals were then captured from the buccal surface of teeth with a detector-filter (790 nm).

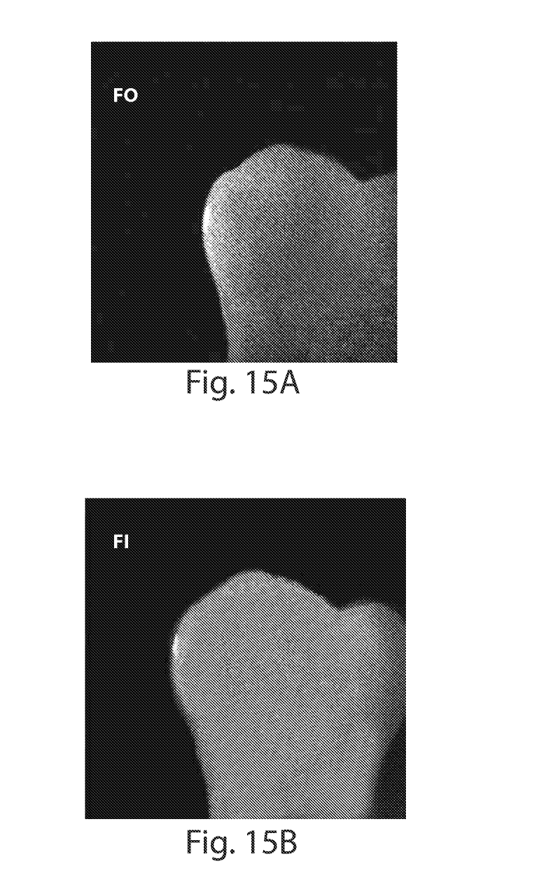

Representative data are shown in FIG. 15A-FIG. 15B. Bitewing X-ray images of the teeth were obtained in addition to the occlusal illumination and interproximal illumination images. Data were obtained from eleven dentists who evaluated the X-ray, occlusal illumination and interproximal illumination images, and each assessment for a tooth was recorded either as a No Lesion or a Lesion.

TABLE 2
Absorbance data (750 nm) showing efficacy of hydrogen peroxide to remove OsteoSense 750 from teeth having caries

| | Absorbance at 750 nm | | | | |
|---|---|---|---|---|---|
| Solution | tooth A | tooth B | tooth C | tooth D | tooth E |
| OsteoSense OD750 after stained tooth^{a} | 0.3438 | 0.2986 | 0.1562 | 0.242 | 0.2574 |
| OsteoSense 750 uptake in caries^{b} | 0.1836 | 0.2288 | 0.3712 | 0.2854 | 0.27 |
| PBS wash^{c} | 0.01245 | 0.0087 | 0.0135 | 0.0195 | 0.00525 |
| hydrogen peroxide rinse^{d} | 0.0246 | 0.0378 | 0.01425 | 0.0138 | 0.01395 |
| decay of OsteoSense750 at 1 min by hydrogen peroxide^{c} | 0.0727/0.3989 = 1/5.49 | | | | |
| corrected absorbance of hydrogen peroxide mixture^{f} | 0.135054 | 0.207522 | 0.078233 | 0.075762 | 0.076586 |
| % OsteoSense 750 removed^{g} | 80 | 80 | 93 | 88 | 93 |
| OD750 of OsteoSense750 (1 μl in 100 μl dw^{h}) | | | | | |
| OsteoSense750 uptake in cariesb is h minus a | 0.5274 | | | | |
| hydrogen peroxide absorbance correction is d times 5.49 | | | | | |
| % washed (g) is 100 minus (100 times ((b plus f) divided by a) | | | | | |

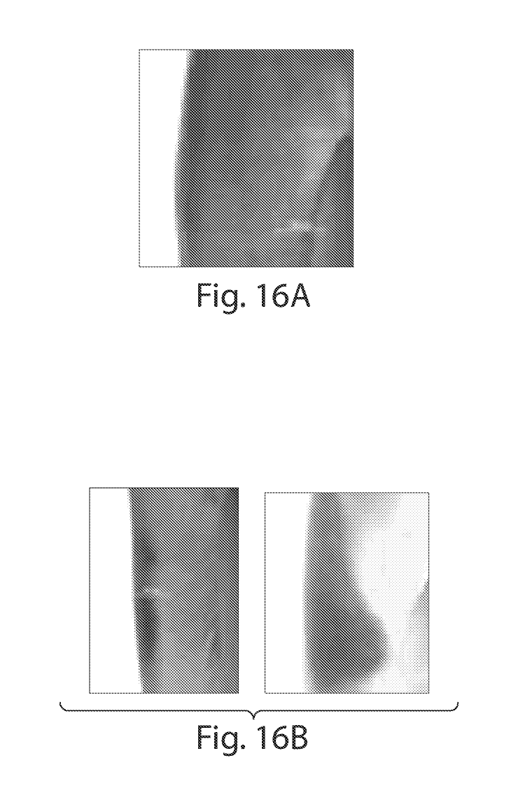

Each tooth was then prepared and polished using Vector Powerhead (Buehler, USA) from the buccal and lingual surfaces into 1 mm sections. Each surface was then examined by light microscope (Four-fold magnification; Olympus, USA) for histological examination, which is recognized as a gold standard method by present clinicians to determine the presence of caries in teeth. Using the histological examination, the teeth were recorded in categories as No lesion or Lesion. FIG. 16A and FIG. 16B show representative histological data.

Sensitivity, specificity, positive predictive value (PPV), and negative predictive value (NPV) were calculated for each of the three methods using the data from these determinations. Assessment by the evaluators of the three methods was compared with the results obtained by histological examination. Lesions were observed to be present in 23 of 33 interproximal surfaces as determined by histological examination.

The data in Table 3 showed that the optical detection system described herein is characterized by higher sensitivity and NPV that those of the other method, X-rays. Sensitivity and NPV were observed to be, for each type of imaging, statistically significant. The specificity and PPV between any two methods were found to be statistically insignificant.

TABLE 3
Statistical analysis: sensitivity, specificity, PPV and NPV

| method | | | | |
|---|---|---|---|---|
| (abbreviation) | sensitivity* | specificity | PPV | NPV* |
| X-ray (XR) | 0.32 | 0.845 | 0.853 | 0.348 |
| | (0.20, 0.44) | (0.78, 0.91) | (0.43, 1.00) | (0.18, 0.52) |
| Occlusal | 0.715 | 0.9 | 0.947 | 0.584 |
| illumination | (0.56, 0.87) | (0.76, 1.00) | (0.87, 1.00) | (0.35, 0.82) |
| (FO) | | | | |
| Interproximal | 0.893 | 0.764 | 0.9 | 0.775 |
| illumination | (0.81, 0.98) | (0.57, 0.95) | (0.80, 1.00) | (0.57, 0.98) |
| (FI) | | | | |

*indicates statistical significance

The inter-examiner reliability (Fleiss' kappa values) of each of occlusal illumination and interproximal illumination, respectively, was 0.77 and 0.66. This compares to inter-examiner reliability of 0.28 for X-rays. The data showed that there was less variability in the evaluators' determinations of caries presence on a tooth using the optical detection systems described herein compared to bitewing X-rays. These data show that clinicians using this optical detection system will be able to make a best possible diagnosis compared to other systems.

### Example 12

### Early Caries Detection Performance of the Optical Detection System

The ability of the caries detection system to detect early stage caries was assessed using 24 extracted human permanent premolars and molars which had a white spot. One interproximal surface of each tooth was used for the experiment and surfaces were numbered randomly. OsteoSense 750 (1 μl of 1:100 dilution) was applied to the interproximal areas of each tooth for 10 seconds, and these areas were washed with distilled water and gently wiped with a gauze. The optical device system used to examine the teeth was described in Example 11.

NIR light (740 nm) illuminated teeth from two different directions, occlusal illumination, FO, or interproximal illumination, FI. Emission light signal were then captured from the buccal surface of teeth with a detector-filter (790 nm). X-ray images of the teeth were also obtained.

Data were obtained from six dentists who evaluated the X-ray, occlusal illumination and interproximal illumination images, and each assessment was recorded either as a No lesion, or as a Lesion, with a Lesion being less than one-half enamel depth, or a Lesion with more than one-half enamel depth.

Histological examination of the teeth was performed for each tooth and determined that the 24 teeth had caries with demineralization that extended less than one-half depth of the enamel, i.e., caries lesion (FIG. 16A and FIG. 16B).

Each assessment was categorized into the following statistical categories: a Match, an underestimation (UE) or an overestimation (OE), by comparing the result to the histological results. Histological examination determined that all the teeth had early caries, defined as lesions with a depth less than one-half enamel depth. Thus, an assessment using X-ray, occlusal illumination, and interproximal illumination images of a lesion with depth less than one-half enamel depth was categorized as a Match. Assessment of no lesion by X-rays, occlusal illumination, and interproximal illumination was categorized as an underestimation (UE) of the true lesion size by histological examination, and X-rays, occlusal illumination, and interproximal illumination assessment of Lesion with depth more than one-half enamel depth was categorized as an over estimation (OE) of the true lesion size by histological examination.

Statistical analysis was performed using the SAS version 9.1 statistical package (SAS Institute, USA). A formal comparison of the diagnostic methods was achieved using a Mixed-effects multinominal logistic regression model. See Table 4 and Table 5.

A statistically significant difference in the odds ratios was observed for an underestimated (UE) misclassification compared to a Match for observations by evaluators of occlusal illumination images or interproximal illumination images compared to X-ray images. There was no statistically significant difference in the odds ratios for an overestimated (OE) misclassification compared to a Match when examiners used occlusal illumination or interproximal illumination images compared to X-ray images. The inter-examiner reliability (Fleiss' kappa values) for occlusal illumination was calculated to be 0.68 and for interproximal illumination was calculated to be 0.51 compared to only 0.15 for X-rays. The overestimated errors were observed to be statistically insignificant between the systems. These data show that the methods of optical detection herein are significantly improved with respect to underestimated errors compared to traditional X-rays and that early stage dental caries were successfully detected.

TABLE 4
Mean values and standard deviations of the X-rays, occlusal
illumination and interproximal illumination

| method | | | |
|---|---|---|---|
| (abbreviation) | Match | Underestimation | Overestimation |
| X-ray (XR) | 11.81 (7.18) | 79.17 (13.18) | 9.03 (8.09) |
| Occlusal | 29.17 (9.50) | 53.47 (8.09) | 17.36 (4.10) |
| illumination (FO) | | | |
| Interproximal | 64.58 (9.03) | 13.89 (6.80) | 21.53 (4.87) |
| illumination (FI) | | | |

TABLE 5
Statistical analysis of X-ray, occlusal illumination, and interproximal
illumination images as compared to histological examination

| outcome | | odds ratio for | |
|---|---|---|---|
| categories | | comparing two | P |
| compared | methods compared | methods | value |
| UE* and Match | Occlusal illumination and | 0.2703 | 0.0100* |
| | X-ray | | |
| | Interproximal illumination | 0.0309 | 0.0002* |
| | and X-ray | | |
| OE* and Match | Occlusal illumination and | 0.7697 | 0.5843 |
| | X-ray | | |
| | Interproximal illumination | 0.4205 | 0.0975 |
| | and X-ray | | |

*indicates statistical significance

### Example 13

### Comparison of the Size of a Caries Lesion Identified by a Probe to Actual Size of the Caries Lesion

Data were obtained to determine the accuracy between by the optical detection system as compared to the true depth of a caries lesion as determined by a standard method.

A five millimeter section was obtained from each of 33 extracted human teeth by grinding from the buccal and/or lingual side. OsteoSense 750 (1 μl of 1:100 dilution) was applied to the interproximal areas of each section for 10 seconds. These interproximal areas were washed with distilled water and gently wiped with gauze. Light microscope images of the teeth were obtained. The teeth were illuminated with NIR excitation light (740 nm) and the fluorescent signal from the carious lesions were detected and pictured using a fluorescent microscope with an emission filter (790 nm).

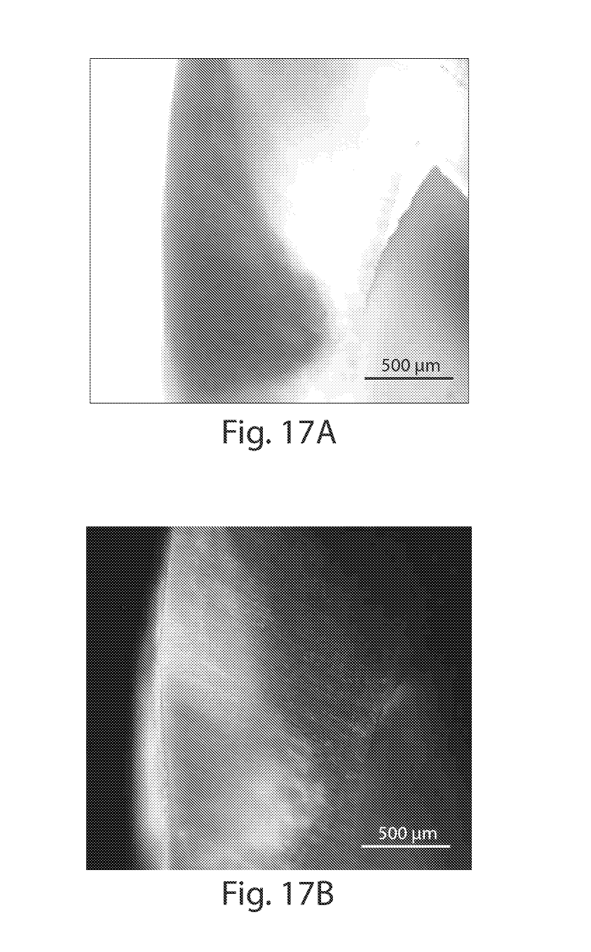

The fluorescence microscope data showed the depth determined by OsteoSense 750 in the optical detection system, while the light microscope showed the actual caries depth (or the best means of measuring the true depth). A representative set of teeth analyzed in this manner is shown in FIG. 17A and FIG. 17B. Analysis of the images showed that the depth of the lesions was 0.2 mm to 1.5 mm, the average filtration extent was 93.72±14.5% and the correlation coefficient was −0.002.

Data obtained in this example showed that the optical detection system delineated about 93% of the true size of the caries and that there was no significant correlation between infiltration extent and the depth of the carious lesion. Thus, the optical detection system using OsteoSense 750 was shown to be effective in identifying the true depth of caries in human extracted teeth.

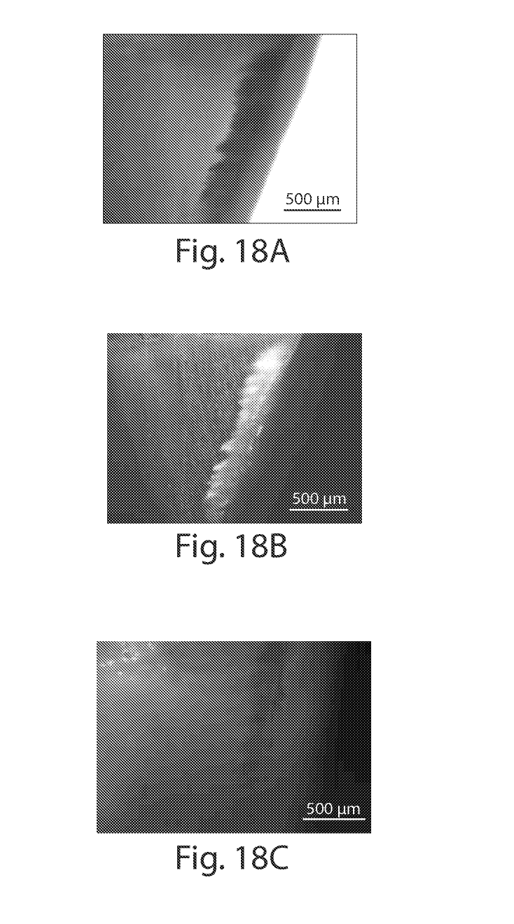

Images of teeth with caries lesions bound to OsteoSense 750 were taken using a light microscope and the optical detection system. Hydrogen peroxide (38%, Ultradent Products Inc.) was applied with a syringe to the caries lesion. This solution was observed to have eliminated fluorescence (FIG. 18A-FIG. 18C). Thus, hydrogen peroxide was found to be effective for removing a fluorescent probe bound to caries lesions on human teeth as detected using the optical system.

### Example 14

### RFU Comparison of Efficacy of Solutions to Release from OsteoSense 750 from HA

A comparison of efficacy of solutions of various agents to release OsteoSense 750 from hydroxyapatite was tested. Variables included conditions of pH, temperature, and presence of 1% calcium chloride. Prior to testing the efficacy of the solutions to release OsteoSense 750 from hydroxyapatite, a standard curve was prepared to obtain a linear region for correlation of relative fluorescence value with concentration of OsteoSense 750.

The standard curve was prepared using dilutions of OsteoSense 750 (1:100, 1:200, 1:400, 1:800, and 1:1000 of a 1 mg/mL stock solution), diluted in wells; a volume (90 μL) of each was collected. and fluorescence was measured using a fluorometer. The observed relative fluorescence units (RFU) were plotted as a function of a relative concentration (1:1000 to 1:100), and a linear relationship was observed. Relative fluorescence units observed in amounts were from about 10 at the 1:1000 dilution to about 110 at the 1:100 dilution. Further, the RFU of 1:300 dilution was observed to be 37.5 RFU. Further data obtained from examples herein used this curve to obtain amounts, and examples included a standard dilution of 1:300 of OsteoSense 750.

To compare efficacy of various agents to release OsteoSense 750 from hydroxyapatite, a sample of HA was mixed with OsteoSense 750 (1:300). Solutions of 1%, 5%, 10%, and 20% of phosphoric acid (PA), sodium phosphate monobasic (M), sodium phosphate dibasic (D), and methylene phosphoric acid (MDP) were each added to each of four replicates of the HA mixed with OsteoSense 750. Three conditions were tested: incubation for 15 minutes at 37° C. after vortexing; adjusting pH to 6.5 at 23° C. after vortexing; and addition of 1% calcium chloride to each solution and incubation at room temperature after vortexing. A raw RFU value was determined for each supernatant using a fluorometer, and amount of OsteoSense 750 released was calculated as a percent of total OsteoSense 750 or total reference value. See Tables 6, 7 and 8.

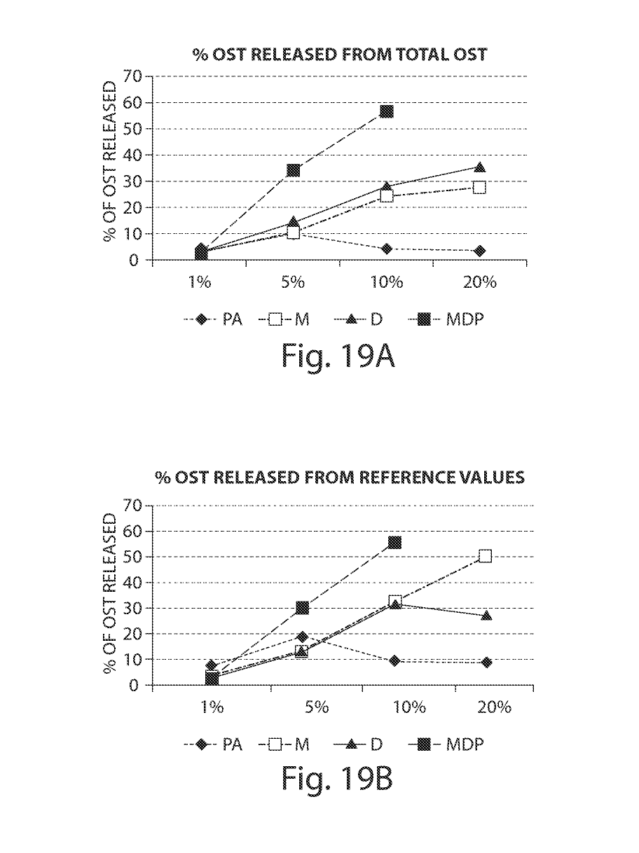

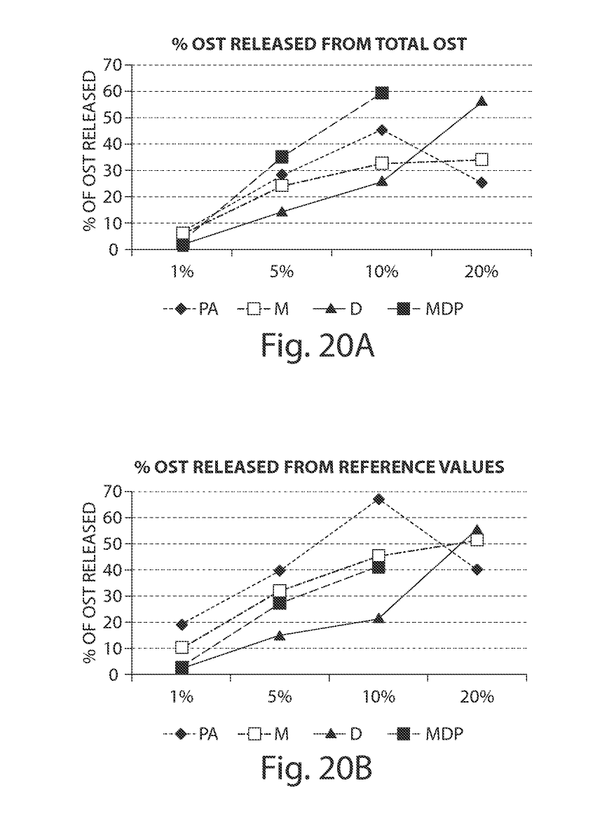

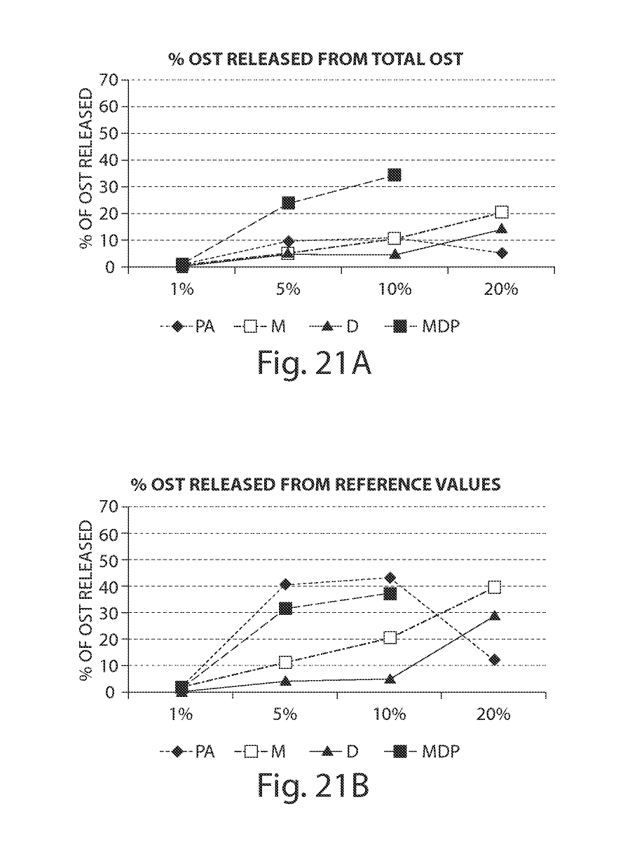

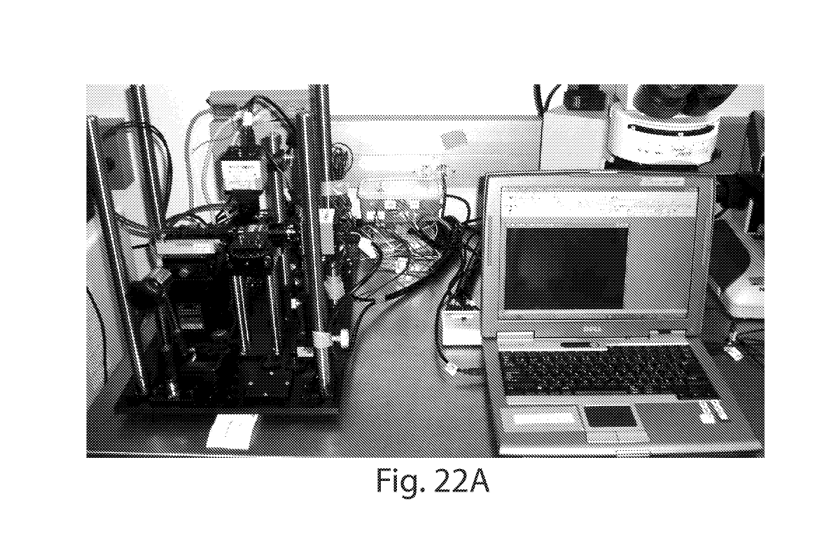

It was observed that the amount of OsteoSense 750 (Ost) released from HA was greater at higher concentrations for each of sodium phosphate monobasic, sodium phosphate dibasic, and methylene phosphoric acid solutions, each of which was incubated for 15 minutes at 37° C. (FIG. 19A and FIG. 19B). Phosphoric acid was observed to dissolve the HA. Methylene phosphoric acid was observed to remove more OsteoSense 750 from HA than sodium phosphate monobasic or sodium phosphate dibasic at each concentration (FIG. 19A and FIG. 19B). At pH 6.5, methylene phosphoric acid released more OsteoSense 750 than other agents. Phosphoric acid at pH 6.5 was effective at releasing OsteoSense 750 (FIG. 20A and FIG. 20B). Each of methylene phosphoric acid and phosphoric acid in calcium chloride (1%) were shown to be most effective in releasing OsteoSense 750 from HA (FIG. 21A and FIG. 21B).

TABLE 6
Removal of OsteoSense 750 by incubation for 15 minutes at 37° C.

| agent | | 1% | 5% | 10% | 20% |
|---|---|---|---|---|---|
| (abbreviation) | | solution | solution | solution | solution |
| phosphoric | Raw RFU value | 1.73 | 3.67 | 1.64 | 1.33 |
| acid (PA) | % released of | 4.61 | 9.78 | 4.37 | 3.54 |
| | total OsteoSense | | | | |
| | % released of | 7.36 | 18.94 | 9.10 | 8.51 |
| | total reference | | | | |
| | value | | | | |
| sodium | Raw RFU value | 1.16 | 3.90 | 8.97 | 10.37 |
| phosphate | % released of | 3.10 | 10.41 | 23.92 | 27.67 |
| monobasic (M) | total OsteoSense | | | | |
| | % released of | 3.65 | 13.11 | 32.36 | 50.11 |
| | total reference | | | | |
| | value | | | | |
| sodium | Raw RFU value | 1.11 | 5.33 | 10.46 | 13.30 |
| phosphate | % released of | 2.97 | 14.21 | 27.90 | 35.48 |
| dibasic (D) | total OsteoSense | | | | |
| | % released of | 2.30 | 13.12 | 31.69 | 27.01 |
| | total reference | | | | |
| | value | | | | |
| methylene | Raw RFU value | 0.93 | 12.88 | 21.21 | −0.05 |
| phosphoric | % released of | 2.49 | 34.33 | 56.56 | −0.13 |
| acid (MDP) | total OsteoSense | | | | |
| | % released of | 2.11 | 30.09 | 55.84 | n.d. |
| | total reference | | | | |
| | value | | | | |

n.d. indicates not determined

TABLE 7
Removal of OsteoSense 750 with solutions
at pH 6.5, incubation at room temperature

| agent | | 1% | 5% | 10% | 20% |
|---|---|---|---|---|---|
| (abbreviation) | | solution | solution | solution | solution |
| phosphoric | Raw RFU value | 2.31 | 10.64 | 17.00 | 9.50 |
| acid (PA) | % released of | 6.17 | 28.38 | 45.32 | 25.34 |
| | total OsteoSense | | | | |
| | % released of | 18.74 | 39.54 | 67.03 | 39.91 |
| | total reference | | | | |
| | value | | | | |
| sodium | Raw RFU value | 2.38 | 8.95 | 12.16 | 12.71 |
| phosphate | % released of | 6.36 | 23.87 | 32.44 | 33.90 |
| monobasic (M) | total OsteoSense | | | | |
| | % released of | 9.73 | 31.74 | 45.17 | 51.43 |
| | total reference | | | | |
| | value | | | | |
| sodium | Raw RFU value | 0.68 | 5.49 | 9.66 | 21.07 |
| phosphate | % released of | 1.81 | 14.64 | 25.75 | 56.19 |
| dibasic (D) | total OsteoSense | | | | |
| | % released of | 2.08 | 14.63 | 21.10 | 55.57 |
| | total reference | | | | |
| | value | | | | |
| methylene | Raw RFU value | 1.35 | 13.35 | 22.41 | n.d |
| phosphoric | % released of | 3.59 | 35.61 | 59.77 | n.d |
| acid (MDP) | total OsteoSense | | | | |
| | % released of | 2.50 | 27.41 | 41.32 | n.d |
| | total reference | | | | |
| | value | | | | |

n.d. indicates not determined

TABLE 8
Removal of OsteoSense 750 with calcium chloride (1%) addition
to each solution, and incubation at room temperature

| agent | | 1% | 5% | 10% | 20% |
|---|---|---|---|---|---|
| (abbreviation) | | solution | solution | solution | solution |
| phosphoric | Raw RFU value | 0.38 | 3.54 | 3.97 | 1.92 |
| acid (PA) | % released of | 1.01 | 9.44 | 10.57 | 5.12 |
| | total OsteoSense | | | | |
| | % released of | 2.27 | 40.79 | 43.29 | 11.99 |
| | total reference | | | | |
| | valve | | | | |
| sodium | Raw RFU value | 0.22 | 2.00 | 3.85 | 7.51 |
| phosphate | % released of | 0.60 | 5.34 | 10.27 | 20.04 |
| monobasic (M) | total OsteoSense | | | | |
| | % released of | 2.51 | 11.39 | 20.25 | 39.19 |
| | total reference | | | | |
| | valve | | | | |
| sodium | Raw RFU value | 0.00 | 1.75 | 1.84 | 5.26 |
| phosphate | % released of | 0.01 | 4.67 | 4.90 | 14.02 |
| dibasic (D) | total OsteoSense | | | | |
| | % released of | 0.02 | 4.11 | 5.28 | 29.08 |
| | total reference | | | | |
| | valve | | | | |
| methylene | Raw RFU value | 0.54 | 8.96 | 12.83 | n.d |
| phosphoric | % released of | 1.45 | 23.89 | 34.23 | n.d |
| acid (MDP) | total OsteoSense | | | | |
| | % released of | 1.54 | 32.07 | 37.57 | n.d |
| | total reference | | | | |
| | value | | | | |

n.d. indicates not determined.

The examples herein show several agents and conditions were effective for removal of OsteoSense 750 from HA. Methylene phosphoric acid was generally most effective for removing OsteoSense 750.

### Example 15

### Data Analysis: Sensitivity, Specificity and User Reliability

The thirty-three teeth specimens from Example 11 were analyzed by histological examination. Lesions were present in 23 of the 33 total teeth. The overall sensitivity, specificity, positive predictive value (PPV), and negative predictive value (NPV) for occlusal illumination (FO), and interproximal illumination (FI) were calculated and compared to data taken from X-rays (XR; Table 3). Standard errors were calculated using bootstrap, a nonparametric approach for evaluating the distribution of a statistic based on random re-sampling. Statistical analyses were performed using SAS version 9.1 statistical package (SAS Institute; Cary, N.C.) The user reliability, a parameter designated “inter-examiner” reliability, for each detection image obtained by occlusal illumination, interproximal illumination, and X-rays was calculated (Cohen's kappa values and Fleiss' kappa values).

Mean sensitivity, specificity, PPV, NPV and corresponding 95% confidence intervals for each of these detection methods, occlusal illumination and interproximal illumination, is shown in Table 3 in comparison to X-rays. A significant difference in sensitivity was observed between assessments and identifications using each of occlusal illumination images and X-ray images (p<0.0001), between assessments using each of interproximal illumination images and X-ray images (p<0.0001), and between assessments using each of interproximal illumination images and occlusal illumination images (p=0.00656). A significant difference in NPV between assessments was observed between data for the occlusal illumination images and the X-ray images (p<0.0004); between data for the interproximal illumination images and the X-ray images (p<0.0001); and between data for the interproximal illumination images and the occlusal illumination images (p=0.031). No statistically significant differences in specificity and PPV were observed among the occlusal illumination, the interproximal illumination and the X-ray images.

Increased inter-examiner reliability was observed using the occlusal illumination and the interproximal illumination methods compared to the conventional radiological X-ray method. A larger kappa value corresponds to an increased indication of consistency and repeatability.

Cohen's kappa was calculated for each of the results using the images. The data in Table 8 show the most reliability for the occlusal illumination images compared to assessments using the other methods, based on dentists' assessments (greater kappa value) (Table 9).

TABLE 9
Inter-examiner reliability (Cohen's Kappa values) of X-
rays, occlusal illumination and interproximal illumination

| method | kappa | SD |
|---|---|---|
| X-ray (XR) | 0.066 | 0.264 |
| Occlusal illumination (FO) | 0.095 | 0.201 |
| Interproximal illumination (FI) | −0.105 | 0.215 |

The inter-examiner reliability from Fleiss' kappa values and 95% confidence interval was higher for assessments using the occlusal illumination images and the interproximal illumination images compared to assessments using the X-ray images (Table 10).

TABLE 10
Inter-examiner reliability (Fleiss' kappa values) of
X-rays, occlusal illumination and interproximal illumination

| method | kappa | 95% CI |
|---|---|---|
| X-ray (XR) | 0.284 | 0.238, 0.33 |
| Occlusal illumination (FO) | 0.768 | 0.722, 0.814 |
| Interproximal illumination (FI) | 0.66 | 0.614, 0.706 |

Occlusal illumination and interproximal illumination, the methods described herein, resulted in improved detection of dental caries compared to X-rays as shown by the statistical calculations in Tables 9 and 10. The occlusal illumination and interproximal illumination methods herein showed improved sensitivity, specificity and inter-examiner reliability.

### Example 16

### Statistical Analysis of Images Compared to Histological Examination

White spot lesions (WSL) were observed by histological examination in 24 of 26 teeth examined. See Example 12. The lesions in these teeth were observed to be located in a depth of less than half the enamel. These 24 teeth were assessed also using occlusal illumination and interproximal illumination images/methods described herein and compared to X-rays.

A total of 144 assessments were made by six dentists as evaluators of the 24 teeth using each of the three images/detection methods. Each assessment was categorized into a Match, an underestimation (UE) or an overestimation (OE) by comparing the result to the histological results. Histological examination data determined that all teeth had early caries, defined as lesions with a depth less than one-half enamel depth. Thus, an assessment using X-ray, occlusal illumination, and interproximal illumination images of a lesion with depth less than one-half enamel depth was categorized as a Match. Assessment of no lesion by X-rays, occlusal illumination, and interproximal illumination was categorized as an underestimation (UE) of the true lesion size by histological examination, and X-rays, occlusal illumination, and interproximal illumination assessment of Lesion with depth more than one-half enamel depth was categorized as an over estimation (OE) of the true lesion size by histological examination.

It was observed that the occlusal illumination and the interproximal illumination images/methods described herein resulted in increased detection of WSL compared to the X-ray images (Table 11). The data in Table 11 show that the dentists viewing the interproximal illumination images and the occlusal illumination images detected the highest number of WSL. Data indicate that dentists using the X-ray images scored a much higher number of incorrect No lesion assessments than the dentists using either of the methods of occlusal illumination and interproximal illumination described herein

TABLE 11
Examiners' assessments of numbers of lesions obtained
by each of X-ray, occlusal illumination and interproximal
illumination methods (total for each method: 144)

| | | Lesions less than | Lesions greater |
|---|---|---|---|
| | | one-half enamel | than one-half |
| method | No lesion | depth | enamel depth |
| X-ray (XR) | 114 | 17 | 13 |
| Occlusal | 77 | 42 | 25 |
| illumination (FO) | | | |
| Interproximal | 20 | 93 | 31 |
| illumination (FI) | | | |

Statistical analyses were performed for the images/detection methods of occlusal illumination and interproximal illumination and were compared to X-rays, using a mixed-effects multinominal logistic regression model (Table 12). Statistically significant differences in odds ratio were observed for both overall misclassification compared to a Match, and for underestimated (UE) misclassification compared to a Match, for data obtained from dentists who viewed the occlusal illumination images or the interproximal illumination images compared to the X-ray images. No significant differences were observed for overestimated (OE) misclassification compared to a Match for dentists that viewed these images/methods.

The odds ratio for an overall misclassification compared to a Match for the occlusal illumination images compared to X-ray images was 0.3215, for the interproximal illumination images compared to X-ray image was 0.0708, and for the interproximal illumination images compared to occlusal illumination images was 0.2203. Data analyses show that the odds of having an overall misclassification error were 68% lower for evaluators who viewed the occlusal illumination images in contrast to those who viewed the X-ray images, and were 93% lower for evaluators who viewed the interproximal illumination images in contrast to the X-ray images. It was also observed that the odds of having an overall misclassification error were 78% lower for evaluators who viewed the interproximal illumination images compared to the occlusal illumination images.

TABLE 12
Odds Ratios data for comparisons of X-rays, occlusal
illumination and interproximal illumination

| Odds ratios for | Outcome Category | | |
|---|---|---|---|
| comparing two | Overall Error and | UE error and | OE error and |
| methods | Match | Match | Match |
| occlusal | 0.3215 | 0.2703 | 0.7697 |
| illumination | (p = 0.0161*) | (p = 0.01*) | (p = 0.5843) |
| and X-rays | | | |
| interproximal | 0.0708 | 0.0309 | 0.4205 |
| illumination | (p = 0.0004*) | (p = 0.0002*) | (p = 0.0975) |
| and X-rays | | | |
| interproximal | 0.2203 | 0.1144 | 0.5463 |
| illumination | (p = 0.002*) | (p = 0.001*) | (p = 0.1259) |
| and occlusal | | | |
| illumination | | | |

*indicates statistical significance

The odds ratio for an underestimated misclassification in comparison to a Match for the occlusal illumination images compared to the X-ray images was 0.2703, for the interproximal illumination images compared to the X-ray images was 0.0309, and for the interproximal illumination images compared to the occlusal illumination images was 0.1144. Data analyses show that the odds of having an underestimated error were 73% lower for evaluators who viewed the occlusal illumination images compared to the X-ray images, and were 97% lower for evaluators who viewed the interproximal illumination images compared to the X-ray images. The odds of having an overall misclassification error were 89% lower for evaluators viewed who the interproximal illumination images compared to those who viewed the occlusal illumination images.

These data show that the occlusal illumination images and the interproximal illumination images yield improved results compared to the conventional X-rays. Improved sensitivity in detecting caries and negative predictive values was observed using either the occlusal illumination images or the interproximal illumination images compared to the X-ray images. Improved odds ratios for both an overall misclassification and an under-estimated misclassification compared to a Match was also observed for the occlusal illumination and the interproximal illumination methods compared to X-rays. These data indicate that the methods described herein more reliably detected early stage dental caries than X-rays. Caries can be detected and therefore remineralized, without having to perform procedures that are extremely invasive, costly and painful.

### Example 17

### Comparison of Caries Size Detected Using a Bisphosphonate-Cyanine Dye and Light Microscopy

Accuracy of an optical detection system using a bisphosphonate-fluorescent probe was compared to a standard method that uses light microscopy histology.

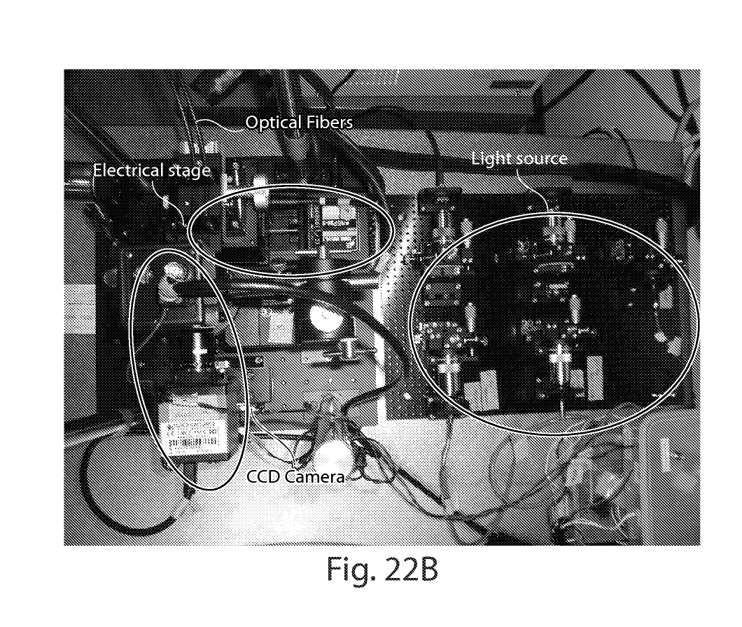

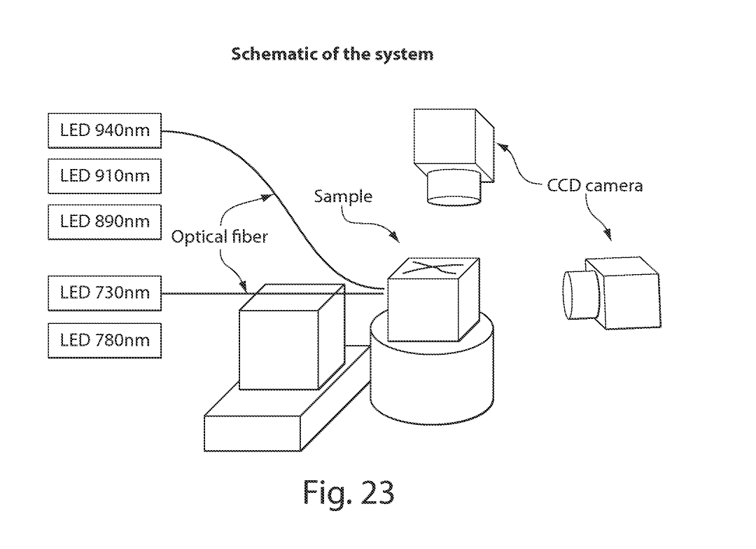

A system for detecting dental caries lesions and/or detecting bisphosphonate and/or pyrophosphonate bound to hydroxyapatite on a tooth is provided. See FIG. 22A showing the system including a computer adjacent to platform. The platform (top view in FIG. 22B) includes an electrical stage, optical fibers, a light source, and a charge coupled device (CCD) camera. A system and methods for detecting enhancement of remineralization of a tooth of a subject and/or detecting inhibition of a caries progression or loss of hydroxyapatite is shown in FIG. 23.

A tooth sample was placed on a stand or mount and was illuminated by the wavelengths produced by at least one optical fiber. The optical fibers directed electromagnetic light waves at specific wavelengths including 730 nm or 780 nm, or 890 nm, 910 nm, or 940 nm. A CCD camera then captured the image of the tooth and/or fluorescence emission from the tooth or fluorescent probe. The systems shown in FIGS. 22A and 22B and FIG. 23 were used in the following examples herein.

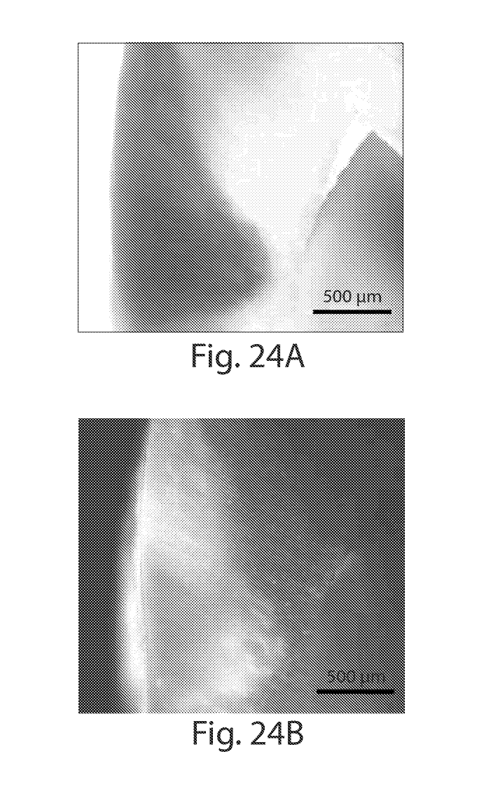

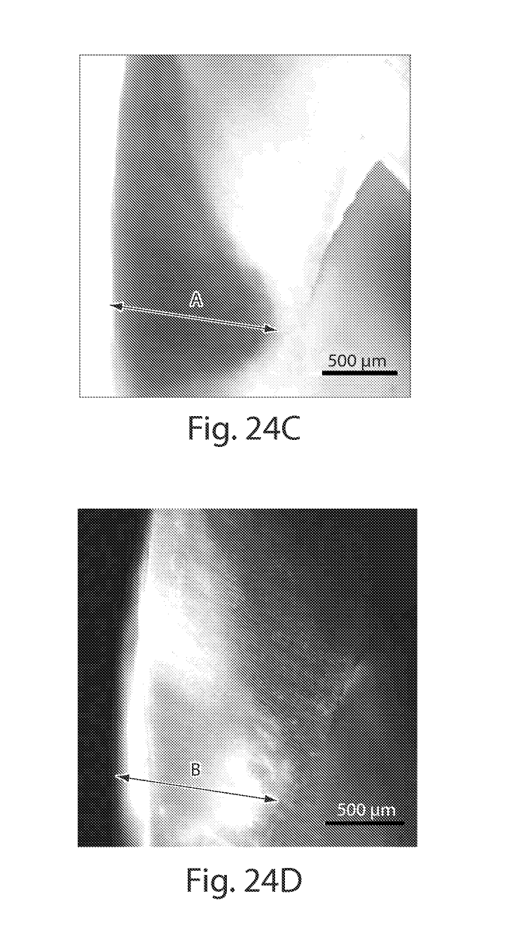

Thirty three extracted human teeth having caries were washed and visualized using a light microscope. FIG. 24A is a representative tooth image obtained by light microscope. OsteoSense 750, a bisphosphonate attached to a fluorescent cyanine dye, was then applied (1 μL of 1:100 dilution) to each tooth, and the teeth were illuminated with near infrared (NIR) light at 740 nm. A fluorescent signal from bisphosphonate-fluorescent dye probe bound to the caries was detected and photographed using a fluorescence microscope with an emission filter (780 nm). FIG. 24B is fluorescence microscope image of the representative tooth image in FIG. 24A.

Caries lesion depths were obtained from the light microscope images (defined as actual depth) and were compared to the depths determined by the fluorescent microscope. Percent infiltration of the fluorescent probe was calculated by dividing the depth of the caries determined by the fluorescence microscope by the depth of the caries determined by the light microscope. FIG. 24C and FIG. 24D are the set of representative tooth images obtained by light microscope and fluorescence microscope in FIG. 24A and FIG. 24B respectively, with the images showing the depth of the caries determined by the fluorescence microscope (B; FIG. 24D) and the depth of the caries determined by the light microscope (A; FIG. 24C).

TABLE. 13
Data showing individual extent of caries infiltration
for OsteoSense 750 compared to actual depth of caries

| Carious | Extent of caries |
|---|---|
| tooth | infiltration (%) |
| B | 96.37948 |
| C | 79.61783 |
| G | 88.4177 |
| H | 73.75126 |
| I | 92.85954 |
| J | 97.77629 |
| K | 103.9222 |
| M | 67.0466 |
| N | 121.522 |
| O | 69.84021 |
| P | 95.99854 |
| Q | 100.5699 |
| R | 84.8749 |
| S | 85.20505 |
| T | 93.86524 |
| U | 110.6269 |
| V | 71.51637 |
| W | 108.9507 |
| X | 93.86524 |
| Y | 111.465 |
| Z | 85.78917 |
| AA | 101.408 |
| BB | 59.64354 |
| CC | 93.30651 |
| EE | 99.00548 |
| FF | 92.42852 |
| GG | 102.2461 |
| HH | 106.197 |
| II | 109.6212 |
| JJ | 116.6611 |
| LL | 82.6908 |
| MM | 97.21757 |
| NN | 98.58897 |

Compiled data from teeth with lesions yielded an actual depth from light microscopy of 0.2 mm to 1.5 mm for the lesions. The extent of infiltration of the bisphosphonate-fluorescent dye probe was calculated and averaged 93.72±14.59% of the actual depth. Individual infiltration data for the teeth are shown in Table 13.

Further, the extent of infiltration was observed to be independent of depth of caries lesions (R=−0.0211; Pearson's correlation). Thus, the optical detection system herein using a bisphosphonate-fluorescent dye probe was found to be effective for measuring the depth of dental caries, and the data obtained were comparable to that obtained by using the standard light microscopy method.

### Example 18

### Inhibition of Hydroxyapatite Loss in Carious Teeth Using Bisphosphonate

Inhibition of loss of hydroxyapatite in carious teeth using bisphosphonate was analyzed by the light microscopy and fluorescence optical detection systems as described herein.

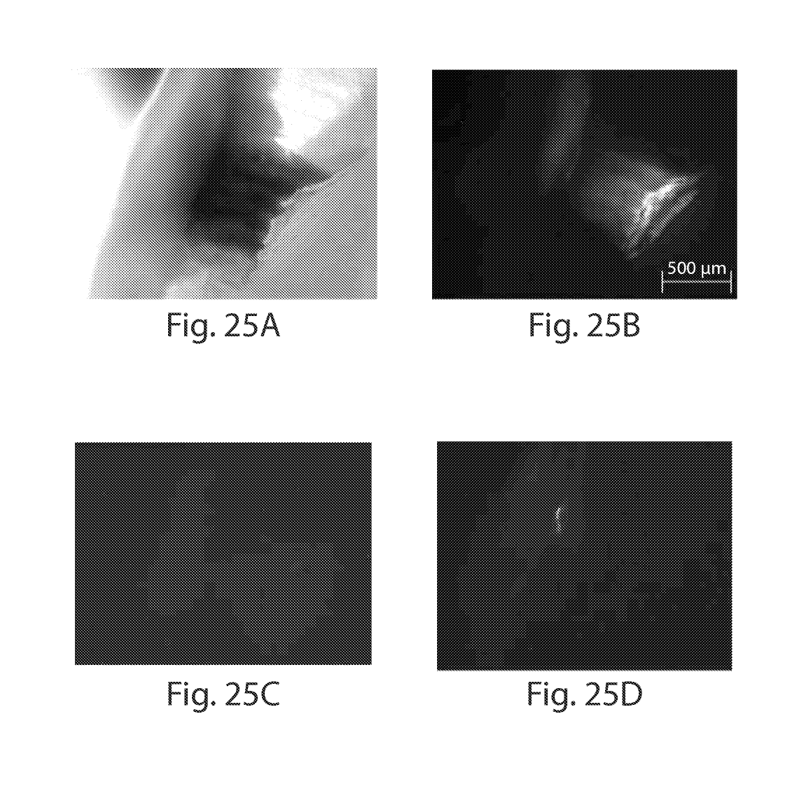

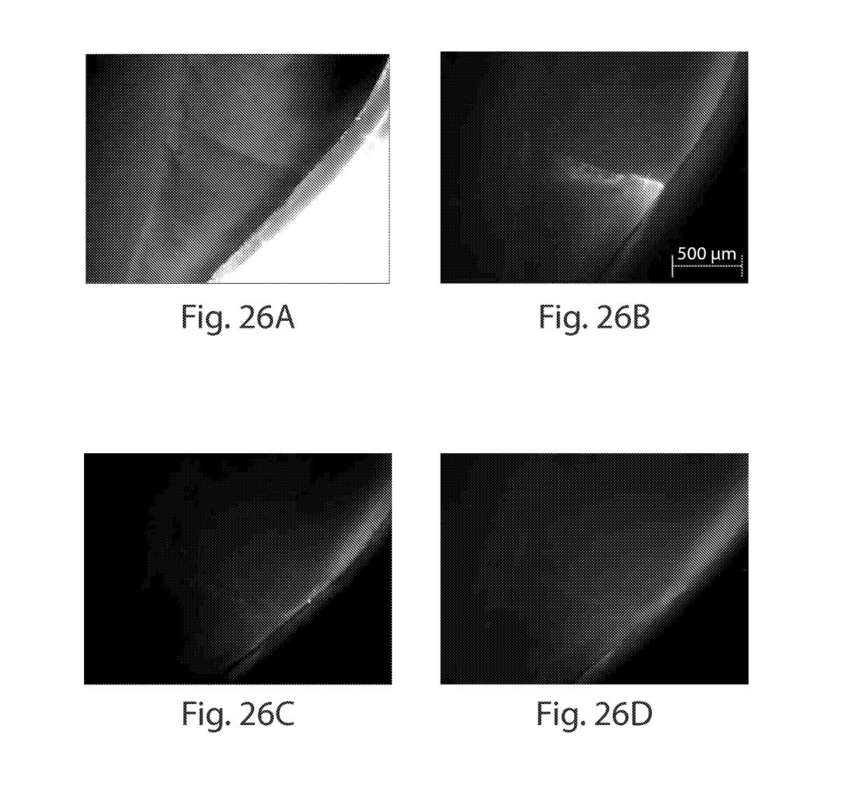

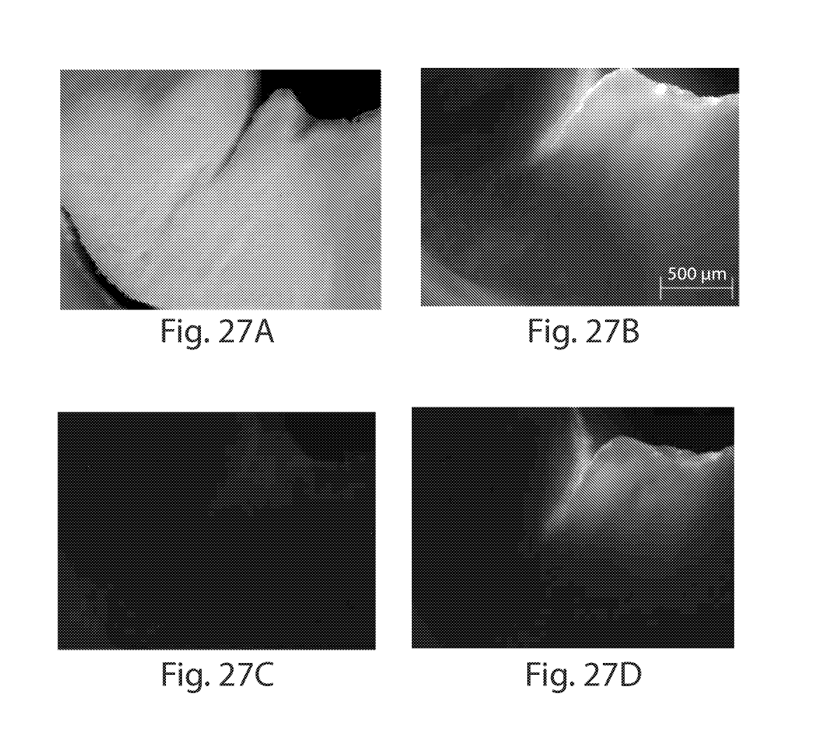

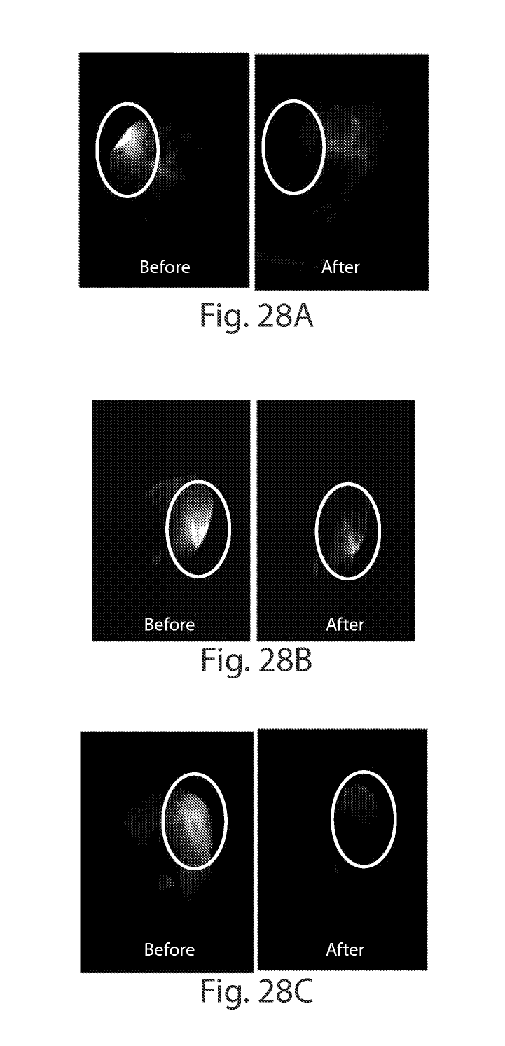

Three human extracted teeth were washed and examined histologically. Each tooth surface was determined to have a caries lesion. Teeth in FIG. 25A-FIG. 25D and FIG. 26A-FIG. 26D have interproximal caries and the tooth in FIG. 27A-D has occlusal caries. Further, the teeth were observed to differ in appearance, and depth and extent of caries.

Each tooth was then sectioned so as to expose the caries lesion, and contacted from the enamel surface with OsteoSense 750 (1 μL of 1:100 dilution from stock solution), a bisphosphonate attached to a fluorescent cyanine dye. FIGS. 25A, 26A and 27A show the caries in each tooth visualized by the light microscope. Teeth were illuminated with NIR light (740 nm) and a fluorescent signal from OsteoSense 750 bound to the caries was detected and photographed using a fluorescence microscope with an emission filter (780 nm) as shown in FIGS. 25B, 26B and 27B.

Comparison of the light microscope image (FIGS. 25A, 26A and 27A) and the fluorescence microscope image (FIGS. 25B, 26B and 27B) revealed that the bisphosphonate-fluorescent dye probe infiltrated at least 90% of the caries in each tooth. Percent infiltration of the fluorescent probe was calculated by dividing the depth of the caries determined by the fluorescence microscope (FIGS. 25B, 26B and 27B) by the depth of the caries determined by the light microscope (FIGS. 25A, 26A and 27A).

Each tooth was then treated with bisphosphonate by three times contacting the tooth from the enamel surface with OsteoSense 750 (1 μL of 1:30 dilution from stock solution). Tooth surfaces were rinsed with hydrogen peroxide (38%, Opalescence Boost; Ultradent Products Inc., South Jordan, Utah) to quench fluorescence from the fluorescent dye portion of OsteoSense 750.

Each tooth was then illuminated with NIR light and photographed using the fluorescence microscope. No discernible fluorescence emission was detected in the resulting fluorescence images. See FIGS. 25C, 26C and 27C. However, while the fluorescence from the cyanine dye portion of OsteoSense was no longer detected, the bisphosphonate portion of the OsteoSense was still bound to the hydroxyapatite in the tooth.

The teeth were contacted later with OsteoSense 750 (1 μL of 1:100 dilution from stock solution), illuminated with NIR light and photographed using a fluorescence microscope. FIGS. 25D, 26D and 27D show the resulting fluorescence images obtained from the fluorescence microscope. The resulting fluorescence images showed that little or no fluorescence emission was detected in the teeth. The insignificant amount of fluorescence emission detected in these images was observed to be located in shallower/proximal areas of the teeth rather than the deeper/distal areas of the teeth in which the initial fluorescence images had detected in FIGS. 25B and 25C, 26B and 26C and 27B and 27C.

FIGS. 25B, 25C and 25D, FIGS. 26B, 26C and 26D and FIGS. 27B, 27C and 27D show that OsteoSense 750 bonded to hydroxyapatite in the teeth and formed successive tooth layers proximal to the surface of the teeth of the teeth having erosions and caries. Further, the bisphosphonate-fluorescent dye probe caused the teeth having erosions and caries to have less porous hydroxyapatite. The reduced loss of hydroxyapatite and reduced porosity of the hydroxyapatite in teeth treated with OsteoSense 750 was confirmed by histology. These teeth no longer showed characteristics of porous surfaces associated with caries or erosions visualized in FIG. 25A, FIG. 25B, FIG. 26A, FIG. 26B, FIG. 27A and FIG. 27B. The tooth surfaces contacted with the OsteoSense 750 showed more uniform and stable hydroxyapatite during a period of days. These data show that the bisphosphonate resulted in restoration of the constitution of the dental hydroxyapatite, which would be useful in enhancing the remineralization of teeth.

Thus, application of OsteoSense 750, a bisphosphonate-fluorescent compound, resulted in restoration of the porous hydroxyapatite areas of the teeth having caries, and prevented further deterioration of the hydroxyapatite and/or the teeth.

### Example 19

### Bisphosphonate Inhibition of Caries in Enamel on Buccal Tooth Surfaces

Efficacy of a bisphosphonate compound to inhibit the progression of a caries lesion was assessed using a caries model test system, etching extracted tooth surfaces with acid.

Thirteen extracted human molars samples were collected and washed. Three sites on each buccal enamel surface (each a 2.0 mm diameter) were etched for 60 seconds with 38% phosphoric acid gel to demineralize the tooth surface, thereby producing simulated caries lesions. The demineralized area resulted in exposed hydroxyapatite material in the enamel.

The demineralized tooth surfaces were contacted with a bisphosphonate solution (OsteoSense 750, 1:30 dilution from the stock solution), a sodium fluoride foam (Denti-Foam 1.23% APF Fluoride Foam; Medicom Inc., Tonawanda, N.Y.), or not contacted as a negative control.

Tooth surfaces were rinsed with hydrogen peroxide (38%, Opalescence Boost; Ultradent Products Inc. South Jordan, Utah) to quench fluorescence from the applied OsteoSense 750. Inspection of the tooth surfaces contacted with hydrogen peroxide showed that the peroxide did not damage or otherwise affect the tooth surfaces.

A solution of OsteoSense 750 (1:100 dilution from stock solution) was applied to each tooth surface. The teeth were then illuminated with near infrared (NIR) light at 750 nm. Fluorescence from the OsteoSense 750 bound to the tooth surface was detected and photographed using a CCD camera with an emission filter (780 nm).

Extent of inhibition of loss of hydroxyapatite and/or coating of hydroxyapatite dental surface was evaluated by fluorescence values, such that less fluorescence indicates greater inhibition of hydroxyapatite breakdown and/or protection of dentals surfaces and reduced extent of binding of OsteoSense 750 to exposed hydroxyapatite in the enamel. Thus, the fluorescent probe acts as a reporter tool to monitor the extent of hydroxyapatite coating or protection.

The average fluorescence value and standard deviation for demineralized tooth surfaces contacted with bisphosphonate was 22.1±8.6, less than that of surfaces contacted with sodium fluoride (29.9±9.5) and of the negative control (34.6±8.611.9). These data were statistically significant (p<0.01, ANOVA and Fisher's PLSD). Thus bisphosphonate was here shown to be efficacious for protecting enamel surfaces having exposed hydroxyapatite and inhibiting further loss of hydroxyapatite. The extent of coating and/or protection of enamel hydroxyapatite were greater than results from the conventional technique of applying fluoride foam only. The difference in fluorescence for bisphosphonate-treated teeth compared to the untreated teeth, 12.0, is about three times greater than that of the fluoride treated tooth (4.2).

### Example 20

### Bisphosphonate Inhibition of Enamel Caries on Interproximal Tooth Surfaces

Bisphosphonate inhibition of caries lesions progression assessed using interproximal enamel caries lesions in 16 extracted human teeth.

To obtain a pre-treatment fluorescence measurement, a solution of OsteoSense 750 (1:100 dilution from the stock solution) was then applied to each interproximal caries lesion.

A thin layer of transparent nail varnish was applied to the area surrounding each interproximal early stage caries. Each tooth was then sectioned to expose the caries lesion and a thin layer of transparent nail varnish was applied to the sectioned surface.

The teeth sections were illuminated with near infrared (NIR) light at 750 nm. A fluorescent signal from OsteoSense 750 dye probe bound to the caries was detected and photographed using a charge coupled device (CCD) camera with an emission filter (780 nm) Thus, a caries lesion in the interproximal enamel surface was observed. The average fluorescence value and standard deviation for the interproximal caries lesions was 102.6±23.9.

A solution of OsteoSense 750 (1:30 dilution from a 1 mg/mL stock solution) was applied to each interproximal caries lesion to coat and/or protect the demineralized teeth. Each tooth was then rinsed with water. Applying the OsteoSense 750 and rinsing the tooth was performed at least three times. The teeth were then contacted with hydrogen peroxide (38%, Opalescence Boost; Ultradent Products Inc., South Jordan, Utah) to quench the fluorescence from the fluorescent dye probe portion of the OsteoSense 750, which was bound to the caries lesions.

To determine the extent that bisphosphonate inhibits caries progression, a solution of OsteoSense 750 (1:100 dilution from stock solution) was applied to each interproximal caries lesion, and teeth were illuminated with near infrared (NIR) light at 750 nm. A fluorescent signal from OsteoSense 750 dye probe bound to the caries was detected and photographed using a CCD camera with an emission filter (780 nm). The average fluorescence value and standard deviation for the interproximal caries was 38.85±14.9.

These data show that reduction in fluorescence value compared to the fluorescence signal of the interproximal caries prior to the application of OsteoSense 750 (102.6±23.9) was 62%. Similar to example 19 herein, the lower fluorescence value indicates an extent that bisphosphonate prevented further hydroxyapatite loss from the demineralized tooth surface and coated the tooth surface. FIG. 26A-FIG. 26D show representative tooth fluorescence data indicating that lower fluorescence emission was observed after the bisphosphonate treatment compared to the fluorescence emission prior to the bisphosphonate treatment. These data show that the bisphosphonate treatment coated and/or protected the interproximal tooth surface. Further, the area having the caries lesion was observed to be less porous with less exposed hydroxyapatite as a result of the bisphosphonate treatment. Thus, the bisphosphonate treatment coated the interproximal surfaces of the teeth including the hydroxyapatite surfaces and inhibited hydroxyapatite breakdown and caries progression.

### Example 21

### Synthesis of Bisphosphonate Compounds

Bisphosphonate compounds are synthesized to determine their ability to bind to hydroxyapatite in teeth, enhance remineralization and to coat teeth. The compounds are made using organic chemistry techniques and the structures of the compounds are confirmed and analyzed using nuclear magnetic resonance (NMR). The compounds synthesized are represented by the structural formula below

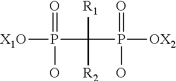

In the above structural formula, R₁ and R₉ are independently synthesized from a (C₁-C₁₈)alkyl, a (C₁-C₁₈)heteroalkyl, a (C₁-C₁₈)alkoxy, a (C₁-C₁₈)heteroalkyl, a (C₆-C₁₀)aryl, a (C₁-C₉)heteroaryl, and a (C₆-C₁₀)aryl(C₁-C₆)alkyl. For example R₁, and R₂ include functional groups including aliphatic groups, olefin groups (monounsaturated and polyunsaturated), and heteroatom groups (e.g., oxygen, nitrogen, and fluoride). In the above structural formula, X₁ and X₂ are independently synthesized from: (C₆-C₁₀)aryl, (C₁-C₉)heteroaryl, (C₆-C₁₀)aryl(C₁-C₆)alkyl, (C₁-C₉)heteroaryl(C₁-C₆)alkyl, (C₆-C₁₀)aryl(C₆-C₁₀)aryl, (C₁-C₉)heteroaryl(C₆-C₁₀)aryl, (C₆-C₁₀)aryl(C₁-C₉)heteroaryl, (C₁-C₉)heteroaryl(C₁-C₉)heteroaryl, (C₆-C₁₀)aryloxy(C₆-C₁₀)aryl, (C₁-C₉)heteroaryloxy(C₆-C₁₀)aryl, (C₆-C₁₀)aryloxy(C₁-C₉)heteroaryl, (C₁-C₉)heteroaryloxy(C₁-C₉)heteroaryl, (C₆-C₁₀)aryloxy(C₁-C₆)alkyl, (C₁-C₉)heteroaryloxy(C₁-C₆)alkyl, (C₆-C₁₀)aryl(C₁-C₆)alkyl(C₆-C₁₀)aryl, (C₁-C₉)heteroaryl(C₁-C₆)alkyl(C₆-C₁₀)aryl, (C₆-C₁₀)aryl(C₁-C₆)alkyl(C₁-C₉)heteroaryl, (C₁-C₉)heteroaryl(C₁-C₆)alkyl(C₁-C₉)heteroaryl, (C₆-C₁₀)aryl(C₁-C₆)alkoxy(C₆-C₁₀)aryl, (C₁-C₉)heteroaryl(C₁-C₆)alkoxy(C₆-C₁₀)aryl, (C₆-C₁₀)aryl(C₁-C₆)alkoxy(C₁-C₉)heteroaryl, (C₁-C₉)heteroaryl(C₁-C₆)alkoxy(C₁-C₉)heteroaryl, (C₆-C₁₀)aryloxy(C₁-C₆)alkyl(C₆-C₁₀)aryl, (C₁-C₉)heteroaryloxy(C₁-C₆)alkyl(C₆-C₁₀)aryl, (C₆-C₁₀)aryloxy(C₁-C₆)alkyl(C₁-C₉)heteroaryl, (C₁-C₉)heteroaryloxy(C₁-C₆)alkyl(C₁-C₉)heteroaryl, (C₆-C₁₀)aryl(C₆-C₁₀)aryl(C₁-C₆)alkyl, (C₁-C₉)heteroaryl(C₆-C₁₀)aryl(C₁-C₆)alkyl, (C₆-C₁₀)aryl(C₁-C₉)heteroaryl(C₁-C₆)alkyl, (C₁-C₉)heteroaryl(C₁-C₉)heteroaryl(C₁-C₆)alkyl, (C₆-C₁₀)aryl(C₁-C₆)alkoxy(C₁-C₆)alkyl, or (C₁-C₉)heteroaryl(C₁-C₆)alkoxy(C₁-C₆)alkyl, such that, independently, each of the ring carbon atoms of the (C₆-C₁₀)aryl and (C₁-C₉)heteroaryl moieties that is capable of forming an additional bond by a group such as fluoro, chloro, bromo, (C₁-C₆)alkyl, (C₁-C₆)alkoxy, and perfluoro(C₁-C₃)alkyl, and perfluoro(C₁-C₃)alkoxy.

The compounds are also formulated with various substances including polymers, oils, resins, fillers, solvents, and buffers to modulate the rate of removal of the compound in the oral cavity. For example compounds were formulated with different percentages of bioresorable materials.

The compounds are compounded as dental formulations for administration to the teeth and/or oral cavity including variations in the pH, solubility, viscosity, and concentration. The compounds are mixed thoroughly and placed in containers prior to use.

### Example 22

### Bisphosphonate Compounds Inhibit Hydroxyapatite Breakdown in Teeth In Vivo

The bisphosphonate compounds are topically applied to demineralized teeth ex vivo and in vivo for a specific period of time (e.g., minutes, hours, and days). The teeth are analyzed for inhibition of hydroxyapatite loss as a function of time. The bisphosphonate compounds are found to protect the teeth from further hydroxyapatite loss and to enhance development of organized layers and patterns of hydroxyapatite material in the teeth when the tooth is remineralized. Radiological examination and histological examination of the teeth show that the crystalline structure of the teeth is restored or drastically improved after being contacted with the bisphosphonate compounds and that the hydroxyapatite is less porous. Further addition of polymers and resins is observed to affect the rate and extent of the bisphosphonate binding to the dental hydroxyapatite, and thus the rate and degree that bisphosphonate makes the hydroxyapatite less porous. Contacting to teeth with a bisphosphonate compound for a greater period of time is found to result in greater protection of teeth, for example, the hydroxyapatite is less likely to breakdown or erode from the tooth. Thus, the bisphosphonate compounds are effective therapeutic agents ex vivo and in vivo.

### Example 23

### Pyrophosphonate Compounds

Pyrophosphonate compounds are synthesized to determine their ability to enhance remineralization and to inhibit loss of hydroxyapatite from teeth compared to bisphosphonate compounds. The pyrophosphonate compounds are synthesized using organic chemistry techniques, and the structures of the compounds are confirmed and analyzed using NMR. The compounds synthesized are represented by the structural formula below.

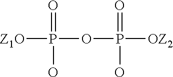

In the above structural formula, Z₁ and Z₂ are independently synthesized from: (C₆-C₁₀)aryl, (C₁-C₉)heteroaryl, (C₆-C₁₀)aryl(C₁-C₆)alkyl, (C₁-C₉)heteroaryl(C₁-C₆)alkyl, (C₆-C₁₀)aryl(C₆-C₁₀)aryl, (C₁-C₉)heteroaryl(C₆-C₁₀)aryl, (C₆-C₁₀)aryl(C₁-C₉)heteroaryl, (C₁-C₉)heteroaryl(C₁-C₉)heteroaryl, (C₆-C₁₀)aryloxy(C₆-C₁₀)aryl, (C₁-C₉)heteroaryloxy(C₆-C₁₀)aryl, (C₆-C₁₀)aryloxy(C₁-C₉)heteroaryl, (C₁-C₉)heteroaryloxy(C₁-C₉)heteroaryl, (C₆-C₁₀)aryloxy(C₁-C₆)alkyl, (C₁-C₉)heteroaryloxy(C₁-C₆)alkyl, (C₆-C₁₀)aryl(C₁-C₆)alkyl(C₆-C₁₀)aryl, (C₁-C₉)heteroaryl(C₁-C₆)alkyl(C₆-C₁₀)aryl, (C₆-C₁₀)aryl(C₁-C₆)alkyl(C₁-C₉)heteroaryl, (C₁-C₉)heteroaryl(C₁-C₆)alkyl(C₁-C₉)heteroaryl, (C₆-C₁₀)aryl(C₁-C₆)alkoxy(C₆-C₁₀)aryl, (C₁-C₉)heteroaryl(C₁-C₆)alkoxy(C₆-C₁₀)aryl, (C₆-C₁₀)aryl(C₁-C₆)alkoxy(C₁-C₉)heteroaryl, (C₁-C₉)heteroaryl(C₁-C₆)alkoxy(C₁-C₉)heteroaryl, (C₆-C₁₀)aryloxy(C₁-C₆)alkyl(C₆-C₁₀)aryl, (C₁-C₉)heteroaryloxy(C₁-C₆)alkyl(C₆-C₁₀)aryl, (C₆-C₁₀)aryloxy(C₁-C₆)alkyl(C₁-C₉)heteroaryl, (C₁-C₉)heteroaryloxy(C₁-C₆)alkyl(C₁-C₉)heteroaryl, (C₆-C₁₀)aryl(C₆-C₁₀)aryl(C₁-C₆)alkyl, (C₁-C₉)heteroaryl(C₆-C₁₀)aryl(C₁-C₆)alkyl, (C₆-C₁₀)aryl(C₁-C₉)heteroaryl(C₁-C₆)alkyl, (C₁-C₉)heteroaryl(C₁-C₉)heteroaryl(C₁-C₆)alkyl, (C₆-C₁₀)aryl(C₁-C₆)alkoxy(C₁-C₆)alkyl, or (C₁-C₉)heteroaryl(C₁-C₆)alkoxy(C₁-C₆)alkyl, such that, independently, each of the ring carbon atoms of the (C₆-C₁₀)aryl and (C₁-C₉)heteroaryl moieties that is capable of forming an additional bond by a group such as fluoro, chloro, bromo, (C₁-C₆)alkyl, (C₁-C₆)alkoxy, and perfluoro(C₁-C₃)alkyl, and perfluoro(C₁-C₃)alkoxy.

The compounds are also formulated with various relative concentrations of compound compared to substances including polymers, resins and emulsifiers. Compounds are mixed and stored in containers.

The pyrophosphonate compounds are applied to demineralized teeth or eroded teeth in vivo, and analyzed as a function of time. The pyrophosphonate compounds applied to teeth are observed to inhibit porosity of the hydroxyapatite and the results are comparable to the results observed for bisphosphonate compounds. Thus, the pyrophosphate compounds bind to and protect teeth ex vivo and in vivo.

### Example 24

### Delivery Devices for Bisphosphonate and/or Pyrophosphonate

Devices for administration of bisphosphonates and/or pyrophosphonates are manufactured and tested to identify effective vehicles for delivering the therapeutic agents. Bisphosphonate compounds and/or pyrophosphonate compounds that are determined to effectively inhibit hydroxyapatite loss in teeth when topically applied are selected and used to prepare oral devices. The compounds are added to substrates and/or materials including sponges, membranes, and films. The compounds are formulated in amounts that are non-toxic to the subject and with pharmaceutically acceptable diluents, buffers, solvents and agents.

Oral devices are sterilized, lyophilized, incubator dried, air dried, or centrifuged, such so that the final product is effectively dry and stable, and/or are constructed with synthetic adhesives for maintaining contact with a tooth and/or oral cavity after placement.

The oral devices are tested for ability to affect teeth and bones upon direct contact to a tooth or mouth surface. For example, the oral devices with adhesives fixedly attach to the buccal surface or lingual surface of a demineralized tooth, the gingiva adjacent to the root of a demineralized tooth, or on a tooth adjacent to a demineralized tooth. The inhibition of hydroxyapatite loss and increased hydroxyapatite strength are determined over a period of minutes, hours and days. The devices are removed from the tooth or oral cavity by the dentist or patient after a prescribed period of time, or passively losing contact with the tooth or the oral cavity. The oral devices manufactured using films are tested by placement on the tongue and salivary fluids dissolve the agent for delivery to the entire oral cavity.

The oral devices including bisphosphonates and/or pyrophosphonates are shown to inhibit loss of hydroxyapatite, to prevent hydroxyapatite from becoming porous, and also to enhance the effects of remineralizing agents applied to teeth.

### Example 25

### Detection of Caries Using NIR Fluorescent Bisphosphonates Having Wavelengths Distinct from Enamel and Dentin Autofluorescence Wavelengths

An optical system for early caries detection was developed using synthesized NIR fluorescence bisphosphonate derivative probes. No clinical application is currently available to detect early interproximal caries because of low sensitivity for incipient caries. Examples herein used methods and compositions that combined the technology of NIR transillumination and NIR fluorescence bisphosphonate probes to detect early stage dental caries.

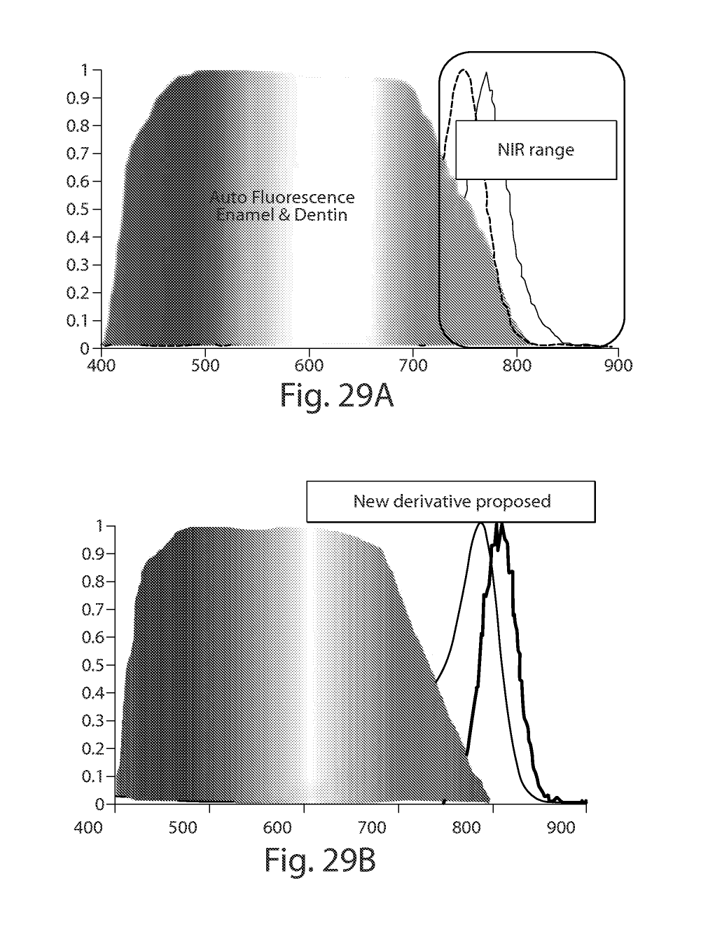

Early stage dental caries lesions were diagnosed in Examples herein using OsteoSense750 which has an excitation wavelength of 730 nm and an emission wavelength of 750 nm (See FIGS. 24A-B, FIGS. 25A-D, and FIG. 26A-D). Analysis of a probe OsteoSense 750 shows that the excitation and emission wavelengths overlap with the autofluorescence wavelengths of dental enamel and dentin (FIG. 29A).

A carious tooth was contacted with a different probe, OsteoSense800 (PerkinElmer Inc.; Waltham Mass.; product number NEV11105) to determine whether contacting caries lesions with this probe having excitation and emission wavelengths higher than tooth enamel and dentin autofluorescence would result in a clearer caries image with less light scatter (FIG. 29B). OsteoSense 800 has an excitation wavelength of 780 nm and an emission wavelength of 800 nm which is 50 nm greater in excitation and emission wavelength than OsteoSense 750.

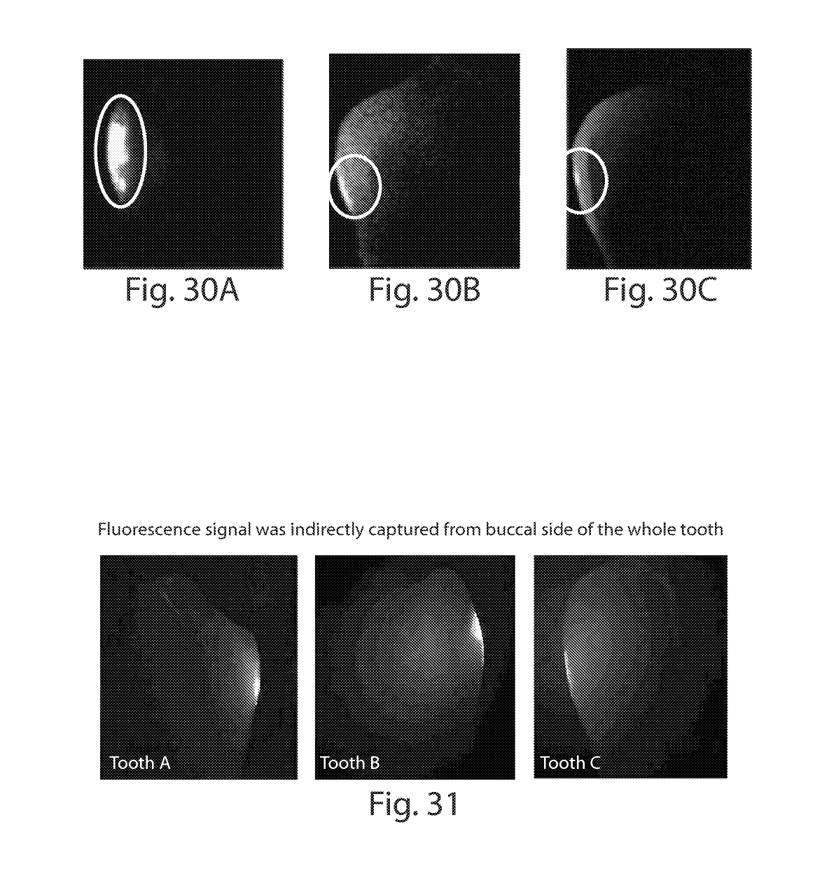

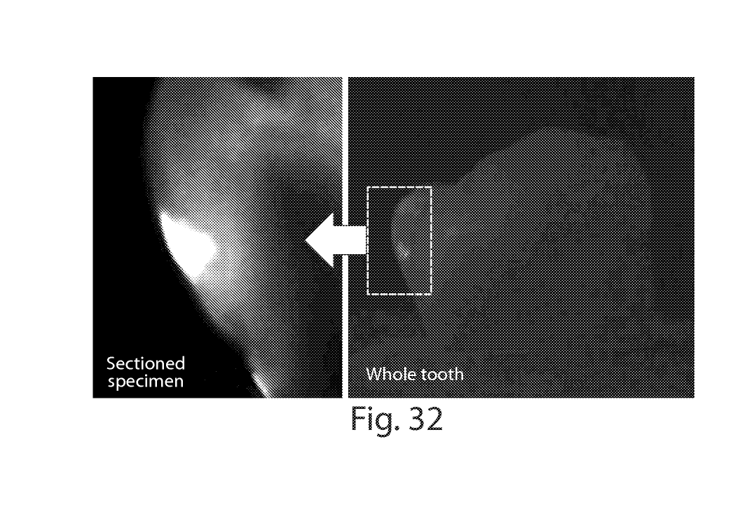

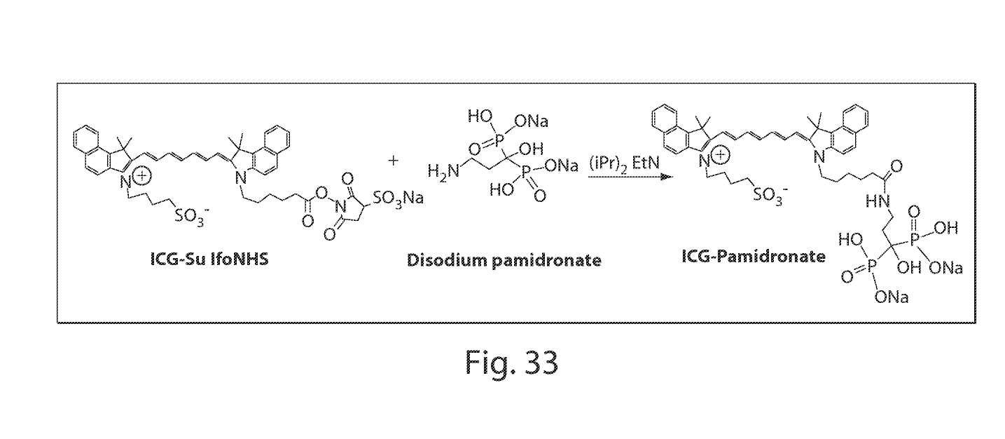

Teeth having enamel caries were contacted with either OsteoSense 750 or OsteoSense 800, illuminated with the corresponding excitation wavelengths and were imaged from the buccal surface of the tooth using a fluorescence camera. Data (FIGS. 30A-C and FIG. 31) show improved resolution of fluorescence caries images for teeth contacted with OsteoSense 800 compared to fluorescence caries images for teeth contacted with OsteoSense 750 (compare FIGS. 30A-C and FIG. 31).

These data show that a method of selection of an NIR fluorescence bisphosphonate derivative with an optimized profile of excitation and emission wavelengths distinct from the autofluorescence range of enamel and dentin improved visualization and identification of caries lesions.

### Example 26

### Synthesis of NIR Fluorescent ICG-Bisphosphonates and ICG-Pyrophosphonates

NIR bisphosphonate and ICG-pyrophosphonates were synthesized and characterized to target exposed hydroxyapatite and to have a fluorescence excitation wavelength greater than about 810 nm and emission wavelength greater than about 830 nm, which does not overlap with tooth enamel autofluorescence (about 400 nm to about 800 nm), to improve optical imaging characteristics of the probe bound to a caries lesion.

NIR dyes in addition to those described above having excitation/emission wavelengths greater than about 800 nm or 80 nm are chemically attached to the bisphosphonate probes. The NIR dyes initially selected were derivatives of indocyanine green (ICG), ICG-N-hydroxysulfosuccinimide ester (Osu) which has an excitation wavelength of and excitation wavelength greater than wavelengths of tooth autofluorescence, and ICG-PF which has an excitation wavelength of about 810 nm and excitation wavelength of about 830 nm. Thus each of the NIR dyes used has a higher excitation and emission wavelengths than the autofluorescence wavelengths of enamel and dentin. ICG-OSu and ICG-PF are indocyanine green dyes in the chemical structures of which contain activated N-HydroxySuccinimide (NHS) esters, as points of attachment and conjugation to bisphosphonates and pyrophosphonates.

The near infrared ICG-bisphosphonate compounds synthesized are for example ICG-pamidronate derivatives. The derivatives are synthesized using standard chemical conjugation methods that use the structural characteristics of the ICGs (e.g., NHS esters). See Christoffersen J. et al. 1991 J Dent Res 70: 123-126; and Budz J. A. et al. 1988 J Dent Res 67: 1493-98). The ICG precursors, for example ICG-OSu and ICG-PF, are reacted with the primary amine of pamidronate in the presence of the base catalyst 3-hydroxypropionitrile to yield an ICG-pamidronate (FIG. 9). Additional reactions of this same chemical type are performed with additional nitrogen containing bisphosphonates such as neridronate, olpadronate, alendronate, ibandronate, risedronate, and zoledronate to yield additional probes and remineralizations agents, in the class of ICG-substituted bisphosphonates. Additional reactions are also performed with pyrophosphonates to form ICG-substituted pyrophosphonates.

The ICG-bisphosphonate and ICG-pyrophosphonates compounds/products are purified and isolated using preparative high pressure liquid chromatography (HPLC). The HPLC purified ICG-compounds are subjected to complete chemical characterization using high-resolution mass spectroscopy (HRMS) and proton nuclear magnetic resonance (¹H-NMR) analyses. Purity and authentication of the product and structure are also determined. Further fluorescence excitation/emission profiles of the each ICG-bisphosphonate and ICG-pyrophosphonates compounds are measured and determined by analytical methods such as fluorescence emission spectroscopy. The sensitivity and extinction coefficients are calculated using as standards the excitation/emission profiles of known concentration solutions. The ICG-bisphosphonate and ICG-pyrophosphonates compounds showing the best NIR-optical imaging characteristics (e.g., high sensitivity and low autofluorescence background) are selected for use in further ex vivo and in vivo analyses of caries detection and remineralization.

### Example 27

### Low Toxicity of ICG-Bisphosphonate Conjugates/Products

Bisphosphonate-related osteonecrosis of the jaw is a documented complication related to oral bisphosphonate therapy for osteoporosis (Drake M. T. et al. September 2008 Mayo Clinic Proceedings vol. 83(9): 1032-1045; Siris E. S. et al. August 2006 Mayo Clin Proc. 81(8): 1013-1022; and Deepak T. et al. August 2006 Mayo Clin Proc. 81(8): 1100-1103). To determine whether topical application of bisphosphonate derivatives induces deterioration and/or weakening of the bone, animal subjects are treated with the selected synthesized ICG-substituted bisphosphonate and pyrophosphonate products described herein.

Sets of subjects are treated using different routes of administration (e.g., topical application, soaking, injection), application frequency (e.g., one, two, four, eight, sixteen treatments per year) and doses. For example an OsteoSense750 solution (20 nmol/1.50 μl) is diluted to a final concentration of 1.33×10⁻³ nmol/μl (1.46×10⁻⁶ mg/μl). An aliquot (1 μl/tooth) of the OsteoSense 750 solution is used for a significant treatment area of a mouth (e.g., the set of entire group of teeth in a mouth or four teeth, or twelve teeth).

Data are predicted to show no toxicity or necrosis for subjects treated with any of the number of treatments, such as six, eight, or ten treatments per year. Extremely low concentrations of bisphosphonate or pyrophosphonate were administered for diagnostic use, and the total exposure per patient was observed to be orders of magnitude lower than known toxic dose. Further analysis is performed to optimize the exact amount of bisphosphonates or pyrophosphonates necessary to safe detection and remineralization of teeth, the administration method, and appropriate storage methods. Additional in vivo and ex vivo data are obtained to determine parameters and conditions for effective and safe administration of the compounds.

### Example 28

### Analysis of Accuracy of Caries Detection Using Ex Vivo NIR Fluorescence Imaging

Examples herein assessed efficacy of a caries detection system using OsteoSense750 by measuring the sensitivity, specificity, positive predictive value, and negative predictive value (NPV) of the OsteoSense 750 system in comparison with conventional X-radiography. Tables 3 and 12; See also Gil M. et al. Assessment of incipient interproximal caries diagnosis with new optical technology, IADR/AADR/CADR 88th General Session and Exhibition abstract #2822; and Khorashadi S. et al. Detection of incipient interproximal legions using dye enhanced fluorescence, IADR/AADR/CADR 88th General Session and Exhibition abstract #2821). The mean sensitivity of OsteoSense 750 was 89.3% and of X-ray methods was 32.0% (p<0.0001). The mean NPV of OsteoSense 750 was 77.5% and the NPV of X-ray methods 34.8% (p<0.0004). The fluorescent data and X-ray data were compared to the true depth of the caries using histological methods including sectioning the teeth and then using micro single-photon emission computed tomographic (Micro-SPECT) imaging and the Dignopent imaging unit (Kayo Inc., Biberach/Riss, Germany). A non-destructive sampling method was investigated that does not require extensive sample preparation and sectioning of a tooth in order to facilitate rapid analysis of the caries lesions. This method would allow for accurate measurement of sensitivity, specificity, positive predictive value, and negative predictive value of the additional ICG-bisphosphonates and ICG-pyrophosphonates.

The Xradia MicroXCT-200 microscope, a high resolution three-dimensional (3D) X-ray microscope with switchable scintillator-objective lenses, was selected for use, which produces high quality tomographic images through the entire tooth (Xradia Inc.; Pleasanton, Calif.). The Xradia MicroXCT-200 microscope yields high spatial resolution down to one micrometer. After reconstruction of the digital data, these images are studied using Xradia 3D viewing software that analyzes the tooth in at least three different planes and in three dimensions without compromising image integrity. Conventional two-dimensional based systems require serial cross sectioning that can result in damage and loss of data and provide information in only one plane.

Early caries lesions (observed as white spots on the tooth) were visualized using the Xradia MicroXCT 3D X-ray microscope and were then sectioned and visualized using a light microscope (FIGS. 34A-C and FIGS. 35A-C). Tooth samples analyzed using the light microscope showed a demineralized tooth area (FIG. 34B). A fluorescence signal image (FIG. 34) of the tooth administered OsteoSense 750 show the caries lesion on the same part of the tooth. Data show that the Xradia microscope identified the caries lesion in the same area of the tooth (FIG. 34C) identified by the light microscope and fluorescence emission (FIGS. 34A-B).

Further, the Xradia microscope identified small caries lesions that normally would not be identifiable without sectioning the tooth. In another representative tooth, a very small caries lesion (about 100 μm in size) was identified by the Xradia, and the probe and the presence of the caries was confirmed by the histological examination (FIGS. 35A-C). Thus, the Xradia microscope identified shallow caries lesions (less than 500 μm in size/depth) and produced a high resolution (to 1.0 micrometer) microstructural analysis of a tooth sample.

The Xradia MicroXCT-200 microscope was effective for identifying caries and does not require sectioning of a tooth in order to identify a caries lesion. Thus, a method using Xradia MicroXCT-200 microscope was used as a basis of comparison to identify the presence of a caries in a tooth and comparing the sensitivity, specificity, positive predictive value, and negative predictive value of X-ray methods and the fluorescent detection system described herein.

### Example 29

### Statistical Analysis of Bisphosphonate or Pyrophosphonate Compounds Using a Xradia Microscope

Data are obtained using the MicroXCT 3D X-ray microscope to determine whether the optical detection system described herein using bisphosphonates and/or pyrophosphonates accurately detects caries depth and location comparable to X-ray methods, which are currently the gold standard approach for caries detection.

A drawing of an ex vivo imaging system used to detect caries in a typodont is shown in FIG. 37. A typodont including teeth and gingiva, is illuminated at or through (transillumination) with wavelengths of light using a light source. A B/W CCD camera with cut filter of visible light for detecting emission wavelengths of light and/or an image of the tooth is connected to a battery box. The capture board digitizes an incoming stream of video signal and data (e.g., analogue) and is connected to the laptop computer or personal computer (PC) using an implementers forum-certified universal serial bus (I/F USB) hub or connector.

Extracted human permanent premolars and molars without restorations and significantly cavitated lesions are obtained, collected and placed in the typodont shown in FIG. 37. The teeth are kept in distilled water from the time of extraction. Approximately 30 teeth with sound surfaces and 70 teeth with white spot lesions are used for examination for a total of 100 to 200 interproximal surfaces.

Prior to examination teeth are cleaned to remove external staining, calculus, and plaque with a standard cleaning procedure using dental scrapers and devices. The teeth are contacted with NIR ICG-bisphosphonate or ICG-pyrophosphonate compounds (about 1 μl) in an interproximal area of the tooth with a microbrush, and the area is rinsed with water (FIG. 38) by a method that corresponds to a clinical setting, in which patients expectorate the rinse water, or the rinse is suctioned by the dentist resulting in a low level of ingestion of the compound by the patient.

For evaluation of data, a blind panel of at least five dentists is used. The dentists are not informed as to either the presence or location of caries lesions. The light source for excitation in the system is a class 3R laser with restricted beam viewing, which is accepted as safe for clinical use. Dentists scan each interproximal area with a pen type illumination light source such as a class 3R laser from the occlusal surface and a handheld CCD camera is placed on the buccal side. Real-time images with tooth anatomy and fluorescence signal are shown on the computer display and are recorded. Illumination of teeth is directed from the occlusal surface, lingual surface or buccal surface. Illumination is performed using at least two wavelengths (940 nm for tooth anatomy and 810 nm for fluorescence excitation; 30 illuminations per second). The MPE (maximum permissible exposure) using a class 3R laser is not exceeded, and the parameters are chosen so that even if exceeded only a low risk of injury to the teeth results. The visible continuous lasers in Class 3R are limited to about five milliwatts (mW). Alternatively, a 2.5 mW light emitting diode (LED) light source is used in conjunction with safety goggles.

A representative image of a tooth contacted with OsteoSense 800 and imaged using the system described in FIG. 37 is shown in FIGS. 39A-C. Dentists use the camera to take indirect images of fluorescence signal to see the depth of caries lesion. Real time images are automatically recorded. The panel of dentists analyzes the images of the teeth in laboratory, an office, or a dark room. Fluorescence signal from the bound probes expires after about 24 hours, such that dentists view the bound probe at approximately the same fluorescence signal intensity in this period.

A conventional bitewing radiograph of each tooth is obtained by imaging the tooth perpendicular to the interproximal area. A CMOS (Complementary metal-oxide-semiconductor) sensor is used and the X-ray image is obtained (70 kvp, 7 mA, 5 pulses).

Dentists identify and record the images as one of the following: sound; white-spot lesions (less than one-half the depth of the enamel); advanced white-spot lesions (greater than one-half the depth of enamel); or caries lesions beyond/on the dentin-enamel junction. Each dentist makes a real-time diagnosis and reviews recorded video to confirm the diagnosis.

The Xradia MicroXCT-200 microscope is used as a standard method for comparison purposes to determine a true size and/or a depth of each lesion. The tooth is mounted on a rod, placed into a stand and scanned using the Xradia MicroXCT-200. To standardize data, tooth samples are scanned using the same filter, magnification, exposure time, kV, voltage and slice number. Following reconstruction of the digital data, the images are analyzed using the Xradia viewing software. The depth of lesions is assessed at the deepest area, and categorized as one: sound; white-spot lesions (less than one-half the depth of the enamel); advanced white-spot lesions (greater than one-half the depth of enamel); or caries lesion beyond/on the dentin-enamel junction. A representative image of a caries tooth scanned and imaged by the Xradia MicroXCT-200 microscope is shown in FIGS. 40A-D.

Following evaluation by Xradia MicroXCT-200, teeth are sectioned to examine size and depth of the caries. Each tooth is sectioned, ground and polished using Buehler Precision saw (IsoMet® 1000 Precision Saw) and Buehler Polisher (Vector Powerhead AutoMet® 2000) from the buccal and lingual surfaces into sections that are approximately 1 mm in thickness, to isolate each interproximal site. The lesion size and depth is measured using a fluorescence microscope. A non-resonant scanner and a resonant scanner are incorporated in one unit, and fluorescence imaging is conducted simultaneously without a separate laser unit for photo activation. The resonant scanner captures the images at high speed, and rapid changes after photo activation are recorded. The depth of lesion and depth of infiltration of a new NIR ICG-bisphosphonates and ICG-pyrophosphonates described herein are compared, and the ratios of caries infiltration for each site are calculated.

The results of the evaluators' assessments of each specimen are then compared to results obtained using the Xradia MicroXCT-200 imaging device. The overall sensitivity, specificity, positive predictive value, and negative predictive value for each detection method are calculated. Standard errors are estimated using bootstrap analysis. Statistical analyses are performed using the SAS version 9.1 statistical package (SAS Institute, NC, USA). The intra-examiner reliability is calculated for each dentist using Cohen's kappa. The inter-examiner reliability for each detection method is calculated using Fleiss' kappa. Data show that the optical detection system described herein using ICG-bisphosphonates and ICG-pyrophosphonates more accurately and precisely identifies caries lesions and early stage caries lesions in teeth compared to conventional X-ray systems.

### Example 30

### Inhibition of Demineralization Progression Using Methylene Disphosphonate

Tooth specimens having caries lesions are contacted with methylene diphosphonate (MDP) to determine whether this bisphosphonate is effective to inhibit caries progression.

Teeth with caries were treated with MDP or water (control) and were then soaked for ten days at 25° C. in a demineralization solution (2.2 mM CaCl₂, 2.2 mM NaH₂PO₄, 0.05 M acetic acid, pH 4.4). The teeth were removed from the demineralization solution and the size of the caries after demineralization treatment was compared to the size of the caries prior to soaking in the demineralization solution. Data show significant progression of the caries lesion in control teeth treated with the water control prior to soaking in the demineralization solution (FIGS. 41C-D). Most importantly, caries lesions treated with MDP prior to soaking in demineralization showed a reduction in demineralization progression. (FIGS. 41A-B). The data show that MDP bisphosphonate inhibited enamel demineralization and caries progression in the teeth.

### Example 31

### Analysis of Inhibition of Caries Progression Using Bisphosphonates

To determine the ability of bisphosphonates to inhibit caries progression, ICG-bisphosphonate derivatives are administered to teeth prior to treatment with demineralization solutions.

Human extracted permanent premolars and molars without restorations and significant cavitated lesion are collected. Artificial caries lesions (n=50) of similar depth in the whole non-carious human teeth, are produced using methods similar to those previously described (Yamazaki H. et al. 2007 Arch. Oral Biol. 52(2):110-120; and Yamazaki H. et al. 2008 J Dent Res 87(6): 569-574). Surfaces of each tooth are coated with UV light-curing resin and nail varnish, except for a one millimeter window on interproximal surface. Caries forming solutions containing lactic acid, calcium, phosphate and sodium azide as a bactericide are prepared. Initial artificial lesions of predetermined depths (about 200 μm) are produced within the one millimeter window using the caries forming solutions. The amount of time need for the demineralization solution to generate lesions is varied and an optimum time is chosen (Margolis H. C. et al. 1999 J Dent Res. 78(7): 1326-1335). A High Resolution 3D X-ray Microscopy (Xradia) is used to confirm the presence and approximate depth of the caries lesion.

Following confirmation of artificial lesion preparation, sets of specimens from the extracted teeth (at least 40) are treated with NIR fluorescence bisphosphonate derivative, as described in FIG. 42. The remaining specimens serve as a control group and are treated with water. Specimens are placed in individual bottles containing a demineralizing solution (2.2 mM CaCl₂, 2.2 mM NaH₂PO₄, 0.05 M acetic acid, pH 4.4) at 25° C. Teeth are exposed to the demineralization solution for up to twelve weeks, as described (FIG. 42), and solutions are changed weekly. One set of teeth are treated with NIR fluorescent bisphosphonate derivative applied onto caries lesion (1.0 μl per lesion), prior to exposure to demineralization solution, and at four weeks and eight weeks as described in the FIG. 42. Teeth under process of demineralization are scanned by a High Resolution 3D X-ray Microscopy (Xradia) at four weeks, eight weeks and twelve weeks. The depth and shape of lesion are measured using a Xradia microscope.

At twelve weeks of demineralization, a thin longitudinal section (130-160 μm) that includes the lesion is prepared in each tooth. Mineral (e.g., calcium) percentage as a function of depth is determined for representative sections from each lesion using quantitative microradiography (Yamazaki H et al. 2007 Arch. Oral Biol. 52(2):110-120; and Yamazaki H. et al. 2008 J Dent Res 87(6):569-574). The mean mineral profile following the demineralization is divided by the mean mineral profile of initial lesion to quantify the amount of demineralization with respect to the initial lesion size (e.g., % mineral recovery). Initial lesion size is defined as the numerically integrated area of the mean mineral profile of the initial lesion of the control set (n=10).

It is expected that bisphosphonate compounds inhibit enamel caries lesion progression, and that bisphosphonate compounds are effective remineralization agents for detecting early caries lesions and incipient lesions with depths between 20-500 μm.

1. A method for enhancing remineralization of a tooth of a subject and inhibiting caries progression and loss of hydroxyapatite, the method comprising: contacting the tooth or oral cavity of the subject with a composition comprising at least one selected from bisphosphonate and a pyrophosphonate, wherein the composition binds to the tooth, an area of the tooth having a caries lesion or both; wherein the tooth of the subject comprises less porous hydroxyapatite, and the caries progression or the loss of the hydroxyapatite is inhibited, compared to a tooth not contacted.

2. The method according to claim 1, wherein the composition binds to hydroxyapatite.

3-5. (canceled)

6. The method according to claim 1, wherein the composition comprises at least one molecule selected from the group consisting of: a dye, a stain, a pigment, a polymer, a nanoparticle, and a peptide.

7. (canceled)

8. The method according to claim 6, wherein the composition comprises the molecule attached by a covalent bond or ionic bond.

9. The method according to claim 6, wherein the molecule comprises at least one selected from the group consisting of: a tetracycline, HiLyte Fluor, a Qdot, Cy7, CardioGreen (ICG), IR820 (ICG), Far-Green two, AngioSense 750, Genhance 750, AngioSpark, 750, Alexa Fluor 750, Indocyanine Green, Doxorubicin, Riboflavin, Chlorophyll A, a bacterial Chlorophyll, and a porphyrin.

10. The method according to claim 1, wherein the composition comprises at least one of: OsteoSense 750, zoledronate, aledronate, pamidronate, risedronate, ibandronate, incadronate, minodronate, olpadronate, neridronate, etidoronate, clodronate, tiludronate, and methylene bisphosphonate.

11. (canceled)

12. The method according to claim 1, wherein the composition further comprises at least one of: an antibacterial agent, an anti-microbial agent, and an anti-fungal agent.

13. The method according to claim 1, wherein the composition is formulated for delivery to the tooth or the oral cavity, for example the composition is formulated as at least one from the group of: a gel, a solution, an emulsion, an ointment, a paste, a cream, a gel, a powder, an aerosol, and a patch.

14. The method according to claim 1, further comprising after contacting the tooth or the oral cavity of the subject with the compound:
illuminating the tooth, the oral cavity or both; and,
detecting a wavelength of fluorescence emission, thereby monitoring the amount or extent of the inhibition of caries progression or the loss of hydroxyapatite of the tooth.

15. The method according to claim 1, wherein contacting is performed in vivo.

16. The method according to claim 1, wherein contacting is performed ex vivo.

17. The method according to claim 1, wherein the method further comprises prior to contacting the tooth or the oral cavity with the compound, visualizing a defect in the tooth.

18. (canceled)

19. The method according to claim 1, the method further comprises prior to contacting the tooth or the oral cavity with the composition at least one step selected from the group of: removing loose or diseased bone material, cleaning the tooth, comparing the bone material to a previous image of the tooth, detecting a caries in the tooth, and rinsing the tooth with a fluid.

20. (canceled)

21. The method according to claim 1, wherein contacting comprises adding the composition using an applicator, wherein the applicator is selected from the group of: a syringe, a brush, a swab, a sprayer, a sponge, a gauze, a bite wing plate, a dropper, a strip, a tape, a tray, and a string.

22. The method according to claim 1, further comprising after contacting the tooth or the oral cavity of the subject with the compound, contacting the tooth or oral cavity with a remineralization agent, for example the remineralization agent comprises at least one of calcium or phosphate.

23-30. (canceled)

31. A kit for enhancing remineralization of a tooth of a subject and inhibiting caries progression and loss of hydroxyapatite, the kit comprising:
a composition comprising at least one of a bisphosphonate and a pyrophosphonate;
instructions for use; and,
a container.

32-34. (canceled)

35. The kit according to claim 31, wherein the composition comprises at least one of: OsteoSense 750, aledronate, pamidronate, resedronate, ibandronate, etidoronate, clodronate, tiludronate, and methylene bisphosphonate.

36. The kit according to claim 31, further comprising a fluid for removing excess composition from the tooth.

37. The kit according to claim 31, wherein the composition is formulated as at least one selected from the group of a gel, a paste, a fluid, an emulsion, a patch, a capsule, an ointment, and a pill.

38. A product containing a composition for enhancing remineralization of a tooth of a subject and inhibiting caries progression and loss of hydroxyapatite, the composition comprising: at least one of a bisphosphonate and a pyrophosphonate, wherein the composition binds to hydroxyapatite in the tooth and an area of the tooth having a caries lesion.

39-45. (canceled)
